(19) United States
(12) Patent Application Publication
(10) Pub. No.: US 2023/0192692 A1
(43) Pub. Date: Jun. 22, 2023

(54) MACROCYCLIC AND CAGE-LIKE MOLECULE BASED ON BIPHEN[n]ARENE AND DERIVATIVE, SYNTHESIS METHOD AND USE THEREOF

(71) Applicant: TIANJIN NORMAL UNIVERSITY, Tianjin (CN)

(72) Inventors: Chunju LI, (US); Kaidi XU, (US); ZhiYuan ZHANG, (US)

(21) Appl. No.: 17/754,924

(22) PCT Filed: Oct. 10, 2020
(86) PCT No.: PCT/CN2020/120189

§ 371 Date: Apr. 15, 2022

(30) Foreign Application Priority Data
Oct. 15, 2019 (CN) 201910975631.8

(51) Int. Cl. C07D 471/08; C07C 43/205; C07D 513/18; C07C 49/755; C07D 487/18; C07C 50/34; C07D 487/22; C07C 49/255; C07F 5/02; C07C 59/68; C07C 51/41; C07C 37/50

(52) U.S. Cl. CPC C07D 471/08; C07C 43/2055; C07D 513/18; C07C 49/755; C07D 487/18; C07C 50/34; C07D 487/22; C07C 49/255; C07F 5/027; C07C 59/68; C07C 51/41; C07C 37/50

(57) ABSTRACT

A series of new macrocycles and cage-like molecules are obtained in a high yield from a bis-(2,4-dialkoxyphenyl)arene (naphthalene, anthracene, pyrene, porphyrin, etc.) or a tris-(2,4-dialkoxyphenyl)arene (benzene, sym-tribenzobenzene) and paraformaldehyde under the catalysis of a Lewis acid. In addition, perhydroxybiphenylarenes (tetrabiphenyl trimer, naphthalene dimer, etc.) can be obtained by means of demethylation, and a variety of water-soluble derivatives can be obtained by further modification, with same exhibiting a good bond ability for guest molecules (purpurine, etc.). Moreover, the functional group introduced into the backbone enables the macrocycle to have excellent adsorption and separation capabilities and a photophysical property. The macrocyclic and cage-like molecules have commercially available raw materials, are simple to synthesize, have a high yield, and are convenient to modify, such that same have wide application prospects in gas adsorption and separation, facilitate performance improvement of luminescent materials, perform adsorption of water-soluble toxic substances, etc.

The present application claims priority to Chinese Patent Application No. 201910975631.8 filed with China National Intellectual Property Administration on Oct. 15, 2019 and entitled “MACROCYCLIC AND CAGE-LIKE MOLECULES BASED ON BIPHENYLARENE AND DERIVATIVE COMPOUNDS, SYNTHESIS METHOD THEREFOR AND USE THEREOF”, the content of which is incorporated herein by reference in its entirety.

### TECHNICAL FIELD

The present disclosure relates to synthesis and derivatization of supramolecular macrocyclic and cage-like molecules, and particularly to a macrocyclic and cage-like molecule based on biphen[n]arene and derivative, synthesis method and use thereof.

### BACKGROUND

Since the discovery of the first-generation macrocyclic host crown ethers, macrocyclic molecules have proven essential for recognition and assembly, and thus they have wide application in the fields of chemistry, materials and biology. The wide application makes the synthesis of new macrocycles extremely appealing. Chemists have put a great deal of effort into the synthesis of macrocyclic compounds such as Texas rings, ExBox, pillararenes, cucurbituril, pillararenes, calixarenes, caliximidazoles, calixpyrroles, and cyanostars. However, their modification is mainly limited to the edge or side chain moieties, since any change in the framework may affect the ring formation reaction. Besides, for most macrocyclic compounds, the complicated synthesis and low yields have been a great hindrance to their further development and application. Thus, macrocyclic compounds with both multiple modifiable sites and high yields are still rare. However, the extended biphenyl[n]arenes synthesized by us have both of these advantages. Moreover, earlier work has proved that our biphenyl arenes have great potential to be applied in the fields of materials, biology, environment, etc.

### SUMMARY

Aiming to solve the problems of complicated synthetic routes, low yields and limited modification sites, etc., of macrocycles, the present disclosure provides a simple, efficient and universal synthesis method for a macrocyclic and cage-like molecule based on biphen[n]arene, and develops a method for further derivatization. The method uses reactants such as bis-(2,4-dialkoxyphenyl) monomer and formaldehyde to synthesize supramolecular macrocyclic and cage-like products through a one-pot process with high yields, and is able to achieve further derivatization to obtain a series of water-soluble or liposoluble derivatives, significantly improving the application potential of the supramolecular macrocyclic and cage-like molecule based on biphen[n]arene.

The present disclosure provides the following technical solutions.

The present disclosure first provides the following three classes of compounds:

(1) a monomer of supramolecular macrocyclic and cage-like molecule based on biphen[n]arene;

(2) a supramolecular macrocyclic and cage-like molecule compound based on biphen[n]arene; and

(3) a derivative compound of supramolecular macrocyclic and cage-like molecule based on biphen[n]arene.

According to an embodiment of the present disclosure, the monomer of supramolecular macrocycle based on biphen[n]arene is selected from: a macrocyclic monomer with methoxy side chain, a monomer of cage-like molecule, and a macrocyclic monomer with a dibutoxyl or 4-methoxy-24(5-bromo-n-pentyloxy) side chain.

According to an embodiment of the present disclosure, the macrocyclic monomer with methoxy side chain is selected from the compound of formula I.

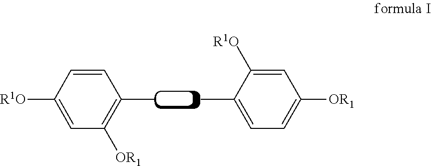

In formula I, R¹ is selected from methyl;

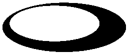

is selected from the structures listed in the following table,

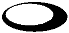

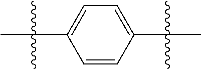 [SMILES: *c1ccc(*)cc1]

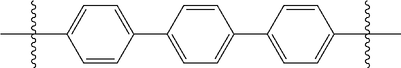 [SMILES: *c1ccc(-c2ccc(-c3ccc(*)cc3)cc2)cc1]

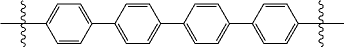 [SMILES: *c1ccc(-c2ccc(-c3ccc(-c4ccc(*)cc4)cc3)cc2)cc1]

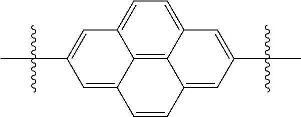 [SMILES: *c1cc2ccc3cc(*)cc4ccc(c1)c2c34]

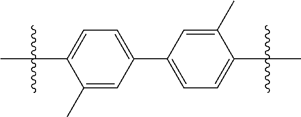 [SMILES: *c1ccc(-c2ccc(*)c(C)c2)cc1C]

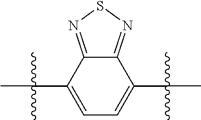 [SMILES: *c1ccc(*)c2nsnc12]

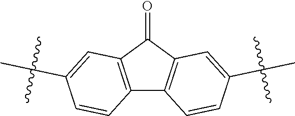 [SMILES: *c1ccc2c(c1)C(=O)c1cc(*)ccc1-2]

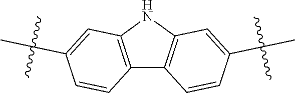 [SMILES: *c1ccc2c(c1)[nH]c1cc(*)ccc12]

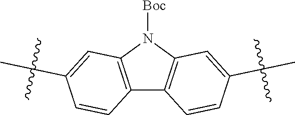 [SMILES: *c1ccc2c3ccc(*)cc3n(C(=O)OC(C)(C)C)c2c1]

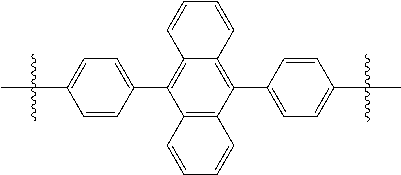 [SMILES: *c1ccc(-c2c3ccccc3c(-c3ccc(*)cc3)c3ccccc23)cc1]

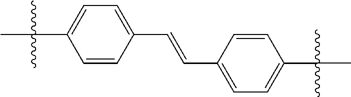 [SMILES: *c1ccc(/C=C/c2ccc(*)cc2)cc1]

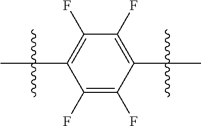 [SMILES: *c1c(F)c(F)c(*)c(F)c1F]

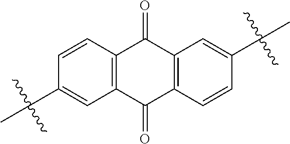 [SMILES: *c1ccc2c(c1)C(=O)c1ccc(*)cc1C2=O]

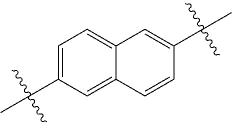 [SMILES: *c1ccc2cc(*)ccc2c1]

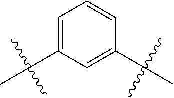 [SMILES: *c1cccc(*)c1]

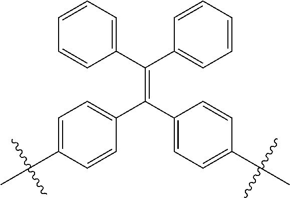 [SMILES: *c1ccc(C(=C(c2ccccc2)c2ccccc2)c2ccc(*)cc2)cc1]

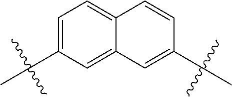 [SMILES: *c1ccc2ccc(*)cc2c1]

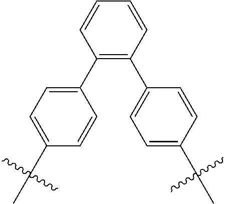 [SMILES: *c1ccc(-c2ccccc2-c2ccc(*)cc2)cc1]

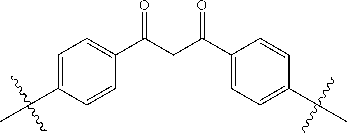 [SMILES: *c1ccc(C(=O)CC(=O)c2ccc(*)cc2)cc1]

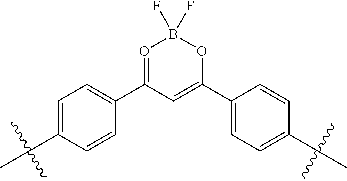

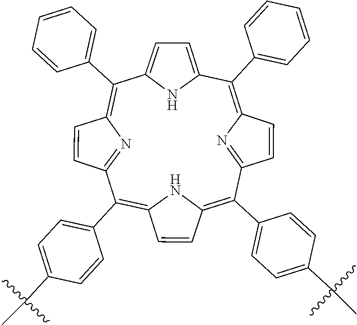 [SMILES: *c1ccc(-c2c3nc(c(-c4ccccc4)c4ccc([nH]4)c(-c4ccccc4)c4nc(c(-c5ccc(*)cc5)c5ccc2[nH]5)C=C4)C=C3)cc1]

According to an embodiment of the present disclosure, the monomer of cage-like molecule is selected from the compound of formula II,

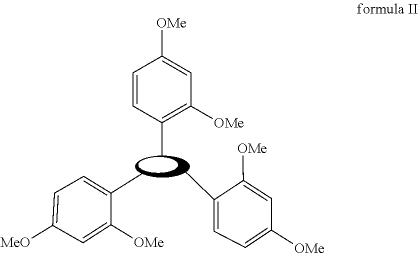

In formula II,

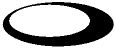

is selected from

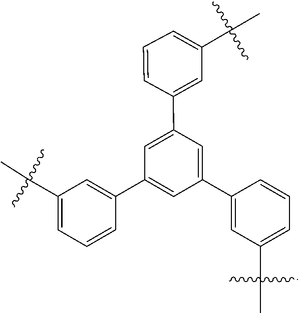 [SMILES: *c1cccc(-c2cc(-c3cccc(*)c3)cc(-c3cccc(*)c3)c2)c1]

According to an embodiment of the present disclosure, the a macrocyclic monomer with a dibutoxyl or 4-methoxy-2-(5-bromo-n-pentyloxy) side chain is selected from the compound of formula III,

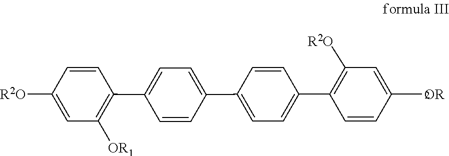

wherein R¹ and R² are selected from n-butyl; or R¹ is selected from 5-bromo-n-pentyl, and R² is selected from methyl.

According to an embodiment of the present disclosure, the supramolecular macrocyclic and cage-like molecule compound based on biphen[n]arene is selected from: a macrocyclic compound with trimer or higher degree of polymerization synthesized from a linear molecule, a dimeric supramolecular macrocyclic compound prepared from a V-shaped molecule, a supracage-like molecule compound constructed from monomer molecules having three 2,4-dialkoxyphenyl groups, and supramolecular macrocyclic compounds having different repeat units.

According to an embodiment of the present disclosure, the macrocyclic compound with trimer or higher degree of polymerization synthesized from a linear molecule is selected from the compound of formula IV or the compound of formula V.

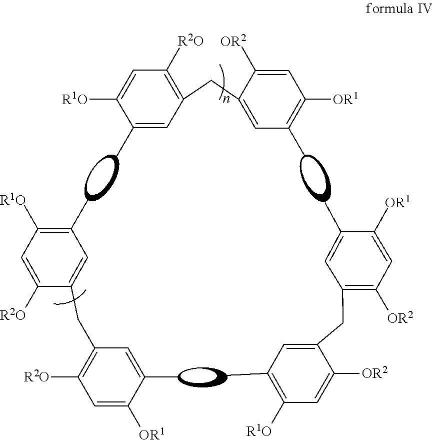

wherein

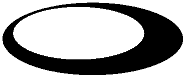

and R¹ and R² are defined as below,

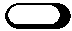

is:

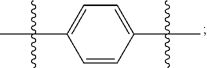 [SMILES: *c1ccc(*)cc1]

R¹=R²=Me; n=3 or 5

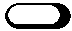

is:

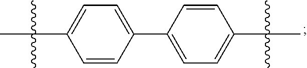 [SMILES: *c1ccc(-c2ccc(*)cc2)cc1]

R¹=R²=Me; n=3

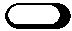

is:

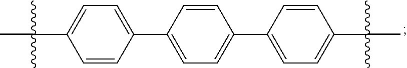 [SMILES: *c1ccc(-c2ccc(-c3ccc(*)cc3)cc2)cc1]

R¹=R²=Me; n=3-6

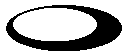

is:

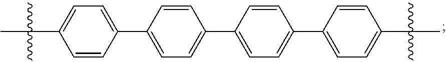 [SMILES: *c1ccc(-c2ccc(-c3ccc(-c4ccc(*)cc4)cc3)cc2)cc1]

R¹=R²=Me; n=3

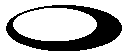

is:

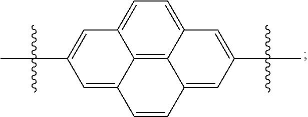 [SMILES: *c1cc2ccc3cc(*)cc4ccc(c1)c2c34]

R¹=R²=Me; n=3-4

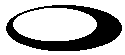

is:

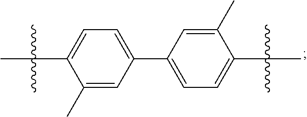 [SMILES: *c1ccc(-c2ccc(*)c(C)c2)cc1C]

R¹=R²=Me; n=3-6

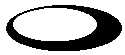

is:

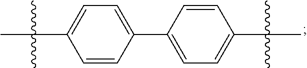 [SMILES: *c1ccc(-c2ccc(*)cc2)cc1]

R¹=R²=n-Butyl; n=3

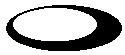

is:

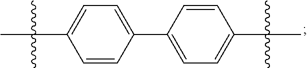 [SMILES: *c1ccc(-c2ccc(*)cc2)cc1]

R¹=5-bromo-n-pentyl; R²=Me; n=3-4

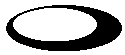

is:

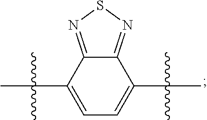 [SMILES: *c1ccc(*)c2nsnc12]

R¹=R²=Me; n=3

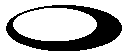

is:

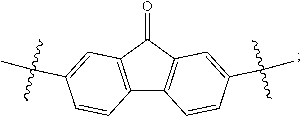 [SMILES: *c1ccc2c(c1)C(=O)c1cc(*)ccc1-2]

R¹=R²=Me; n=2-5

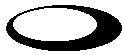

is:

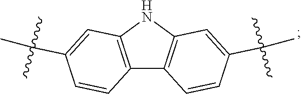 [SMILES: *c1ccc2c(c1)[nH]c1cc(*)ccc12]

R¹=R²=Me; n=3

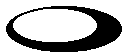

is:

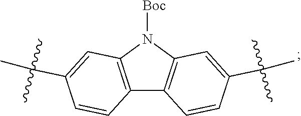 [SMILES: *c1ccc2c3ccc(*)cc3n(C(=O)OC(C)(C)C)c2c1]

R¹=R²=Me; n=3

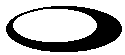

is:

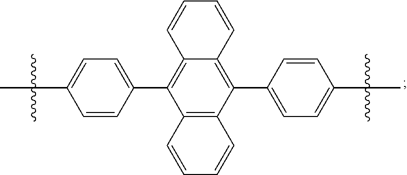 [SMILES: *c1ccc(-c2c3ccccc3c(-c3ccc(*)cc3)c3ccccc23)cc1]

R¹=R²=Me; n=3

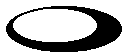

is:

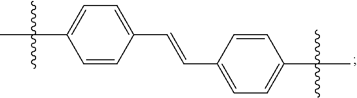 [SMILES: *c1ccc(/C=C/c2ccc(*)cc2)cc1]

R¹=R²=Me; n=3

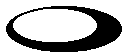

is:

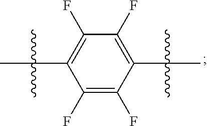 [SMILES: *c1c(F)c(F)c(*)c(F)c1F]

R¹=R²=Me; n=3

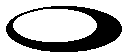

is:

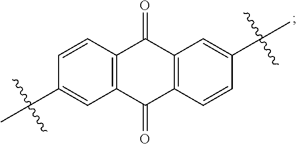 [SMILES: *c1ccc2c(c1)C(=O)c1ccc(*)cc1C2=O]

R¹=R²=Me; n=3

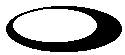

is:

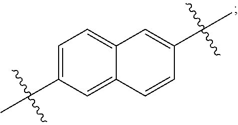 [SMILES: *c1ccc2cc(*)ccc2c1]

R¹=R²=Me; n=3

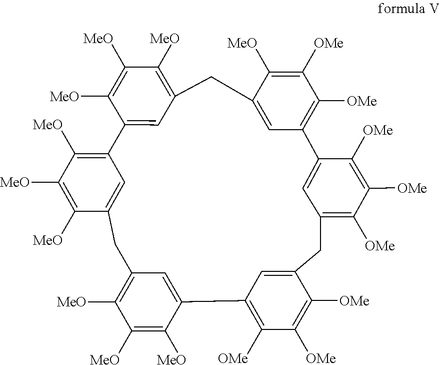 [SMILES: COc1c2cc(c(OC)c1OC)-c1cc(c(OC)c(OC)c1OC)Cc1cc(c(OC)c(OC)c1OC)-c1cc(c(OC)c(OC)c1OC)Cc1cc(c(OC)c(OC)c1OC)-c1cc(c(OC)c(OC)c1OC)C2]

According to an embodiment of the present disclosure, the dimeric supramolecular macrocycle prepared from a V-shaped molecule is selected from the compound of formula VI,

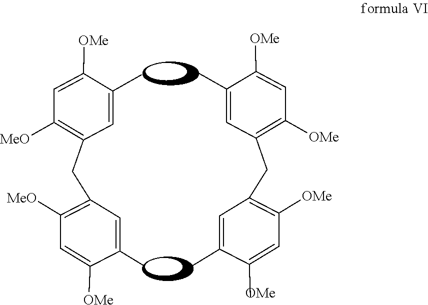

In formula VI,

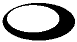

is selected from:

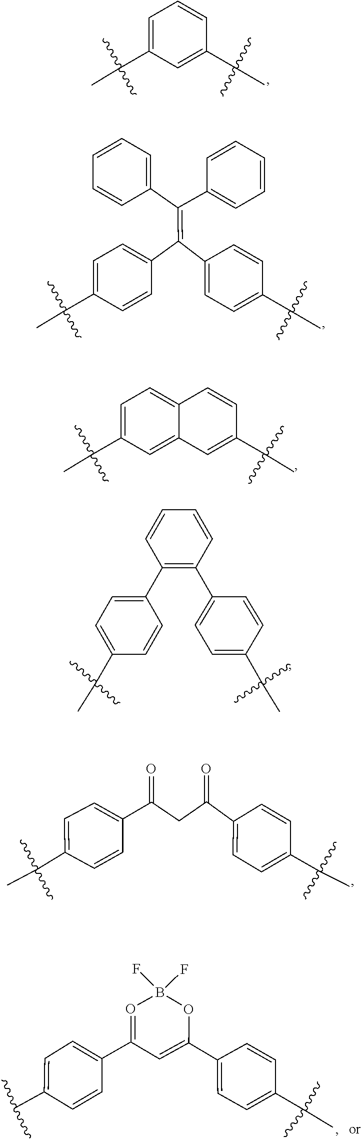

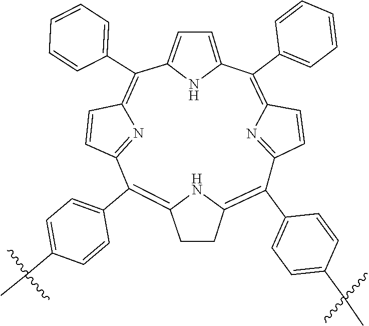 [SMILES: *c1ccc(-c2c3nc(c(-c4ccccc4)c4ccc([nH]4)c(-c4ccccc4)c4nc(c(-c5ccc(*)cc5)c5[nH]c2CC5)C=C4)C=C3)cc1]

According to an embodiment of the present disclosure, the supracage-like molecule compound constructed from monomer molecules having three 2,4-dialkoxyphenyl groups is selected from the compound of formula VII.

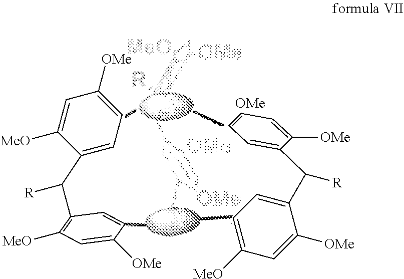

In the compound of formula VII:

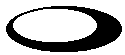

is:

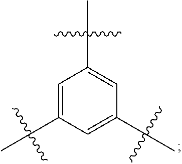 [SMILES: *c1cc(*)cc(*)c1]

R=H

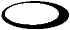

is:

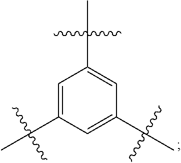 [SMILES: *c1cc(*)cc(*)c1]

R=

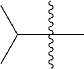 [SMILES: *C(C)C]

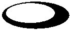

is:

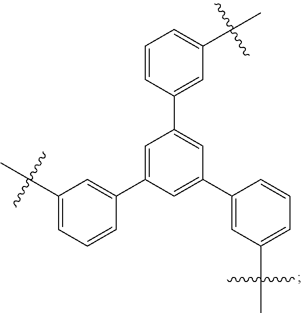 [SMILES: *c1cccc(-c2cc(-c3cccc(*)c3)cc(-c3cccc(*)c3)c2)c1]

R=H

According to an embodiment of the present disclosure, the supramolecular macrocyclic compound having different repeat units is selected from the compound of formula VIII,

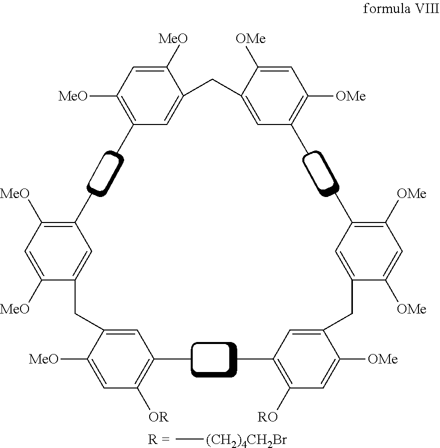

wherein

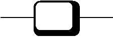

is selected from

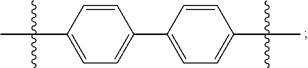 [SMILES: *c1ccc(-c2ccc(*)cc2)cc1 ; ";"]

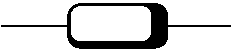

is selected from

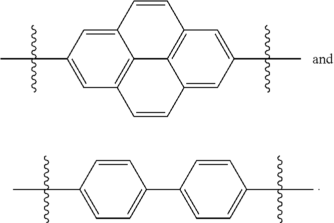

According to an embodiment of the present disclosure, the derivative compound of supramolecular macrocyclic and cage-like molecule based on biphen[n]arene is selected from: trimeric macrocyclic hydroxyl compounds formed from linear monomers, V-shaped dimeric macrocyclic hydroxyl compounds, cage-like molecule hydroxyl derivative compounds, water-soluble ammonium carboxylate derivative macrocyclic compounds, water-soluble sodium carboxylate derivative cage-like molecule compounds, water-soluble sulfonate salt derivative macrocyclic compounds, carbazole derivative macrocycles, and pyridine derivative macrocycles.

According to an embodiment of the present disclosure, the trimeric macrocyclic hydroxyl compound formed from linear monomers is selected from the following compound,

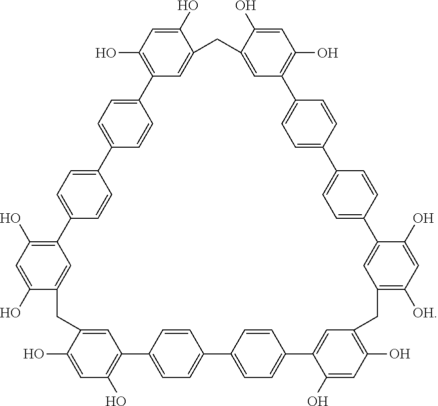 [SMILES: Oc1cc(O)c2cc1Cc1cc(c(O)cc1O)-c1ccc(cc1)-c1ccc(cc1)-c1cc(c(O)cc1O)Cc1cc(c(O)cc1O)-c1ccc(cc1)-c1ccc(cc1)-c1cc(c(O)cc1O)Cc1cc(c(O)cc1O)-c1ccc(cc1)-c1ccc-2cc1]

According to an embodiment of the present disclosure, the V-shaped dimeric macrocyclic hydroxyl compound is selected from the compound of formula V-1,

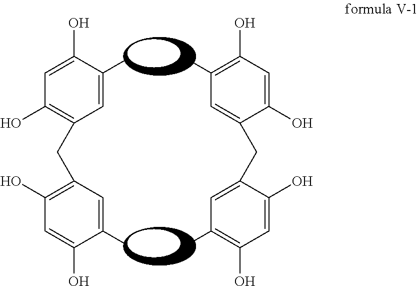 [SMILES: Cc1cc(Cc2cc(C)c(O)cc2O)c(O)cc1O ; Cc1cc(Cc2cc(C)c(O)cc2O)c(O)cc1O]

wherein

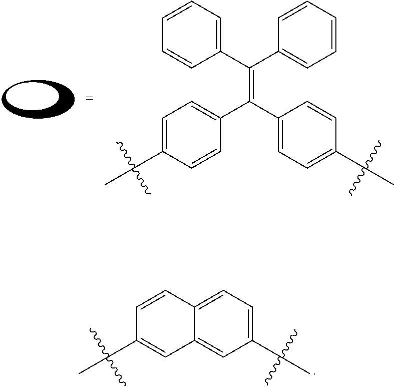 [SMILES: *c1ccc(C(=C(c2ccccc2)c2ccccc2)c2ccc(*)cc2)cc1 ; *c1ccc2ccc(*)cc2c1 ; "."]

According to an embodiment of the present disclosure, the cage-like molecule hydroxyl derivative compound is selected from the following compound,

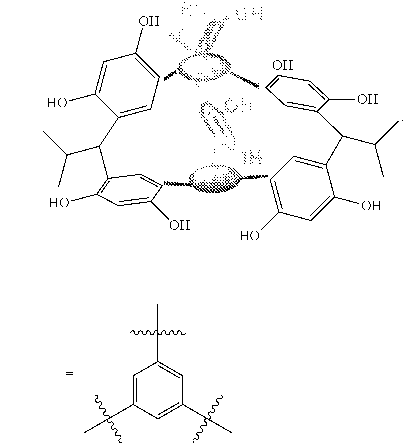

According to an embodiment of the present disclosure, the water-soluble ammonium carboxylate derivative macrocyclic compound is selected from the following compound:

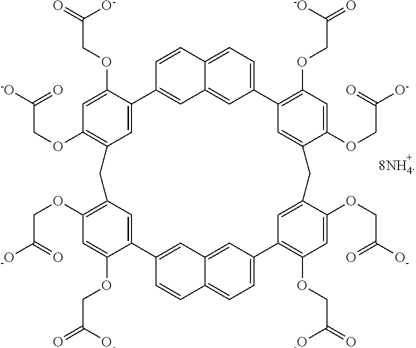

According to an embodiment of the present disclosure, the water-soluble sodium carboxylate derivative cage-like molecule compound is selected from the following compound:

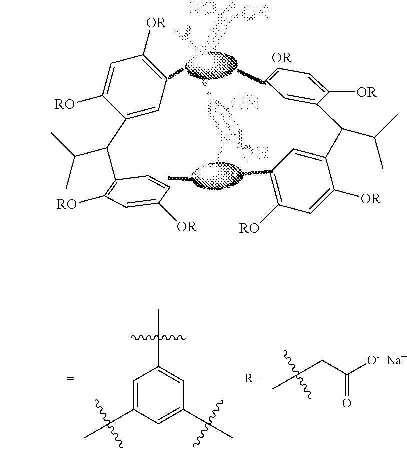

According to an embodiment of the present disclosure, the water-soluble sulfonate salt derivative macrocyclic compound is selected from the following compound:

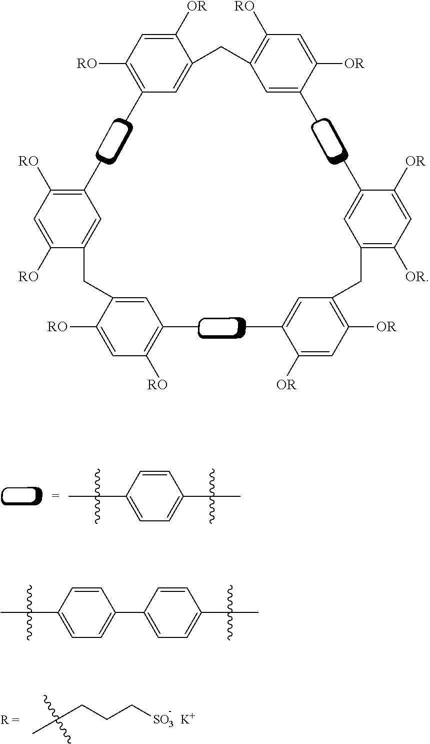

According to an embodiment of the present disclosure, the carbazole derivative macrocycle is selected from the following compound:

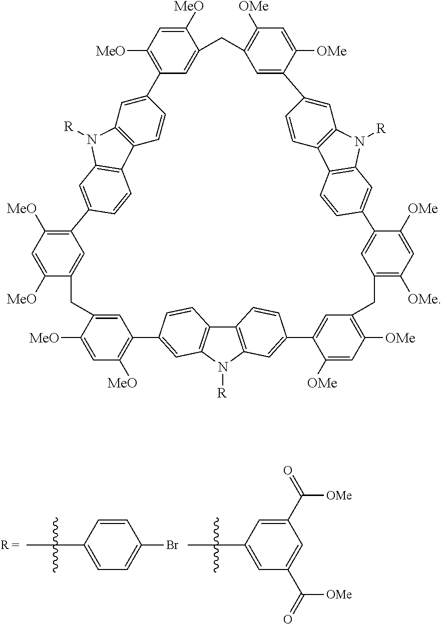

According to an embodiment of the present disclosure, the pyridine derivative macrocycle is selected from the following compound:

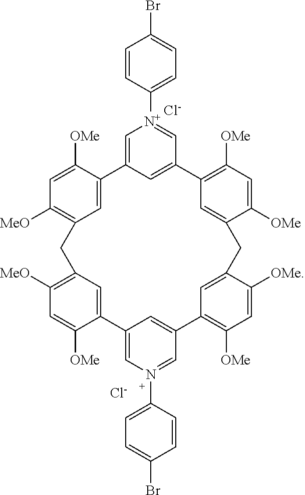

The present disclosure further provides a synthesis method for the monomer of supramolecular macrocyclic and cage-like molecule based on biphen[n]arene, the supramolecular macrocyclic and cage-like molecule compound based on biphen[n]arene and the derivative compounds thereof, wherein the synthesis method comprising the following steps: obtaining a bis-(2,4-dialkoxyphenyl)arene and tris-(2,4-dialkoxyphenyl)arene by Suzuki coupling reaction, then dissolving the bis-(2,4-dialkoxyphenyl)arene or tris-(2,4-dialkoxyphenyl)arene in a halohydrocarbon solvent, adding an aldehyde reactant, obtaining a series of supramolecular macrocyclic and cage-like molecule based on biphen[n]arene hosts through cyclization under catalysis by a Lewis acid, and obtaining derivative compounds of the macrocycles and cage-like compounds by further derivatization.

According to an embodiment of the present disclosure, the halohydrocarbon solvent is at least one selected from: dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, and monobromodecane.

According to an embodiment of the present disclosure, the aldehyde reactant is selected from paraformaldehyde and isobutyraldehyde.

According to an embodiment of the present disclosure, the synthesis method comprises the following steps:

(1) synthesis of the monomer of supramolecular macrocyclic and cage-like molecule based on biphen[n]arene;

(2) synthesis of the supramolecular macrocyclic and cage-like molecule compound based on biphen[n]arene; and

(3) synthesis of the derivative compound of supramolecular macrocyclic and cage-like molecule based on biphen[n]arene:

wherein a synthesis method for the monomer of supramolecular macrocyclic and cage-like molecule based on biphen[n]arene of step (1) is as follows:

[1] Preparation of a Monomer of Supramolecular Macrocycle Based on biphen[n]arene

dissolving a dibromide or an iodide and 2,4-dimethoxyphenylboronic acid in an aqueous solution of dioxane (dioxane:water=5:1), then adding tetrakis(triphenylphosphine)palladium catalyst and sodium carbonate, and stirring the mixture at reflux overnight; ater the reaction is completed, cooling the reaction mixture to room temperature, removing the solvent by rotary evaporation, dissolving the residue in dichloromethane, and washing the resulting solution with water three times; drying the organic layer over anhydrous Na₂SO₄ and removing the solvent again by rotary evaporation, and preparing the residue for column chromatography to isolate the monomer:

[2] Preparation of a Monomer of the Cage-Like Molecule

dissolving a tribromide and 2,4-dimethoxyphenylboronic acid in an aqueous solution of dioxane (dioxane:water=5:1), then adding tetrakis(triphenylphosphine)palladium catalyst and sodium carbonate, and stirring the mixture at reflux overnight; after the reaction is completed, cooling the reaction mixture to room temperature, removing the solvent by rotary evaporation, dissolving the residue in dichloromethane, and washing the resulting solution with water three times; drying the organic layer over anhydrous Na₂SO₄ and removing the solvent again by rotary evaporation, and preparing the residue for column chromatography to isolate the monomer:

[3] Preparation of a Macrocyclic Monomer with Dibutoxyl or 4-methoxy-2-(5-bromo-n-pentyloxy) Side Chain

1) Synthesis of a Macrocyclic Monomer with Dibutoxy Side Chain

adding excessively n-butyl bromide to a three-necked flask and heating n-butyl bromide at reflux, dissolving 4-bromo-resorcinol in acetonitrile and adding dropwise the resulting solution to the reaction system, and allowing the reaction system to react overnight; after the reaction is completed, stopping the heating, and filtering the mixture to remove potassium carbonate; concentrating the reaction solution by rotary evaporation, and subjecting the residue to column chromatography to isolate 4-bromo-1,3-dibutoxybenzene reaction product; subsequently, dissolving completely 4-bromo-1,3-dibutoxybenzene in an aqueous solution of 1,4-dioxane (dioxane:water=5:1), then adding 4,4′-biphenyldiboronic acid, tetrakis(triphenylphosphine)palladium and sodium carbonate, and heating the mixed system to 100° C. and refluxing overnight; after the reaction is completed, cooling the reaction mixture to room temperature, removing the solvent by rotary evaporation, dissolving the residue in dichloromethane, and washing the resulting solution with water three times; drying the organic layer over anhydrous Na₂SO₄ and removing the solvent again by rotary evaporation, and preparing the residue for column chromatography to isolate the monomer:

2) Synthesis of a Macrocyclic Monomer with 4-methoxy-2-(5-bromo-n-pentyloxy) Side Chain

adding excessively 1,5-dibromopentane to a three-necked flask and heating 1,5-dibromopentane at reflux, starting dissolving 2-bromo-5-methoxyphenol in acetonitrile and adding dropwise the resulting solution to the reaction system, and allowing the reaction system to react overnight; after the reaction is completed, stopping the heating, and filtering the mixture to remove potassium carbonate; concentrating the reaction solution by rotary evaporation, and subjecting the residue to column chromatography to isolate 4-methoxy-2-(5-bromo-n-pentyloxy)bromobenzene reaction product; subsequently, dissolving completely 4-methoxy-2-(5-bromo-n-pentyloxy)bromobenzene in an aqueous solution of 1,4-dioxane (dioxane:water=5:1), then adding 4,4′-biphenyldiboronic acid, tetrakis(triphenylphosphine)palladium and sodium carbonate, and heating the mixed system to 100° C. and refluxing overnight; after the reaction is completed, cooling the reaction mixture to room temperature, removing the solvent by rotary evaporation, dissolving the residue in dichloromethane, and washing the resulting solution with water three times; drying the organic layer over anhydrous Na₂SO₄ and removing the solvent again by rotary evaporation, and preparing the residue for column chromatography to isolate the monomer;

wherein a synthesis method for the supramolecular macrocyclic and cage-like molecule compound based on biphen[n]arene of step (2) is as follows:

[1] Synthesis of a Supramolecular Macrocycle with Trimer or Higher Degree of Polymerization from a Molecule Having a Linear Structure:

using a bis-(2,4-dialkoxyphenyl)arene having a linear structure and an aldehyde reactant as starting materials and a haloalkane as a solvent, adding a Lewis acid catalyst after the starting materials are dissolved, monitoring the reaction by thin-layer chromatography (TLC), quenching the reaction with saturated aqueous sodium bicarbonate solution after the reaction is completed, washing the mixture with saturated aqueous sodium chloride solution, drying the mixture over anhydrous sodium sulfate, and subjecting the resulting mixture to silica gel column chromatography to isolate a ring formation product with trimer or higher degree of polymerization; wherein the halohydrocarbon solvent is at least one selected from: dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, and monobromodecane: the aldehyde reactant is selected from paraformaldehyde and isobutyraldehyde;

[2] Preparation of a Dimeric Macrocyclic Arene from a V-Shaped Monomer:

using a bis-(2,4-dialkoxyphenyl)arene having a V-shaped structure and an aldehyde reactant as starting materials and a haloalkane as a solvent, adding a Lewis acid catalyst after the starting materials are dissolved, monitoring the reaction by thin-layer chromatography (TLC), quenching the reaction with saturated aqueous sodium bicarbonate solution after the reaction is completed, washing the mixture with saturated aqueous sodium chloride solution, drying the mixture over anhydrous sodium sulfate, and subjecting the resulting mixture to silica gel column chromatography to isolate a dimeric ring formation product; wherein the halohydrocarbon solvent is at least one selected from: dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, and monobromodecane; the aldehyde reactant is selected from paraformaldehyde and isobutyraldehyde;

[3] Synthesis of a Cage-Like Molecule Compound from tris-(2,4-dialkoxyphenyl)arene:

using a tris-(2,4-dialkoxyphenyl)arene and paraformaldehyde or isobutyraldehyde as starting materials (the molar ratio of tris-(2,4-dialkoxyphenyl)arene to paraformaldehyde or isobutyraldehyde is about 1:5) and a haloalkane as a solvent, adding a Lewis acid catalyst after the starting materials are dissolved, monitoring the reaction by thin-layer chromatography (TLC), quenching the reaction with saturated aqueous sodium bicarbonate solution after the reaction is completed, washing the mixture with saturated aqueous sodium chloride solution, drying the mixture over anhydrous sodium sulfate, and subjecting the resulting mixture to silica gel column chromatography to isolate a cage-like molecule compound product; wherein the halohydrocarbon solvent is at least one selected from: dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, and monobromodecane; the aldehyde reactant is selected from paraformaldehyde and isobutyraldehyde;

[4] Preparation of a Supramolecular Macrocyclic Compound in which a Macrocycle has Different Units by Regulating Proportions of Different Monomer Molecules to Achieve Copolymerization of the Different Monomers:

adding two bis-(2,4-dialkoxyphenyl)arenes to a reaction flask in a molar ratio of 1:5, then adding a paraformaldehyde in an amount that is twice the total amount of substance of the two derivatives, adding a Lewis acid catalyst after dissolution in a haloalkane, monitoring the reaction by thin-layer chromatography (TLC), quenching the reaction with saturated aqueous sodium bicarbonate solution after the reaction is completed, washing the mixture with saturated aqueous sodium chloride solution, drying the mixture over anhydrous sodium sulfate, and subjecting the resulting mixture to silica gel column chromatography to isolate a copolymeric three-membered macrocyclic compound; the halohydrocarbon solvent is at least one selected from: dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, and monobromodecane;

a synthesis method for the derivatization of macrocyclic and cage-like molecule based on biphen[n]arenes of step (3) is as follows:

[1] Synthesis of a Hydroxyl Macrocyclic Compound:

dissolving a biphenyl arene macrocycle in dichloromethane, adding 20 equivalents of boron tribromide compound to the reaction system, after 1 day of reaction, adding dropwise the reaction mixture to a mixture of ice and water to precipitate a light purple powder, and performing suction filtration to obtain a hydroxyl derivative macrocycle product:

[2] Synthesis of a Carboxylic Acid Water-Soluble Macrocycle and a Carboxylic Acid Water-Soluble Cage-Like Molecule:

dissolving a hydroxyl macrocyclic compound product in acetonitrile or acetone, then adding K₂CO₃, refluxing the mixture for 2 h, adding ethyl bromoacetate, refluxing the mixture for another 48 h, cooling the reaction mixture to room temperature after the reaction is completed, filtering the reaction mixture, washing with dichloromethane multiple times, removing the solvent by vacuum rotary evaporation, adding a small amount of dichloromethane so that the solid is just dissolved, then adding a large amount of petroleum ether so that a large amount of solid is subsequently precipitated, and performing suction filtration under reduced pressure to obtain the desired product; dissolving the product in a mixed solution of 50 mL of tetrahydrofuran (THF) and 20 mL of an aqueous solution of sodium hydroxide (a mass concentration of 20%), stirring the resulting solution at reflux for 10 h, removing THF by rotary evaporation, adding 20 mL of water, adding hydrochloric acid for acidification until a pH test paper shows weak acidity, performing suction filtration under reduced pressure to obtain the desired product, and then adding gradually the carboxylic acid derivative macrocyclic and cage-like molecule to an alkali solution to obtain a carboxylate salt water-soluble macrocyclic and cage-like molecule compound of the corresponding alkali;

[3] Synthesis of a Sulfonated Water-Soluble Macrocycle

dissolving a hydroxyl macrocyclic compound in acetone, then adding K₂CO₃, stirring the mixture at reflux for 2 h, then adding 1 equivalent of propane sultone, stirring the mixture at reflux for another 3 days, cooling the mixture to room temperature after the reaction is completed, performing suction filtration, washing the filter cake twice with acetone, dissolving the resulting filter cake in water, purifying the resulting solution to remove potassium carbonate by about one week of dialysis with a dialysis bag; adding 800 mL of distilled water to a 1 L large beaker, then placing the dialysis bag in the water, fixing slightly the dialysis bag with a rubber band, adding a stirrer, stirring continuously the water, replacing the water in the beaker once every 2 hours, reducing the water replacing frequency after one day to once every half a day, and replacing the water once on the third day; and finally concentrating the aqueous solution in the dialysis bag by rotary evaporation to obtain a sulfonated water-soluble macrocycle product;

[4] Special Derivatization of Certain Macrocyclic Compounds

[1] Synthesis of a Carbazole Derivative Macrocycle:

\textcircled{1} Monomer Modification, and then Ring Closing:

dissolving bis-(2,4-dialkoxyphenyl)carbazole and 2 equivalents of p-dibromobenzene or methyl 5-bromoisophthalate in N,N-dimethylacetamide, adding 1 equivalent of copper(I) iodide and 6 equivalents of potassium carbonate, heating the mixture to 180° C. under nitrogen (argon) atmosphere for 24 h of reaction, cooling the mixture to room temperature after the reaction is completed, pouring the product into a saturated aqueous solution of NaCl, performing extraction with dichloromethane three times, performing drying over anhydrous sodium sulfate, and performing purification by column chromatography to obtain a product in the form of a white solid; dissolving the monomer above and 3 equivalents of paraformaldehyde in dichloromethane, adding boron trifluoride diethyl ether catalyst, and monitoring the reaction by thin-layer chromatography (TLC); after the reaction is completed, quenching the reaction with saturated aqueous sodium bicarbonate solution, washing the mixture with saturated aqueous sodium chloride solution, drying the mixture over anhydrous sodium sulfate, and subjecting the resulting mixture to silica gel chromatography to isolate a carbazole three-membered ring macrocycle product modified by p-dibromobenzene or methyl 5-bromoisophthalate;

\textcircled{2} Ring Closing, and then Modification:

dissolving bis-(2,4-dialkoxyphenyl)carbazole and 3 equivalents of paraformaldehyde in dichloromethane, adding 2 equivalents of boron trifluoride diethyl ether catalyst, monitoring the reaction by thin-layer chromatography (TLC), quenching the reaction with saturated aqueous sodium bicarbonate solution after the reaction is completed, washing the mixture with saturated aqueous sodium chloride solution, drying the mixture over anhydrous sodium sulfate, and subjecting the resulting mixture to silica gel chromatography to isolate a carbazole-modified three-membered ring product; dissolving a mixture of the carbazole-modified three-membered ring, 2 equivalents of p-dibromobenzene or methyl 5-bromoisophthalate, 1 equivalent of copper(I) iodide and 6 equivalents of potassium carbonate in N,N-dimethylacetamide, heating the resulting solution to 180° C. under nitrogen (argon) atmosphere for 24 h of reaction, then cooling the reaction solution to room temperature, pouring the product into a saturated aqueous solution of NaCl, performing extraction with dichloromethane three times, performing drying over anhydrous sodium sulfate, and performing purification by column chromatography to obtain a three-membered ring product modified by p-dibromobenzene or methyl 5-bromoisophthalate;

[2] Synthesis of a Pyridine Derivative Macrocycle:

\textcircled{1} Monomer Modification and then Ring Closing:

in a 100 mL three-necked flask, adding 2.4 g of 3,5-dibromopyridine and 3.6 g of 2,4-dimethoxyphenylboronic acid to a beaker in a molar ratio of 1:2, then adding 2.1 g of anhydrous sodium carbonate and 0.4 g of tetrakis(triphenylphosphine)palladium, using 80 mL of 1,4-dioxane and 20 mL of water as solvents, heating the resulting mixture at reflux in a 110° C. oil bath overnight, after the reaction is completed, evaporating sequentially the solvents, performing extraction with water and dichloromethane, and performing drying over anhydrous sodium sulfate, and separating out the organic phase; concentrating the obtained organic phase, and subjecting the residue to column chromatography for purification to isolate 3,5-bis(2,4-dimethoxyphenyl)pyridine in the form of a white solid; adding sequentially 1.7 g of 3,5-bis-(2,4-dimethoxyphenyl)pyridine monomer and 1.5 g of 2,4-dinitrochlorobenzene to a 50 mL round-bottomed flask, adding 5 mL of acetone, mixing well the mixture by ultrasonication, then heating the reactants at reflux overnight, after the reaction is completed, removing the solvent by rotary evaporation, adding a large amount of ethyl acetate, performing suction filtration, adding the filter cake to an acetonitrile solution, performing suction filtration, collecting the filtrate, concentrating the filtrate by rotary evaporation, adding a small amount of methanol to completely dissolve the residue, adding ethyl acetate, stirring the mixture for 1-4 h, and performing suction filtration to obtain an intermediate product; adding 0.5 g of the intermediate product to a 50 mL round-bottomed flask, adding 1 mL of ethanol, then adding 3 mL of water, mixing well the mixture, then adding 3.5 g of p-bromophenylamine, heating the mixture at reflux under nitrogen atmosphere for 1-2 days, cooling the mixture to room temperature, then adding ethyl acetate, and performing suction filtration; adding ethanol to the filtrate, removing the solvent by rotary evaporation, then adding a small amount of acetone to dissolve the solid, then adding a large amount of ethyl acetate, and performing suction filtration to obtain a modified pyridine arene derivative monomer; then weighing 1 g of the derivative arene monomer and 0.3 g of parafomaldehyde, pouring 150 mL of dichloromethane solvent for dissolution, adding 1.5 equivalents of boron trifluoride diethyl ether catalyst while stirring, monitoring the reaction by thin-layer chromatography, after 30 min of reaction, adding 100 mL of a saturated solution of sodium bicarbonate to quench the reaction, washing the mixture with 50 mL of a saturated solution of sodium chloride, drying the mixture over anhydrous sodium sulfate, and subjecting the resulting mixture to silica gel chromatography to obtain the target product:

\textcircled{2} Ring Closing, and then Modification:

dissolving 3,5-bis(2,4-dimethoxyphenyl)pyridine compound (3.52 g, 10 mmol) in 100 mL of dichloromethane, and adding paraformaldehyde (0.45 g, 15 mmol); stirring the mixture for a few minutes until complete dissolution is achieved, adding boron trifluoride diethyl ether catalyst to the mixture so that the reaction mixture gradually turns from colorless to light purple, stirring the mixture at room temperature for 25 min, monitoring the reaction by TLC, and quenching the reaction with a saturated solution of sodium bicarbonate when the starting materials are substantially consumed; separating out the organic phase, washing the organic phase once with water, and finally extracting the organic phase with a saturated solution of NaCl, and separating the organic phase; concentrating the obtained organic phase, purifying the resulting solid by column chromatography (dichloromethane/ethyl acetate, 3:1 v/v) to isolate a compound pyridine macrocycle; subsequently, adding sequentially 1.8 g of pyridine macrocycle and 1.5 g of 2,4-dinitrochlorobenzene to a 50 mL round-bottomed flask, adding 5 mL of acetone, mixing well the mixture by ultrasonication, and then heating the reactants at reflux overnight; after the reaction is completed, removing the solvent by rotary evaporation, adding a large amount of ethyl acetate, performing suction filtration, dissolving the filter cake in acetonitrile, performing suction filtration, drying the filtrate by rotary evaporation, dissolving the residue in a small amount of methanol, adding ethyl acetate, stirring the mixture for 1-4 h, and performing suction filtration to obtain an intermediate product; adding 0.5 g of the intermediate product to 1 mL of ethanol, then adding 3 mL of water, mixing well the mixture, then adding 3.5 g of p-bromophenylamine, and refluxing the mixture under nitrogen atmosphere for 1-2 days; after the reaction is completed, cooling the mixture to room temperature, adding ethyl acetate, performing suction filtration, and removing the solvent by rotary evaporation; adding a small amount of acetone to dissolve the solid, then adding a large amount of ethyl acetate to precipitate a solid, and performing suction filtration to obtain the target product.

The present disclosure also provides use of the monomer of supramolecular macrocyclic and cage-like molecule-like compound based on biphen[n]arene described above in preparing the supramolecular macrocyclic and cage-like molecule compound based on biphen[n]arene and the derivative compounds described above.

The present disclosure also provides use of the supramolecular macrocyclic and cage-like molecule compound based on biphen[n]arene and derivative compound thereof described above in materials, environment and biology.

According to an embodiment of the present disclosure, the macrocyclic compound based on biphen[n]arene is used as an adsorptive separation material for trimethylbenzene isomer or for the recognition of an ammonium cationic compound.

According to an embodiment of the present disclosure, the macrocyclic compound based on biphen[n]arene is used for the adsorptive separation of cyclohexane and chlorocyclohexane.

According to an embodiment of the present disclosure, the macrocyclic and cage-like molecule based on biphen[n]arene and derivative compound thereof is used for the recognition of a toxic cationic derivatives such as purpurine molecule and o-phenanthroline.

According to an embodiment of the present disclosure, the derivative macrocyclic arene is used as a phosphorescent luminescent material.

The present disclosure is described in more detail as follows:

In the synthesis and derivatization of the macrocyclic and cage-like molecule based on biphen[n]arene, by using a bis-(2,4-dialkoxyphenyl)arene and an aldehyde compound as starting materials, and using a Lewis acid as a catalyst in a haloalkane solvent, a supramolecular macrocyclic and cage-like molecule are synthesized, and a water-soluble derivative macrocyclic arene and cage-like molecule are obtained by further modification.

The synthesis and derivatization of the macrocyclic and cage-like molecule based on biphen[n]arene comprise the following steps:

step 1, synthesis of the monomer of supramolecular macrocyclic and cage-like molecule-like compound based on biphen[n]arene;

step 2, synthesis of a macrocyclic and cage-like molecule based on biphen[n]arene; and

step 3, derivatization of the supramolecular macrocyclic and cage-like molecule based on biphen[n]arene.

I. A synthesis method for the supramolecular macrocyclic and cage-like molecule based on biphen[n]arene monomers of step 1 is as follows:

[1] Preparation of a Monomer of Supramolecular Macrocycle Based on biphen[n]arene

dissolving a dibromide or an iodide and 2,4-dimethoxyphenylboronic acid in an aqueous solution of dioxane (dioxane:water=5:1), then adding tetrakis(triphenylphosphine)palladium catalyst and sodium carbonate, and stirring the mixture at reflux overnight; after the reaction is completed, cooling the reaction mixture to room temperature, removing the solvent by rotary evaporation, dissolving the residue in dichloromethane, and washing the resulting solution with water three times; drying the organic layer over anhydrous Na₂SO₄ and removing the solvent again by rotary evaporation, and preparing the residue for column chromatography to isolate the monomer:

[2] Preparation of a Monomer of Cage-Like Molecule

dissolving a tribromide and 2,4-dimethoxyphenylboronic acid in an aqueous solution of dioxane (dioxane:water=5:1), then adding tetrakis(triphenylphosphine)palladium catalyst and sodium carbonate, and stirring the mixture at reflux overnight; after the reaction is completed, cooling the reaction mixture to room temperature, removing the solvent by rotary evaporation, dissolving the residue in dichloromethane, and washing the resulting solution with water three times; drying the organic layer over anhydrous Na₂SO₄ and removing the solvent again by rotary evaporation, and preparing the residue for column chromatography to isolate the monomer:

[3] Preparation of a Macrocyclic Monomer with Dibutoxyl or 4-methoxy-2-(5-bromo-n-pentyloxy) Side Chain

1) Synthesis of a Macrocyclic Monomer with Dibutoxy Side Chain

adding excessively n-butyl bromide to a three-necked flask and heating n-butyl bromide at reflux, starting dissolving 4-bromo-resorcinol in acetonitrile and adding dropwise the resulting solution to the reaction system, and allowing the reaction system to react overnight; after the reaction is completed, stopping the heating, and filtering the mixture to remove potassium carbonate; concentrating the reaction solution by rotary evaporation, and subjecting the residue to column chromatography to isolate 4-bromo-2,4-dibutoxybenzene reaction product; subsequently, dissolving completely 4-bromo-1,3-dibutoxybenzene in an aqueous solution of 1,4-dioxane (dioxane:water=5:1), then adding 4,4′-biphenyldiboronic acid, tetrakis(triphenylphosphine)palladium and sodium carbonate, and heating the mixed system to 100° C. and refluxing overnight; after the reaction is completed, cooling the reaction mixture to room temperature, removing the solvent by rotary evaporation, dissolving the residue in dichloromethane, and washing the resulting solution with water three times; drying the organic layer over anhydrous Na₂SO₄ and removing the solvent again by rotary evaporation, and preparing the residue for column chromatography to isolate the monomer;

2) Synthesis of a Macrocyclic Monomer with 4-methoxy-2-(5-bromo-n-pentyloxy) Side Chain

adding excessively 1,5-dibromopentane to a three-necked flask and heating 1,5-dibromopentane at reflux, starting dissolving 2-bromo-5-methoxyphenol in acetonitrile and adding dropwise the resulting solution to the reaction system, and allowing the reaction system to react overnight; after the reaction is completed, stopping the heating, and filtering the mixture to remove potassium carbonate; concentrating the reaction solution by rotary evaporation, and subjecting the residue to column chromatography to isolate 4-methoxy-2-(5-bromo-n-pentyloxy)bromobenzene reaction product; subsequently, dissolving completely 4-methoxy-2-(5-bromo-n-pentyloxy)bromobenzene in an aqueous solution of 1,4-dioxane (dioxane:water=5:1), then adding 4,4′-biphenyldiboronic acid, tetrakis(triphenylphosphine)palladium and sodium carbonate, and heating the mixed system to 100° C. and refluxing overnight; after the reaction is completed, cooling the reaction mixture to room temperature, removing the solvent by rotary evaporation, dissolving the residue in dichloromethane, and washing the resulting solution with water three times; drying the organic layer over anhydrous Na₂SO₄ and removing the solvent again by rotary evaporation, and preparing the residue for column chromatography to isolate the monomer.

II. A synthesis method for the macrocyclic and cage-like molecule based on biphen[n]arene of step 2 is as follows:

[1] Synthesis of a Supramolecular Macrocycle with Trimer or Higher Degree of Polymerization from a Molecule Having a Linear Structure:

using a bis-(2,4-dialkoxyphenyl)arene having a linear structure and an aldehyde reactant as starting materials and a haloalkane as a solvent, adding a Lewis acid catalyst after the starting materials are dissolved, monitoring the reaction by thin-layer chromatography (TLC), quenching the reaction with saturated aqueous sodium bicarbonate solution after the reaction is completed, washing the mixture with saturated aqueous sodium chloride solution, drying the mixture over anhydrous sodium sulfate, and subjecting the resulting mixture to silica gel column chromatography to isolate a ring formation product with trimer or higher degree of polymerization; wherein the halohydrocarbon solvent is at least one selected from: dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, and monobromodecane; the aldehyde reactant is selected from paraformaldehyde and isobutyraldehyde;

[2] Preparation of a Dimeric Macrocycle from a V-Shaped Molecule:

using a bis-(2,4-dialkoxyphenyl)arene having a V-shaped structure and an aldehyde reactant as starting materials and a haloalkane as a solvent, adding a Lewis acid catalyst after the starting materials are dissolved, monitoring the reaction by thin-layer chromatography (TLC), quenching the reaction with saturated aqueous sodium bicarbonate solution after the reaction is completed, washing the mixture with saturated aqueous sodium chloride solution, drying the mixture over anhydrous sodium sulfate, and subjecting the resulting mixture to silica gel column chromatography to isolate a dimeric ring formation product; wherein the halohydrocarbon solvent is at least one selected from: dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, and monobromodecane; the aldehyde reactant is selected from paraformaldehyde and isobutyraldehyde;

[3] Synthesis of a Cage-Like Molecule from tris-(2,4-dialkoxyphenyl)arene:

using a tris-(2,4-dialkoxyphenyl)arene and paraformaldehyde or isobutyraldehyde as starting materials (the molar ratio of tris-(2,4-dialkoxyphenyl)arene to paraformaldehyde or isobutyraldehyde is about 1:5) and a haloalkane as a solvent, adding a Lewis acid catalyst after the starting materials are dissolved, monitoring the reaction by thin-layer chromatography (TLC), quenching the reaction with saturated aqueous sodium bicarbonate solution after the reaction is completed, washing the mixture with saturated aqueous sodium chloride solution, drying the mixture over anhydrous sodium sulfate, and subjecting the resulting mixture to silica gel column chromatography to isolate a cage-like molecule compound product; wherein the halohydrocarbon solvent is at least one selected from: dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, and monobromodecane; the aldehyde reactant is selected from paraformaldehyde and isobutyraldehyde;

[4] Preparation of a Supramolecular Macrocyclic Compound in which a Macrocycle has Different Units by Regulating Proportions of Different Monomer Molecules to Achieve Copolymerization of the Different Monomers:

adding two bis-(2,4-dialkoxyphenyl)arenes to a reaction flask in a molar ratio of 1:5, then adding a paraformaldehyde in an amount that is twice the total amount of substance of the two derivatives, adding a Lewis acid catalyst after dissolution in a haloalkane, monitoring the reaction by thin-layer chromatography (TLC), quenching the reaction with saturated aqueous sodium bicarbonate solution after the reaction is completed, washing the mixture with saturated aqueous sodium chloride solution, drying the mixture over anhydrous sodium sulfate, and subjecting the resulting mixture to silica gel column chromatography to isolate a copolymeric three-membered macrocyclic compound; the halohydrocarbon solvent is at least one selected from: dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, and monobromodecane.

III. A derivatization method for the macrocyclic and cage-like molecule based on biphen[n]arene of step 3 is as follows:

[1] Synthesis of a Hydroxyl Compound

adding a macrocyclic compound to a round-bottomed flask, dissolving the macrocyclic compound with dichloromethane, and adding 20 equivalents of boron tribromide compound to the reaction system; after 1 day of reaction, adding dropwise the reaction mixture to a mixture of ice and water to precipitate a light purple powder; and performing suction filtration to obtain a hydroxyl macrocyclic product:

[2] Synthesis of a Carboxylic Acid Water-Soluble Macrocyclic and Cage-Like Molecule

in a round-bottomed flask, dissolving the hydroxyl macrocyclic compound in acetonitrile, then adding K₂CO₃, refluxing the mixture for 2 h, then adding ethyl bromoacetate, and refluxing the mixture for another 48 h; after the reaction is completed, cooling the reaction mixture to room temperature, filtering, and washing the mixture with dichloromethane multiple times, removing the solvent by vacuum rotary evaporation, adding a small amount of dichloromethane so that the solid is just dissolved, then adding a large amount of petroleum ether, with a large amount of solid subsequently precipitated, and performing suction filtration under reduced pressure to obtain the desired product; dissolving the product in a mixed solution of 50 mL of THF and 20 mL of an aqueous solution of sodium hydroxide (a mass concentration of 20%) and stirring the mixture at reflux for 10 h; removing THF by rotary evaporation, then adding 20 mL of water, then adding hydrochloric acid for acidification until a pH test paper shows weak acidity, with a large amount of solid precipitated, and performing suction filtration to obtain a carboxylic acid derivative macrocyclic and cage-like molecule; then adding gradually the carboxylic acid derivative macrocyclic and cage-like molecule to an alkali solution to obtain a carboxylate salt water-soluble macrocyclic and cage-like molecule compound of the corresponding alkali;

[3] Synthesis of a Sulfonated Water-Soluble Macrocycle

in a 100 mL round bottom flask, dissolving the hydroxyl macrocyclic compound in acetone, adding K₂CO₃, stirring the mixture at reflux for 2 h, then adding 1 equivalent of propane sultone, and stirring the mixture at reflux for another 3 days; after the reaction is stopped, cooling the mixture to room temperature, performing suction filtration, washing the filter cake twice with acetone, dissolving the obtained filter cake in water, performing purification by dialysis with a dialysis bag to removing potassium carbonate by about one week of dialysis; and finally, concentrating the aqueous solution in the dialysis bag by rotary evaporation to obtain a sulfonated water-soluble macrocyclic product;

[4] Special Derivatization of Some Macrocyclic Compounds

[1] Synthesis of a Carbazole Derivative Macrocycle:

\textcircled{1} Monomer Modification, and then Ring Closing:

dissolving bis-(2,4-dialkoxyphenyl)carbazole and 2 equivalents of p-dibromobenzene or methyl 5-bromoisophthalate in N,N-dimethylacetamide, adding 1 equivalent of copper(I) iodide and 6 equivalents of potassium carbonate, heating the mixture to 180° C. under nitrogen (argon) atmosphere for 24 h of reaction, cooling the mixture to room temperature after the reaction is completed, pouring the product into a saturated aqueous solution of NaCl, performing extraction with dichloromethane three times, performing drying over anhydrous sodium sulfate, and performing purification by column chromatography to obtain a product in the form of a white solid; dissolving the monomer above and 3 equivalents of paraformaldehyde in dichloromethane, adding boron trifluoride diethyl ether catalyst, and monitoring the reaction by thin-layer chromatography (TLC); after the reaction is completed, quenching the reaction with saturated aqueous sodium bicarbonate solution, washing the mixture with saturated aqueous sodium chloride solution, drying the mixture over anhydrous sodium sulfate, and subjecting the resulting mixture to silica gel chromatography to isolate a carbazole three-membered ring macrocycle product modified by p-dibromobenzene or methyl 5-bromoisophthalate;

\textcircled{2} Ring Closing, and then Modification:

dissolving bis-(2,4-dialkoxyphenyl)carbazole and 3 equivalents of paraformaldehyde in dichloromethane, adding 2 equivalents of boron trifluoride diethyl ether catalyst, monitoring the reaction by thin-layer chromatography (TLC), quenching the reaction with saturated aqueous sodium bicarbonate solution after the reaction is completed, washing the mixture with saturated aqueous sodium chloride solution, drying the mixture over anhydrous sodium sulfate, and subjecting the resulting mixture to silica gel chromatography to isolate a carbazole-modified three-membered ring product; dissolving a mixture of the carbazole-modified three-membered ring, 2 equivalents of p-dibromobenzene or methyl 5-bromoisophthalate, 1 equivalent of copper(I) iodide and 6 equivalents of potassium carbonate in N,N-dimethylacetamide, heating the resulting solution to 180° C. under nitrogen (argon) atmosphere for 24 h of reaction, then cooling the reaction solution to room temperature, pouring the product into a saturated aqueous solution of NaCl, performing extraction with dichloromethane three times, performing drying over anhydrous sodium sulfate, and performing purification by column chromatography to obtain a three-membered ring product modified by p-dibromobenzene or methyl 5-bromoisophthalate;

[2] Synthesis of a Pyridine Derivative Macrocycle:

\textcircled{1} Monomer Modification and then Ring Closing:

in a 100 mL three-necked flask, adding 2.4 g of 3,5-dibromopyridine and 3.6 g of 2,4-dimethoxyphenylboronic acid to a beaker in a molar ratio of 1:2, then adding 2.1 g of anhydrous sodium carbonate and 0.4 g of tetrakis(triphenylphosphine)palladium, using 80 mL of 1,4-dioxane and 20 mL of water as solvents, heating the resulting mixture at reflux in a 110° C. oil bath overnight, after the reaction is completed, evaporating sequentially the solvents, performing extraction with water and dichloromethane, and performing drying over anhydrous sodium sulfate, and separating out the organic phase; concentrating the obtained organic phase, and subjecting the residue to column chromatography for purification to isolate 3,5-bis(2,4-dimethoxyphenyl)pyridine in the form of a white solid; adding sequentially 1.7 g of 3,5-bis-(2,4-dimethoxyphenyl)pyridine monomer and 1.5 g of 2,4-dinitrochlorobenzene to a 50 mL round-bottomed flask, adding 5 mL of acetone, mixing well the mixture by ultrasonication, then heating the reactants at reflux overnight, after the reaction is completed, removing the solvent by rotary evaporation, adding a large amount of ethyl acetate, performing suction filtration, adding the filter cake to an acetonitrile solution, performing suction filtration, collecting the filtrate, concentrating the filtrate by rotary evaporation, adding a small amount of methanol to completely dissolve the residue, adding ethyl acetate, stirring the mixture for 1-4 h, and performing suction filtration to obtain an intermediate product; adding 0.5 g of the intermediate product to a 50 mL round-bottomed flask, adding 1 mL of ethanol, then adding 3 mL of water, mixing well the mixture, then adding 3.5 g of p-bromophenylamine, heating the mixture at reflux under nitrogen atmosphere for 1-2 days, cooling the mixture to room temperature, then adding ethyl acetate, and performing suction filtration; adding ethanol to the filtrate, removing the solvent by rotary evaporation, then adding a small amount of acetone to dissolve the solid, then adding a large amount of ethyl acetate, and performing suction filtration to obtain a modified pyridine arene derivative monomer; then weighing 1 g of the derivative arene monomer and 0.3 g of paraformaldehyde, pouring 150 mL of dichloromethane solvent for dissolution, adding 1.5 equivalents of boron trifluoride diethyl ether catalyst while stirring, monitoring the reaction by thin-layer chromatography, after 30 min of reaction, adding 100 mL of a saturated solution of sodium bicarbonate to quench the reaction, washing the mixture with 50 mL of a saturated solution of sodium chloride, drying the mixture over anhydrous sodium sulfate, and subjecting the resulting mixture to silica gel chromatography to obtain the target product;

\textcircled{2} Ring Closing, and then Modification:

dissolving 3,5-bis(2,4-dimethoxyphenyl)pyridine compound (3.52 g, 10 mmol) in 100 mL of dichloromethane, and adding paraformaldehyde (0.45 g, 15 mmol); stirring the mixture for a few minutes until complete dissolution is achieved, adding boron trifluoride diethyl ether catalyst to the mixture so that the reaction mixture gradually turns from colorless to light purple, stirring the mixture at room temperature for 25 min, monitoring the reaction by TLC, and quenching the reaction with a saturated solution of sodium bicarbonate when the starting materials are substantially consumed; separating out the organic phase, washing the organic phase once with water, and finally extracting the organic phase with a saturated solution of NaCl, and separating the organic phase; concentrating the obtained organic phase, purifying the resulting solid by column chromatography (dichloromethane/ethyl acetate, 3:1 v/v) to isolate a compound pyridine macrocycle;

subsequently, adding sequentially 1.8 g of pyridine macrocycle and 1.5 g of 2,4-dinitrochlorobenzene to a 50 mL round-bottomed flask, adding 5 mL of acetone, mixing well the mixture by ultrasonication, and then heating the reactants at reflux overnight; after the reaction is completed, removing the solvent by rotary evaporation, adding a large amount of ethyl acetate, performing suction filtration, dissolving the filter cake in acetonitrile, performing suction filtration, drying the filtrate by rotary evaporation, dissolving the residue in a small amount of methanol, adding ethyl acetate, stirring the mixture for 1-4 h, and performing suction filtration to obtain an intermediate product; adding 0.5 g of the intermediate product to 1 mL of ethanol, then adding 3 mL of water, mixing well the mixture, then adding 3.5 g of p-bromophenylamine, and refluxing the mixture under nitrogen atmosphere for 1-2 days; after the reaction is completed, cooling the mixture to room temperature, adding ethyl acetate, performing suction filtration, and removing the solvent by rotary evaporation; adding a small amount of acetone to dissolve the solid, then adding a large amount of ethyl acetate to precipitate a solid, and performing suction filtration to obtain the target product.

The synthesis method for the macrocyclic and cage-like based on biphen[n]arene hosts disclosed in the present disclosure mainly comprises: under catalysis of a Lewis acid, performing a one-pot process with a bis-(2,4-dialkoxyphenyl)arene (selected from benzene, biphenyl, terphenyl, quaterphenyl, naphthalene, anthracene, anthraquinone, pyrene, porphyrin, fluorenone, carbazole, benzothiadiazole, styrene, trans-stilbene, tetrafluorobenzene, tetraphenylene, diphenylpropanedione, boron-dipyrromethene (BODIPY) fluorophore, and the like) or a tris-(2,4-dialkoxyphenyl)arene (benzene or tribenzobenzene) and paraformaldehyde (formaldehyde or isobutyraldehyde) to obtain a series of corresponding novel macrocyclic or cage-like compounds with high yield. The configuration of the monomer greatly influences the type of ring formation; a linear (at an included angle of 180°) monomer mainly forms a three- or higher-membered ring, a V-shaped (at an included angle of 120°, 90° or 60°) monomer mainly forms a two-membered ring, and a tris-(2,4-dialkoxyphenyl)arene (three-claw-shaped) monomer forms a cage-like compound. The synthesis of macrocycles based on biphen[n]arene is also achieved in a modular manner by copolymerization of different monomers. In addition, under the action of a demethylating reagent, the methyl groups in these macrocycles are easy to remove to obtain a hydroxyl macrocyclic arene (a quaterphenyl three-membered ring, a tetraphenylethylene two-membered ring, a naphthalene two-membered ring, or a tribenzobenzene cage-like molecule). The macrocyclic compounds are mainly used for the adsorptive separation materials of trimethylbenzene isomer or the recognition of an ammonium cationic compound. The cage-like compound is used for the adsorptive separation of cyclohexane and chlorocyclohexane. A variety of water-soluble macrocyclic derivatives can be obtained by further modification. Among these derivatives, macrocyclic and cage-like water-soluble derivative compounds based on biphen[n]arene are mainly used for recognition of toxic cationic derivatives such as purpurine molecules and o-phenanthroline. Besides, various functional groups introduced into the framework enable the macrocyclic arene to show excellent adsorptive separation performance and photophysical properties, providing a structural foundation for the application of macrocyclic compounds based on biphen[n]arene in the fields of materials, photoelectricity, etc., wherein the specially derivative macrocyclic arenes are mainly used for phosphorescent luminescent materials.

The present disclosure has the following advantages: the starting materials are commercially available, the synthesis is simple and convenient (one-pot process), the yield is high, and the framework and side chain can be modified very conveniently, so that the present disclosure has great scientific research value in the fields of macrocyclic and supramolecular chemistry, and has prospects for wide application in the aspects of gas adsorption and separation, performance improvement of luminescent materials, adsorption of water-soluble toxic substances, and the like.

### Definitions and Description

In this application,

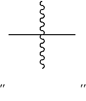

indicates a connection site unless otherwise specified.

### BRIEF DESCRIPTION OF THE DRAWINGS

FIG. 1 shows the adsorption selectivity of macrocycle 1 for mesitylene and 1,2,4-trimethylbenzene;

FIG. 2 shows the adsorption of mesitylene and 1,2,4-trimethylbenzene by macrocycle 1 over time;

FIG. 3 shows a nuclear magnetic resonance spectrum of macrocycle 1 after 12 h of adsorption in a mixed vapor of 1,2,4-trimethylbenzene and mesitylene (v/v=1:1) for comparison;

FIG. 4 shows a nuclear magnetic resonance spectrum of macrocycle 1 with integration values after 12 h of adsorption in a mixed vapor of 1,2,4-trimethylbenzene and mesitylene (v/v=1:1);

FIG. 5 shows structures of guest molecules 1-4;

FIG. 6 shows a 1:1 nuclear magnetic resonance spectrum (CDCl₃, 298 K, 5 mmol/L) of 1⁺-BHArF and macrocyclic arene 21 for comparison; (A) guest 1⁺-BHArF alone; (B) macrocyclic arene 21+1⁺-BHArF; (C) host macrocyclic arene 21 alone;

FIG. 7 shows a 1:1 nuclear magnetic resonance spectrum (CDCl₃, 298 K, 5 mmol/L) of 1⁺-BHArF and macrocyclic arene 17 for comparison; (A) guest 1⁺-BHArF alone; (B) macrocyclic arene 17+1⁺-BHArF; (C) host macrocyclic arene 17 alone;

FIG. 8 shows a 1:1 nuclear magnetic resonance spectrum (CDCl₃, 298 K, 5 mmol/L) of 2⁺-BHArF and macrocyclic arene 21 for comparison; (A) guest 2⁺-BHArF alone; (B) macrocyclic arene 21+2⁺-BHArF; (C) host macrocyclic arene 21 alone;

FIG. 9 shows a 1:1 nuclear magnetic resonance spectrum (CDCl₃, 298 K, 5 mmol/L) of 2⁺-BHArF and macrocyclic arene 17 for comparison; (A) guest 2⁺-BHArF alone; (B) macrocyclic arene 17+2⁺-BHArF; (C) host macrocyclic arene 17 alone;

FIG. 10 shows a 1:1 nuclear magnetic resonance spectrum (CDCl₃, 298 K, 5 mmol/L) of 3⁺-BHArF and macrocyclic arene 21 for comparison; (A) guest 3⁺-BHArF alone; (B) macrocyclic arene 21+3⁺-BHArF; (C) host macrocyclic arene 21 alone;

FIG. 11 shows a 1:1 nuclear magnetic resonance spectrum (CDCl₃, 298 K, 5 mmol/L) of 3⁺-BHArF and macrocyclic arene 17 for comparison; (A) guest 3⁺-BHArF alone; (B) macrocyclic arene 17+3⁺-BHArF; (C) host macrocyclic arene 17 alone;

FIG. 12 shows a 1:1 nuclear magnetic resonance spectrum (CDCl₃, 298 K, 5 mmol/L) of 4⁺-BHArF and macrocyclic arene 21 for comparison; (A) guest 4⁺-BHArF alone; (B) macrocyclic arene 21+4-BHArF; (C) host macrocyclic arene 21 alone;

FIG. 13 shows a 1:1 nuclear magnetic resonance spectrum (CDCl₃, 298 K, 5 mmol/L) of 4⁺-BHArF and macrocyclic arene 17 for comparison; (A) guest 4⁺-BHArF alone; (B) macrocyclic arene 17+4⁺-BHArF; (C) host macrocyclic arene 17 alone;

FIG. 14 shows a fit nuclear magnetic resonance titration curve of macrocyclic arene 17 and guest 1⁺-BHArF, and a non-linear fit curve of macrocyclic arene 17 (0.2 mM) and guest 1⁺-BHArF in CDCl₃ (guest concentrations: 0, 0.04, 0.14, 0.32, 0.54, 0.88, 1.50, 2.54, 3.39, 4.71, 6.41, and 8.17 mM);

FIG. 15 shows changes after 4 h of adsorption in a mixed vapor of chlorocyclohexane and cyclohexane (v/v=1:1) by cage-like molecule 27, and nuclear magnetic resonance spectra of the host and guests for comparison;

FIG. 16 shows a nuclear magnetic resonance spectrum of cage-like molecule 27 with integration values after 4 h of adsorption in a mixed vapor of chlorocyclohexane and cyclohexane (v/v=1:1);

FIG. 17 shows a 1:1 nuclear magnetic resonance spectrum (D₂O, 298 K, 5 mmol/L) of paraquat 1²⁺ and macrocycle 35 for comparison; (A) guest 1²⁺ alone; (B) macrocycle 35+1²⁺; (C) host macrocycle 35 alone;

FIG. 18 shows a 1:1 nuclear magnetic resonance spectrum (D₂O, 298 K, 5 mmol/L) of 2²⁺ and macrocycle for comparison; (A) guest 2²⁺ alone; (B) macrocycle 35+2²⁺; (C) host macrocycle 35 alone;

FIG. 19 shows a 1:1 nuclear magnetic resonance spectrum (D₂O, 298 K, 5 mmol/L) of purpurine guest and water-soluble cage-like molecule 36 for comparison; (A) host water-soluble cage-like molecule 36 alone; (B) macrocycle 36+purpurine guest; (C) purpurine guest alone;

FIG. 20 shows a 1:1 nuclear magnetic resonance spectrum (D₂O, 298 K, 5 mmol/L) of derivative o-phenanthroline guest and water-soluble cage-like molecule 36 for comparison; (A) host water-soluble cage-like molecule 36 alone; (B) macrocycle 36+derivative o-phenanthroline guest; (C) derivative o-phenanthroline guest alone;

FIG. 21 shows fit ITC curves of water-soluble cage-like molecule 36 for purpurine molecule (left) and o-phenanthroline cationic derivative molecule (right);

FIG. 22 shows fluorescence excitation and emission spectra of three-membered ring macrocycle product 39; and

FIG. 23 shows a time-resolved spectrum (lifetime image) of three-membered ring macrocycle product 39.

In the above description of the drawings, unless otherwise specified, a 1:1 nuclear magnetic resonance spectrum for comparison refers to a nuclear magnetic resonance spectrum obtained by adding a host and a guest to a deuterated reagent in an amount-of-substance ratio of 1:1 for comparison with nuclear magnetic resonance spectra of the host alone and the guest alone to observe changes of peaks in position in the nuclear magnetic resonance spectra of the host and guest.

### DETAILED DESCRIPTION

The synthesis, derivatization and use of the biphenyl arene macrocyclic and cage-like molecule involved in the present disclosure are described in detail below with reference to the drawings. However, the present disclosure is not limited to the following examples. In order to achieve sufficient public understanding of the present disclosure, details are provided in preferred embodiments of the present disclosure. It is hereby stated that the starting materials used in the present disclosure are all commercially available, the monomers that have not been reported are claimed and synthesis methods thereof are disclosed, and the monomers and rings that have been reported are set forth in the specification with CAS numbers and references indicated.

In the expression “solventA:solvent B=C:D” referred to in the following examples, C:D refers to a volume ratio, unless otherwise specified.

### Example 1

In the synthesis and derivatization of macrocycles based on biphen[n]arene and cage-like molecules, Suzuki coupling reactions of a dibromo (iodo) or tribromo starting material and a substituted phenylboronic acid are performed to obtain bis-(2,4-dialkoxyphenyl)arene monomers (designated M1-M27), and then the obtained monomers and an aldehyde compound are reacted as starting materials in a haloalkane solvent under catalysis by a Lewis acid to obtain the supramolecular macrocyclic and cage-like molecule based on biphen[n]arene; by further derivatization and modification, water-soluble macrocyclic arenes and cage-like molecules (designated 1-41) can be obtained. The synthesis and derivatization of the macrocyclic and cage-like molecule based on biphen[n]arene comprise the following steps:

step 1, synthesis of macrocyclic and cage-like molecule based on biphen[n]arene monomeric compounds;

step 2, synthesis of a macrocyclic and cage-like molecule based on biphen[n]arene; and

step 3, derivatization of the macrocyclic and cage-like molecule based on biphen[n]arene.

I. A synthesis method for the macrocyclic and cage-like molecule based on biphen[n]arene monomeric compounds of step 1 is as follows:

[1] Preparation of a Macrocyclic Monomer with Methoxy Side Chain

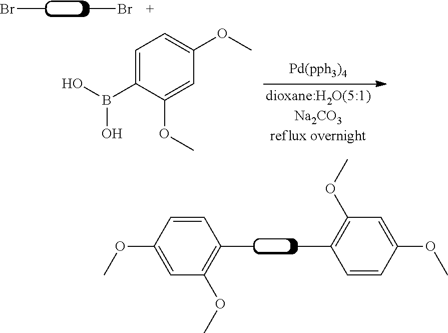

The macrocyclic monomer prepared are shown in the table below:

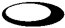

| | |
|---|---|
| 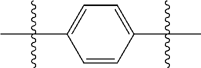 [SMILES: *c1ccc(*)cc1] | M1 |
| 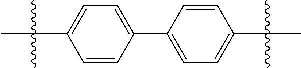 [SMILES: *c1ccc(-c2ccc(*)cc2)cc1] | M2 |
| 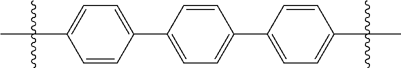 [SMILES: *c1ccc(-c2ccc(-c3ccc(*)cc3)cc2)cc1] | M3 |
| 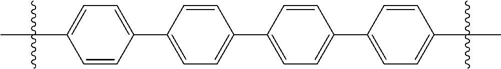 [SMILES: *c1ccc(-c2ccc(-c3ccc(-c4ccc(*)cc4)cc3)cc2)cc1] | M4 |
| 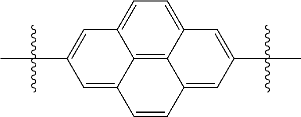 [SMILES: *c1cc2ccc3cc(*)cc4ccc(c1)c2c34] | M5 |
| 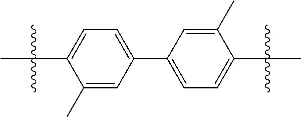 [SMILES: *c1ccc(-c2ccc(*)c(C)c2)cc1C] | M6 |
| 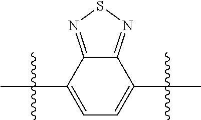 [SMILES: *c1ccc(*)c2nsnc12] | M7 |
| 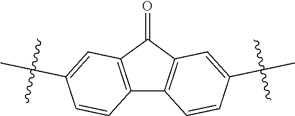 [SMILES: *c1ccc2c(c1)C(=O)c1cc(*)ccc1-2] | M8 |
| 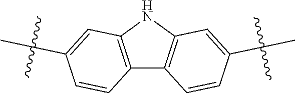 [SMILES: *c1ccc2c(c1)[nH]c1cc(*)ccc12] | M9 |
| 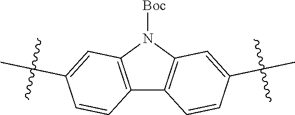 [SMILES: *c1ccc2c3ccc(*)cc3n(C(=O)OC(C)(C)C)c2c1] | M10 |
| 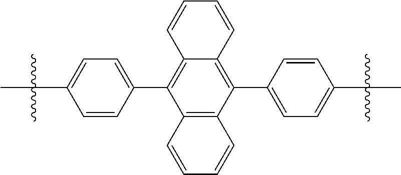 [SMILES: *c1ccc(-c2c3ccccc3c(-c3ccc(*)cc3)c3ccccc23)cc1] | M11 |
| 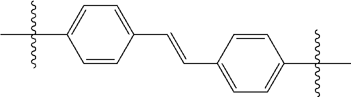 [SMILES: *c1ccc(/C=C/c2ccc(*)cc2)cc1] | M12 |
| 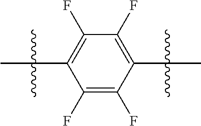 [SMILES: *c1c(F)c(F)c(*)c(F)c1F] | M13 |
| 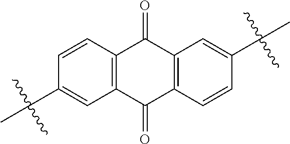 [SMILES: *c1ccc2c(c1)C(=O)c1ccc(*)cc1C2=O] | M14 |
| 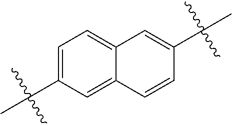 [SMILES: *c1ccc2cc(*)ccc2c1] | M15 |
| 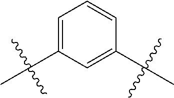 [SMILES: *c1cccc(*)c1] | M16 |
| 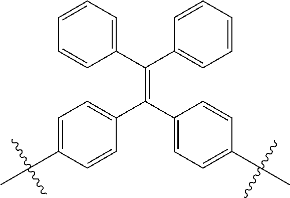 [SMILES: *c1ccc(C(=C(c2ccccc2)c2ccccc2)c2ccc(*)cc2)cc1] | M17 |
| 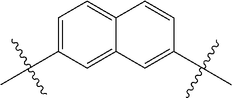 [SMILES: *c1ccc2ccc(*)cc2c1] | M18 |
| 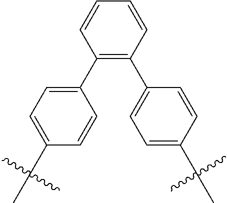 [SMILES: *c1ccc(-c2ccccc2-c2ccc(*)cc2)cc1] | M19 |
| 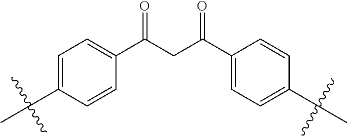 [SMILES: *c1ccc(C(=O)CC(=O)c2ccc(*)cc2)cc1] | M20 |
| 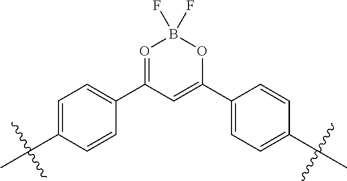 | M21 |
| 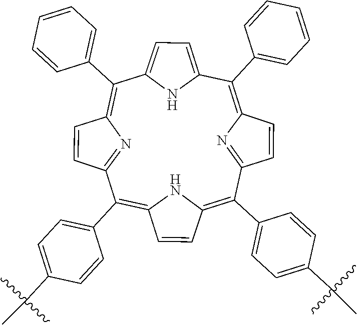 [SMILES: *c1ccc(-c2c3nc(c(-c4ccccc4)c4ccc([nH]4)c(-c4ccccc4)c4nc(c(-c5ccc(*)cc5)c5ccc2[nH]5)C=C4)C=C3)cc1] | M22 |

(1) Preparation of Monomer M1

2.3 g of p-dibromobenzene (CAS: 106-37-6) was completely dissolved in an aqueous solution of 1,4-dioxane (dioxane:water=5:1), and then 3.6 g of 2,4-dimethoxyphenylboronic acid, 0.4 g of tetrakis(triphenylphosphine)palladium and 4.2 g of sodium carbonate were added. The mixed system was heated to 100° C. and refluxed overnight. After the reaction was completed, the reaction mixture was cooled to room temperature and concentrated by rotary evaporation to remove the solvent. The residue was dissolved in dichloromethane, and the resulting solution was washed with water three times. The organic layer was dried over anhydrous Na₂SO₄ and concentrated again by rotary evaporation. The residue was prepared for column chromatography to isolate a monomer M1.

¹H NMR (400 MHz, CDCl₃) δ 7.53 (4H), 7.30 (2H), 6.58 (2H), 6.57 (4H), 3.86 (6H), 3.82 (6H); HRMS (ESI): m/z calcd. for C₂₂H₂₃O₄⁺ [M]⁺, 351.1591; found: 351.1585.

(2) Preparation of Monomer M2

A monomer M2 was synthesized according to the literature (Vila, Carlos; Cembellin, Sara; Hornillos, Valentin; Giannerini, Massim o; Fañanás-Mastral, Martin; Feringa, Ben L. Chemistry—A European Journal, 2015, 21, 44, 15520-15524.).

(3) Preparation of Monomer M3

3.7 g of 4,4″-dibromo-p-terphenyl (CAS: 17788-94-2) was completely dissolved in an aqueous solution of 1,4-dioxane (dioxane:water=5:1), and then 3.6 g of 2,4-dimethoxyphenylboronic acid, 0.4 g of tetrakis(triphenylphosphine)palladium and 4.2 g of sodium carbonate were added. The mixed system was heated to 100° C. and refluxed overnight. After the reaction was completed, the reaction mixture was cooled to room temperature and concentrated by rotary evaporation to remove the solvent. The residue was dissolved in dichloromethane, and the resulting solution was washed with water three times. The organic layer was dried over anhydrous Na₂SO₄ and concentrated again by rotary evaporation. The residue was prepared for column chromatography to isolate a monomer M3.

¹H NMR (400 MHz, CDCl₃) δ 7.72 (4H), 7.68 (4H), 7.61 (4H), 7.32 (2H), 6.60 (4H), 3.87 (6H), 3.84 (6H).

(4) Preparation of Monomer M4

4.4 g of 4,4′″-dibromo-p-quaterphenyl (CAS: 2132-83-4) was completely dissolved in an aqueous solution of 1,4-dioxane (dioxane:water=5:1), and then 3.6 g of 2,4-dimethoxyphenylboronic acid, 0.4 g of tetrakis(triphenylphosphine)palladium and 4.2 g of sodium carbonate were added. The mixed system was heated to 100° C. and refluxed overnight. After the reaction was completed, the reaction mixture was cooled to room temperature and concentrated by rotary evaporation to remove the solvent. The residue was dissolved in dichloromethane, and the resulting solution was washed with water three times. The organic layer was dried over anhydrous Na₂SO₄ and concentrated again by rotary evaporation. The residue was prepared for column chromatography to isolate a monomer M4.

¹H NMR (500 MHz, CDCl₃) δ 7.74 (8H), 7.69 (4H), 7.61 (4H), 7.32 (2H), 6.63-6.59 (4H), 3.87 (6H), 3.84 (6H).

(5) Preparation of Monomer M5

1 g of p-2,7-dibromopyrene (CAS: 102587-98-4) was completely dissolved in an aqueous solution of 1,4-dioxane (dioxane:water=5:1), and then 1.1 g of 2,4-dimethoxyphenylboronic acid, 0.2 g of tetrakis(triphenylphosphine)palladium and 2.1 g of sodium carbonate were added. The mixed system was heated to 100° C. and refluxed overnight. After the reaction was completed, the reaction mixture was cooled to room temperature and concentrated by rotary evaporation to remove the solvent. The residue was dissolved in dichloromethane, and the resulting solution was washed with water three times. The organic layer was dried over anhydrous Na₂SO₄ and concentrated again by rotary evaporation. The residue was prepared for column chromatography to isolate a monomer M5.

¹H NMR (400 MHz, CDCl₃) δ 8.30 (4H), 8.08 (4H), 7.50 (2H), 6.69 (4H), 3.92 (6H), 3.87 (6H); HRMS (ESI): m/z calcd. for C₃₂H₂₇O₄⁺ [M]⁺, 475.1904; found: 475.1904.

(6) Preparation of Monomer M6

1.8 g of p-4,4′-diiodo-3,3′-dimethylbiphenyl (CAS: 7583-27-9) was completely dissolved in an aqueous solution of 1,4-dioxane (dioxane:water=5:1), and then 1.1 g of 2,4-dimethoxyphenylboronic acid, 0.2 g of tetrakis(triphenylphosphine)palladium and 2.1 g of sodium carbonate were added. The mixed system was heated to 100° C. and refluxed overnight. After the reaction was completed, the reaction mixture was cooled to room temperature and concentrated by rotary evaporation to remove the solvent. The residue was dissolved in dichloromethane, and the resulting solution was washed with water three times. The organic layer was dried over anhydrous Na₂SO₄ and concentrated again by rotary evaporation. The residue was prepared for column chromatography to isolate a monomer M6.

¹H NMR (400 MHz, CDCl₃) δ 7.56-7.47 (4H), 7.28 (4H), 7.21-7.11 (2H), 6.67-6.55 (4H), 3.90 (6H), 3.81 (6H), 2.25 (6H).

(7) Preparation of Monomer M7

1.5 g of p-4,4′-dibromo-benzothiadiazole (CAS: 15155-41-6) was completely dissolved in an aqueous solution of 1,4-dioxane (dioxane:water=5:1), and then 1 g of 2,4-dimethoxyphenylboronic acid, 0.2 g of tetrakis(triphenylphosphine)palladium and 2.1 g of sodium carbonate were added. The mixed system was heated to 100° C. and refluxed overnight. After the reaction was completed, the reaction mixture was cooled to room temperature and concentrated by rotary evaporation to remove the solvent. The residue was dissolved in dichloromethane, and the resulting solution was washed with water three times. The organic layer was dried over anhydrous Na₂SO₄ and concentrated again by rotary evaporation. The residue was prepared for column chromatography to isolate a monomer M7.

¹H NMR (500 MHz, CDCl₃) δ 7.70 (2H), 7.53 (2H), 6.82-6.57 (4H), 3.90 (6H), 3.81 (6H); HRMS (ESI): m/z calcd. for C₂₂H₂₀N₂O₄S⁺ [M+H]⁺, 409.1217; found: 409.1218.

(8) Preparation of Monomer M8

1.5 g of p-4,4′-dibromobenzopentanone (CAS: 14348-75-5) was completely dissolved in an aqueous solution of 1,4-dioxane (dioxane:water=5:1), and then 2 g of 2,4-dimethoxyphenylboronic acid, 0.2 g of tetrakis(triphenylphosphine)palladium and 4.2 g of sodium carbonate were added. The mixed system was heated to 100° C. and refluxed overnight. After the reaction was completed, the reaction mixture was cooled to room temperature and concentrated by rotary evaporation to remove the solvent. The residue was dissolved in dichloromethane, and the resulting solution was washed with water three times. The organic layer was dried over anhydrous Na₂SO₄ and concentrated again by rotary evaporation. The residue was prepared for column chromatography to isolate a monomer M8.

¹H NMR (500 MHz, CDCl₃) δ 7.82 (2H), 7.62 (2H), 7.53 (2H), 7.29 (2H), 6.57 (4H), 3.87 (6H), 3.83 (6H).

(9) Preparation of Monomer M9

10 g of 2,7-dibromocarbazole (CAS: 136630-39-2) and 14 g of 2,4-dimethoxyphenylboronic acid were dissolved in 200 mL of a mixed solution of dioxane and water (dioxane:water=5:1), and then 0.8 g of tetrakis(triphenylphosphine)palladium, 6.4 g of sodium carbonate were added to the solution. The reactants were refluxed under N₂ atmosphere for 48 h. The reaction mixture was concentrated in vacuo. The post-concentration solid was dissolved in dichloromethane, and the resulting solution was washed with water. The organic layer was dried over Na₂SO₄ and subjected to column chromatography to isolate a monomer M9.

¹H NMR (500 MHz, CDCl₃) δ 8.04 (2H), 8.06 (2H), 7.55 (4H), 6.64 (4H), 3.90 (6H), 3.86 (6H). HRMS (ESI): m/z calcd. for C₂₈H₂₅NO₄Na⁺ [M+Na]⁺, 440.1977; found: 440.1961.

(10) Preparation of Monomer M10

In a 250 mL flask, 2.63 g of 2,7-dibromocarbazole (CAS: 136630-39-2) was dissolved in 120 mL of anhydrous THF. 2.6 mL of di-tert-butyl dicarbonate and 0.37 g of dimethylaminopyridine are sequentially added. The reaction mixture was stirred at room temperature for 10 h. The solvent was evaporated under reduced pressure. The crude product was completely dissolved in an aqueous solution of 1,4-dioxane (dioxane:water=5:1), and then 2 g of 2,4-dimethoxyphenylboronic acid, 0.2 g of tetrakis(triphenylphosphine)palladium and 4.2 g of sodium carbonate were added. The mixed system was heated to 100° C. and refluxed overnight. After the reaction was completed, the reaction mixture was cooled to room temperature and concentrated by rotary evaporation to remove the solvent. The residue was dissolved in dichloromethane, and the resulting solution was washed with water three times. The organic layer was dried over anhydrous Na₂SO₄ and concentrated again by rotary evaporation. The residue was prepared for column chromatography to isolate a monomer M10.

¹H NMR (500 MHz, CDCl₃) δ 8.46 (2H), 7.96 (2H), 7.51 (2H), 7.37 (2H), 6.60 (4H), 3.88 (6H), 3.84 (6H), 1.72 (9H); HRMS (ESI): m/z calcd. for C₃₃H₃₃NO₆Na⁺ [M+Na]⁺, 539.2308; found: 539.2317.

(11) Preparation of Monomer M11

In a 250 mL flask, 1.4 g of 9,10-bis(4-bromophenyl)anthracene (CAS: 24672-72-8) was completely dissolved in an aqueous solution of 1,4-dioxane, and then 1.3 g of 2,4-dimethoxyphenylboronic acid, 0.4 g of tetrakis(triphenylphosphine)palladium and 3.4 g of Na₂CO₃ were added. The mixed system was heated to 110° C. and refluxed overnight. After the reaction was completed, the reaction mixture was cooled to room temperature and concentrated by rotary evaporation to remove the solvent. The residue was dissolved in dichloromethane, and the resulting solution was washed with water three times. The organic layer was dried over anhydrous Na₂SO₄ and concentrated again by rotary evaporation. The residue was prepared for column chromatography to isolate a monomer M11.

¹H NMR (400 MHz, CDCl₃) δ 7.85 (4H), 7.76 (4H), 7.51 (4H), 7.48-7.43 (2H), 7.36 (4H), 6.66 (4H), 3.92 (6H), 3.91 (6H).

(12) Preparation of Monomer M12

3.0 g of 2,4-dimethoxyphenylboronic acid and 1.7 g of trans-4,4′-dibromostilbene (CAS: 18869-30-2) were dissolved in an aqueous solution of 1,4-dioxane, and 2.1 g of sodium carbonate and 0.2 g of tetrakis(triphenylphosphine)palladium were added. The mixture was stirred at 80° C. at reflux overnight. After the reaction was completed, the reaction mixture was poured into a separatory funnel and washed with water. The organic phase was dried over anhydrous Na₂SO₄ and prepared for column chromatography, and a product monomer M12 was obtained.

¹H NMR (400 MHz, Methylene Chloride-d₂) δ 7.67-7.43 (8H), 7.27 (2H), 7.18 (2H), 6.67-6.49 (4H), 3.83 (12H).

(13) Preparation of Monomer M13

0.6 g of 1,4-dibromotetrafluorobenzene (CAS: 344-03-6), 1.4 g of 2,4-dimethoxyphenylboronic acid, 0.2 g of tetrakis(triphenylphosphine)palladium and 2.1 g of Na₂CO₃ were sequentially added to a two-necked flask, and 12 mL of a mixture of dioxane and water (5:1) was added with a syringe. The flask was placed in an oil bath at a constant temperature of 1200° C., and the reaction mixture was stirred under argon atmosphere for 20 h. After the reaction was completed, the reaction mixture was cooled to room temperature and then extracted with 20 mL of water and 3×10 mL of dichloromethane. The organic phase was dried over anhydrous Na₂SO₄ and purified by rotary evaporation column chromatography to obtain a product monomer M13.

¹H NMR (400 MHz, CDCl₃) δ 7.25-7.18 (2H), 6.72-6.52 (4H), 3.88 (6H), 3.83 (6H); HRMS (ESI): m/z calcd. for C₂₂H₁₈F₄O₄⁺, 423.1214 [M+H]⁺; found: 423.1218.

(14) Preparation of Monomer M14

In a 250 mL round-bottomed flask, 0.7 g of 2,6-dibromoanthraquinone (CAS: 633-70-5), 1.5 g of 2,4-dimethoxyphenylboronic acid and 2 g of sodium carbonate were dissolved in a mixed solvent of 1,4-dioxane (40 mL) and water (10 mL). Then 0.12 g of tetrakis(triphenylphosphine)palladium was added. The solution was heated to 100° C. and stirred at reflux for 24 h. After the reaction was completed, the solvent was evaporated in vacuo, and the mixture was extracted with dichloromethane (20 mL) and washed with distilled water (15 mL). The organic layer was dried over anhydrous sodium sulfate, concentrated, and prepared into a sample. The residue was purified by silica gel column chromatography to obtain an M14 monomer.

¹H NMR (400 MHz, CDCl₃) δ 8.46 (2H), 8.32 (2H), 7.96 (2H), 7.38 (2H), 6.70-6.56 (4H), 3.89 (6H), 3.86 (6H); HRMS (ESI): m/z calcd. for C₃₀H₂₅O₆⁺ [M+H]⁺, 481.1646; found: 481.1643.

(15) Preparation of Monomer M15

1 g of 2,6-dibromonaphthalene (CAS: 13720-064) was completely dissolved in an aqueous solution of 1,4-dioxane, and then 1.3 g of 2,4-dimethoxyphenylboronic acid, 0.4 g of tetrakis(triphenylphosphine)palladium and 3.4 g of Na₂CO₃ were added. The mixed system was heated to 110° C. and refluxed overnight. After the reaction was completed, the reaction mixture was cooled to room temperature and concentrated by rotary evaporation to remove the solvent. The residue was dissolved in dichloromethane, and the resulting solution was washed with water three times. The organic layer was dried over anhydrous Na₂SO₄ and concentrated again by rotary evaporation. The residue was prepared for column chromatography to isolate a product M15.

¹H NMR (500 MHz, CDCl₃) δ 7.93 (2H), 7.86 (2H), 7.66 (2H), 7.37 (2H), 6.64-6.61 (4H), 3.89 (6H), 3.83 (6H); HRMS (ESI): m/z calcd. for C₂₆H₂₄O₄⁺ [M+H]⁺, 401.1751; found: 401.1748.

(16) Preparation of Monomer M16

1,3-Dibromobenzene (CAS: 108-36-1) was completely dissolved in an aqueous solution of 1,4-dioxane (dioxane:water=5:1), and then 3.6 g of 2,4-dimethoxyphenylboronic acid, 0.4 g of tetrakis(triphenylphosphine)palladium and 4.2 g of sodium carbonate were added. The mixed system was heated to 100° C. and refluxed overnight. After the reaction was completed, the reaction mixture was cooled to room temperature and concentrated by rotary evaporation to remove the solvent. The residue was dissolved in dichloromethane, and the resulting solution was washed with water three times. The organic layer was dried over anhydrous Na₂SO₄ and concentrated again by rotary evaporation. The residue was prepared for column chromatography to isolate a monomer M16.

¹H NMR (400 MHz, CDCl₃) δ 7.68-7.62 (1H), 7.52-7.39 (3H), 7.31 (2H), 6.65-6.51 (4H), 3.87 (6H), 3.82 (6H); HRMS (MALDI-TOF): m/z calcd. for C₂₂H₂₃O₄⁺ [M+H]⁺, 351.1591; found: 351.1599.

(17) Preparation of Monomer M17

1.5 g of 2,7-dibromotetraphenylethylene (CAS: 859315-37-0) was completely dissolved in an aqueous solution of 1,4-dioxane, and then 1.4 g of 2,4-dimethoxyphenylboronic acid, 0.4 g of tetrakis(triphenylphosphine)palladium and 3.1 g of Na₂CO₃ were added. The mixed system was heated to 110° C. and refluxed overnight. After the reaction was completed, the reaction mixture was cooled to room temperature and concentrated by rotary evaporation to remove the solvent. The residue was dissolved in dichloromethane, and the resulting solution was washed with water three times. The organic layer was dried over anhydrous Na₂SO₄ and concentrated again by rotary evaporation. The residue was prepared for column chromatography to isolate a monomer M17.

¹H NMR (500 MHz, CDCl₃) δ 7.33-7.28 (4H), 7.27-7.23 (2H), 7.19-7.08 (14H), 6.56 (4H), 3.86 (6H), 3.79 (6H); HRMS (ESI): m/z calcd. for C₄₂H₃₆O₄⁺ [M+H]⁺, 605.2686; found: 605.2697.

(18) Preparation of Monomer M18

1 g of 2,7-dibromonaphthalene (CAS: 58556-75-5) was completely dissolved in an aqueous solution of 1,4-dioxane, and then 1.4 g of 2,4-dimethoxyphenylboronic acid, 0.4 g of tetrakis(triphenylphosphine)palladium and 3.4 g of Na₂CO₃ were added. The mixed system was heated to 110° C. and refluxed overnight. After the reaction was completed, the reaction mixture was cooled to room temperature and concentrated by rotary evaporation to remove the solvent. The residue was dissolved in dichloromethane, and the resulting solution was washed with water three times. The organic layer was dried over anhydrous Na₂SO₄ and concentrated again by rotary evaporation. The residue was prepared for column chromatography to isolate a monomer M18.

¹H NMR (500 MHz, CDCl₁) δ 7.94 (2H), 7.85 (2H), 7.65 (2H), 7.39-7.36 (2H), 6.62 (4H), 3.88 (6H), 3.83 (6H); HRMS (ESI): m/z calcd. for C₂₆H₂₄O₄⁺ [M+H]⁺, 401.1750; found: 401.1747.

(19) Preparation of Monomer M19

o-4,4′-dibromoterphenyl (CAS: 24253-43-8) was completely dissolved in an aqueous solution of 1,4-dioxane (dioxane:water=5:1), and then 3.6 g of 2,4-dimethoxyphenylboronic acid, 0.4 g of tetrakis(triphenylphosphine)palladium and 4.2 g of sodium carbonate were added. The mixed system was heated to 100° C. and refluxed overnight. After the reaction was completed, the reaction mixture was cooled to room temperature and concentrated by rotary evaporation to remove the solvent. The residue was dissolved in dichloromethane, and the resulting solution was washed with water three times. The organic layer was dried over anhydrous Na₂SO₄ and concentrated again by rotary evaporation. The residue was prepared for column chromatography to isolate a monomer M19.

¹H NMR (500 MHz, CDCl₃) δ 7.50-7.48 (2H), 7.43-7.41 (2H), 7.39 (4H), 7.25 (2H), 7.22 (2H), 6.69-6.38 (4H), 3.84 (6H), 3.78 (6H); HRMS (ESI): m/z calcd. for C₃₄H₃₀O₄Na⁺ [M+Na]⁺, 525.2036; found: 525.2039.

(20) Preparation of Monomer M20

To a 100 mL flask were added 0.6 g of 4,4′-dibromodione (CAS: 33170-68-2), 0.7 g of 2,4-dimethoxyphenylboronic acid, 0.1 g of tetrakis(triphenylphosphine)palladium and 0.5 g of sodium carbonate, followed by 6 mL of water and 30 mL of dioxane (water:dioxane=1:5). The mixture was stirred at reflux overnight. After the reaction was completed, the mixture was cooled to room temperature and subjected to rotary evaporation to remove the solvent. A solution of sodium carbonate in water was added, and dichloromethane was added for extraction three times. The organic solvents were pooled, and the organic layer was washed with saturated brine, dried over anhydrous sodium sulfate, and concentrated by rotary evaporation to remove the solvent. The residue was subjected to column chromatography to isolate a monomer M20 in the form of a bright yellowish green solid.

¹H NMR (500 MHz, CDCl₃) δ 8.03 (4H), 7.65 (4H), 7.31 (2H), 6.93 (1H), 6.69-6.55 (4H), 3.87 (6H), 3.83 (6H); HRMS (ESI): m/z calcd. for C₃₀H₂₉O₆⁺ [M+H]⁺, 497.1959; found: 497.1962.

(21) Preparation of Monomer M21

In a 100 mL three-necked flask, 1.2 g of 4,4′-dibromodione (CAS: 33170-68-2) was dissolved in 25 mL of dichloromethane, and 0.5 mL of triethylamine was added, followed by 1.4 mL of boron trifluoride diethyl ether coordination complex under nitrogen atmosphere. The mixture was stirred at room temperature overnight, quenched with saturated aqueous sodium bicarbonate solution, and subjected to liquid separation. The solvent was removed by rotary evaporation, and acetone/n-hexane was added for recrystallization to obtain a yellow solid. Thereafter, 0.7 g of 2,4-dimethoxyphenylboronic acid, 0.15 g of tetrakis(triphenylphosphine)palladium and 0.530 g of sodium carbonate were added to the flask, followed by 6 mL of water and 30 mL of dioxane. The mixture was stirred at reflux overnight. After the reaction was completed, the mixture was cooled to room temperature and subjected to rotary evaporation to remove the solvent. A solution of sodium carbonate in water was added, and dichloromethane was added for extraction three times. The organic solvents were pooled, and the organic layer was washed with saturated brine, dried over anhydrous sodium sulfate, and concentrated by rotary evaporation to remove the solvent. The residue was subjected to column chromatography to isolate a monomer M21 in the form of a bright yellowish green solid.

¹H NMR (500 MHz, CDCl₃) δ 8.15 (4H), 7.70 (4H), 7.31 (2H), 7.23 (1H), 6.65-6.55 (4H), 3.87 (6H), 3.83 (6H); HRMS (ESI): m/z calcd. for C₃₁H₂₇B₁F₂O₆⁺ [M+H]⁺, 545.1947; found: 545.1944.

(22) Preparation of Monomer M22

To a 500 mL round-bottomed flask were sequentially added 3.6 g of 1-bromo-4′-formaldehyde (CAS: 1122-91-4), 2.0 g of pyrrole, 1.6 g of benzaldehyde and 2.1 g of salicylic acid, and finally 250 mL of o-xylene as a solvent. The mixture was stirred and heated at reflux under nitrogen atmosphere for 6 h. Then 3 g of 2,4-dimethoxyphenylboronic acid and 0.4 g of tetrakis(triphenylphosphine)palladium catalyst were added. The mixture was allowed to react overnight. The mixture was cooled to room temperature and distilled under reduced pressure. A dichloromethane methanol solution was added for recrystallization. The mixture was subjected to suction filtration, and the solid was collected. The solid was subjected to column chromatography (neutral alumina) (eluent: dichloromethane:petroleum ether=1:2) to obtain a monomer M22 in the form of a purple powder.

¹H NMR (500 MHz, CDCl₃) δ 9.03-9.01 (4H), 8.88-8.86 (4H), 8.27-8.24 (8H), 7.94-7.92 (4H), 7.79-7.75 (6H), 7.62-7.60 (2H), 6.73-6.72 (4H), 2.68 (2H); HRMS (ESI): m/z calcd. for C₆₀H₄₆O₃ [M+H]⁺, 887.3592; found: 887.3588.

[2] Preparation of Monomer of Cage-Like Molecules with Methoxy Side Chain

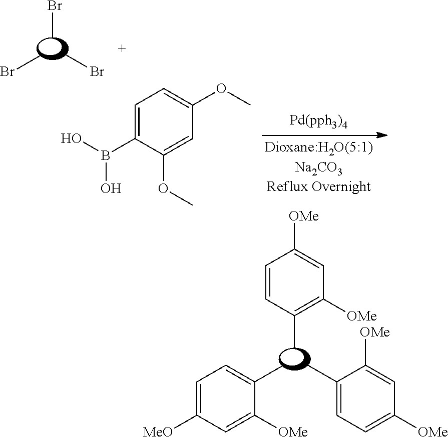

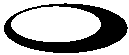

| | |
|---|---|
| 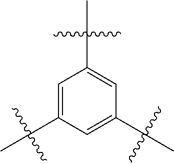 [SMILES: *c1cc(*)cc(*)c1] | M23 |
| 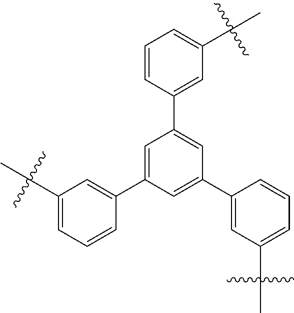 [SMILES: *c1cccc(-c2cc(-c3cccc(*)c3)cc(-c3cccc(*)c3)c2)c1] | M24 |

(1) Preparation of Monomer M23

A monomer M23 was synthesized according to the method described in Suzuki, Akira; Akita, Munetaka; Yoshizawa, Michito. Chemical Communications, 2016, 52, 65, 10024-10027.

(2) Preparation of Monomer M24

1.8 g of 1,3,5-tris(3-bromophenyl)benzene (CAS: 96761-85-2) was completely dissolved in an aqueous solution of 1,4-dioxane (dioxane:water=5:1), and then 2.4 g of 2,4-dimethoxyphenylboronic acid, 0.4 g of tetrakis(triphenylphosphine)palladium and 2.1 g of sodium carbonate were added. The mixed system was heated to 100° C. and refluxed overnight. After the reaction was completed, the reaction mixture was cooled to room temperature and concentrated by rotary evaporation to remove the solvent. The residue was dissolved in dichloromethane, and the resulting solution was washed with water three times. The organic layer was dried over anhydrous Na₂SO₄ and concentrated again by rotary evaporation. The residue was prepared for column chromatography to isolate a monomer M24.

¹H NMR (500 MHz, CDCl₃) δ 7.86 (3H), 7.82 (3H), 7.63 (3H), 7.56-7.43 (6H), 7.32 (3H), 6.59 (6H), 3.86 (9H), 3.81 (9H).

[3] Preparation of a Macrocyclic Monomer with Dibutoxyl and 2-(5-bromopentyloxy)-4-methoxy Side Chain

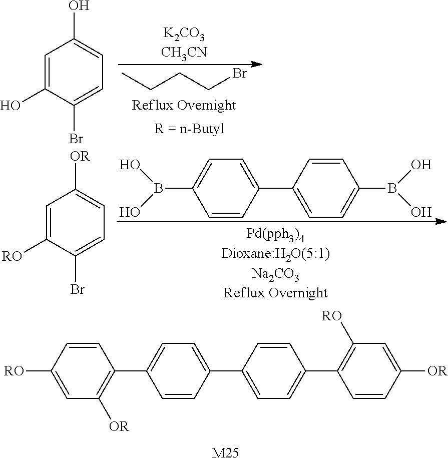

(1) Preparation of a Quaterphenyl Macrocyclic Monomer with Butyl Side Chain

2.17 g of n-butyl bromide was added to a 250 mL three-necked flask and heated at reflux. Then 1 g of 4-bromo-1,3-resorcinol (CAS: 6626-15-9) was dissolved in 40 mL of acetonitrile, and the resulting solution was added dropwise to the reaction system. The reaction system was allowed to react overnight. After the reaction was completed, the heating was stopped, and the mixture was filtered to remove potassium carbonate. The reaction solution was concentrated by rotary evaporation, and the residue was subjected to column chromatography to isolate 4-bromo-1,3-dibutoxybenzene reaction product.

¹H NMR (400 MHz, CDCl₃) δ 7.38 (1H), 6.47 (1H), 6.37 (1H), 3.96 (4H), 1.99-1.64 (4H), 1.51 (4H), 0.98 (6H).

1.2 g of 4-bromo-1,3-dibutoxybenzene was completely dissolved in an aqueous solution of 1,4-dioxane (dioxane:water=5:1), and then 0.5 g of 4,4′-biphenyldiboronic acid, 0.4 g of tetrakis(triphenylphosphine)palladium and 1.6 g of sodium carbonate were added. The mixed system was heated to 100° C. and refluxed overnight. After the reaction was completed, the reaction mixture was cooled to room temperature and concentrated by rotary evaporation to remove the solvent. The residue was dissolved in dichloromethane, and the resulting solution was washed with water three times. The organic layer was dried over anhydrous Na₂SO₄ and concentrated again by rotary evaporation. The residue was prepared for column chromatography to isolate a monomer M25.

¹H NMR (400 MHz, CDCl₃) δ 7.67 (4H), 7.61 (4H), 7.30 (2H), 6.57 (4H), 3.99 (8H), 1.92-1.66 (8H), 1.61-1.38 (8H), 1.00 (6H), 0.94 (6H).

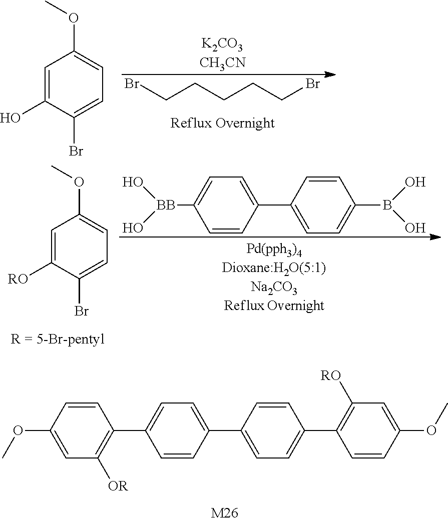

(2) Preparation of a Macrocyclic Monomer with 2-(5-bromopentyloxy)-4-methoxy Side Chain

2.4 g of 1,5-dibromopentane was added to a 250 mL three-necked flask and heated at reflux. Then 1 g of 2-bromo-5-methoxyphenol (CAS: 63604-94-4) was dissolved in 40 mL of acetonitrile, and the resulting solution was added dropwise to the reaction system. The reaction system was allowed to react overnight. After the reaction was completed, the heating was stopped, and the mixture was filtered to remove potassium carbonate. The reaction solution was concentrated by rotary evaporation, and the residue was subjected to column chromatography to isolate 4-methoxy-2-(5-bromo-n-pentyloxy)bromobenzene reaction product.

¹H NMR (400 MHz, CDCl₃) δ 7.40 (1H), 6.46 (1H), 6.39 (1H), 4.00 (2H), 3.79 (3H), 3.46 (2H), 2.05-1.91 (2H), 1.92-1.82 (2H), 1.68 (2H).

1.5 g of 4-methoxy-2-(5-bromo-n-pentyloxy)bromobenzene was completely dissolved in an aqueous solution of 1,4-dioxane (dioxane:water=5:1), and then 0.5 g of 4,4′-biphenyldiboronic acid, 0.4 g of tetrakis(triphenylphosphine)palladium and 1.6 g of sodium carbonate were added. The mixed system was heated to 100° C. and refluxed overnight. After the reaction was completed, the reaction mixture was cooled to room temperature and concentrated by rotary evaporation to remove the solvent. The residue was dissolved in dichloromethane, and the resulting solution was washed with water three times. The organic layer was dried over anhydrous Na₂SO₄ and concentrated again by rotary evaporation. The residue was prepared for column chromatography to isolate a monomer M26.

¹H NMR (400 MHz, CDCl₃) δ 7.60 (8H), 7.25 (2H), 6.60-6.38 (4H), 3.92 (4H), 3.79 (6H), 3.31 (4H), 1.80 (4H), 1.72 (4H), 1.58-1.49 (4H); HRMS (ESI): m/z calcd. for C₃₆H₄₀Br₂O₄ [M+Na]⁺: 719.1168; found: 719.1162.

[4] Preparation of a Hexamethoxybiphenyl Macrocyclic Monomer

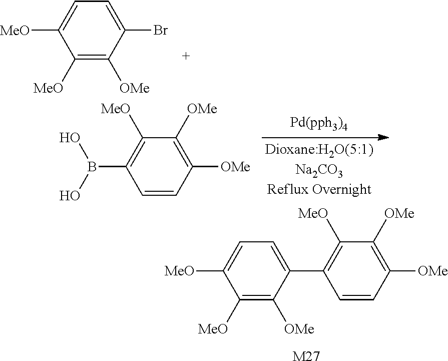

A monomer M27 was synthesized with reference to the method described in Mulla, Shafeek A. R.; Chavan, Santosh S.; Pathan, Mohsinkhan Y.; Inamdar, Suleman M.; Shaikh, Taufeckaslam M. Y.; RSC Advances, 2015, 5, 31, 24675-24680.

II. A synthesis method for the macrocyclic and cage-like molecule based on biphen[n]arene of step 2 is as follows:

[1] Synthesis of Macrocyclic Compounds with Trimer or Higher Degree of Polymerization from Linear Molecules

The synthesis route is as follows:

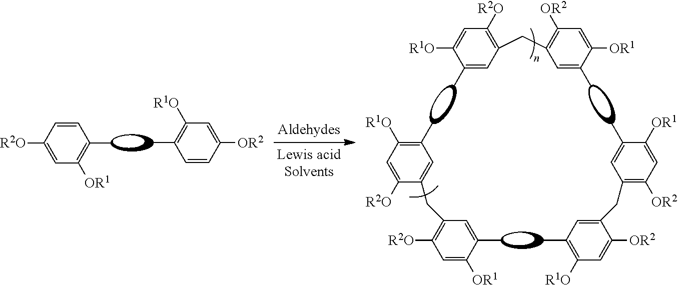

| 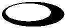 | Reactant | Solvent | Lewis acid | Degree of polymer- ization | Side chain | No. |
|---|---|---|---|---|---|---|
| 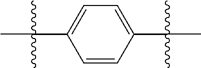 [SMILES: *c1ccc(*)cc1] 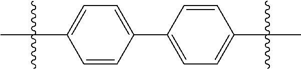 [SMILES: *c1ccc(-c2ccc(*)cc2)cc1] 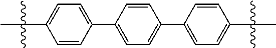 [SMILES: *c1ccc(-c2ccc(-c3ccc(*)cc3)cc2)cc1] 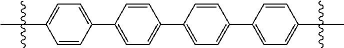 [SMILES: *c1ccc(-c2ccc(-c3ccc(-c4ccc(*)cc4)cc3)cc2)cc1] 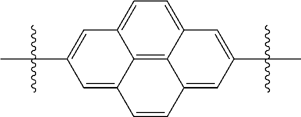 [SMILES: *c1cc2ccc3cc(*)cc4ccc(c1)c2c34] | Paraform- aldehyde Paraform- aldehyde Paraform- aldehyde Paraform- aldehyde Paraform- aldehyde | Trichloro/ bromomethane Tetrachloromethane Dichloro/ bromoethane Trichloro/ bromoethane Tetrachloro/ bromoethane Monochloro/ bromopropane Monochloro bromobutane Monochloro/ bromopentane Monochloro/ bromohexane Monochloro/ bromoheptane Monochloro/ bromooctane Monochloro/ bromononane Monochloro/ bromodecane (The solvents above are all reactive) | p-Toluenesulfonic acid Triflic acid Boron trifluoride diethyl ether Ferric chloride Aluminium trichloride (The Lewis acids above can all catalyze a reaction) | 3, 5 3 3-6 3 3, 4 | R¹ = R² = Me R¹ = R² = Me R¹ = R² = Me R¹ = R² = Me R¹ = R² = Me | 1 2 3 4 5 |
| 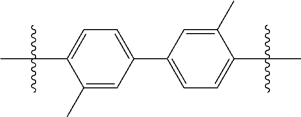 [SMILES: *c1ccc(-c2ccc(*)c(C)c2)cc1C] | Paraform- aldehyde | | | 3-5 | R¹ = R² = Me | 6 |
| 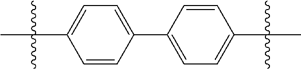 [SMILES: *c1ccc(-c2ccc(*)cc2)cc1] | Paraform- aldehyde Paraform- aldehyde | | | 3 3, 4 | R¹ = R² = n-Butyl R₁ = 5-Br- pentyl | 7 8 |
| | | | | | R₂ = Me | |
| 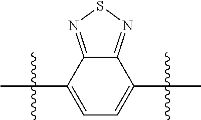 [SMILES: *c1ccc(*)c2nsnc12] | Paraform- aldehyde | | | 3 | R¹ = R² = Me | 9 |
| 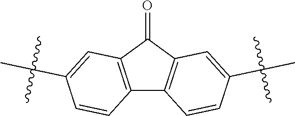 [SMILES: *c1ccc2c(c1)C(=O)c1cc(*)ccc1-2] | Paraform- aldehyde | | | 2-5 | R¹ = R² = Me | 10 |
| 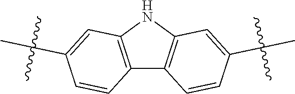 [SMILES: *c1ccc2c(c1)[nH]c1cc(*)ccc12] | Paraform- aldehyde | | | 3 | R¹ = R² = Me | 11 |
| 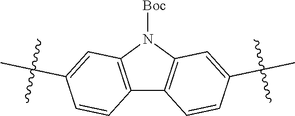 [SMILES: *c1ccc2c3ccc(*)cc3n(C(=O)OC(C)(C)C)c2c1] | Paraform- aldehyde | | | 3 | R¹ = R² = Me | 12 |
| 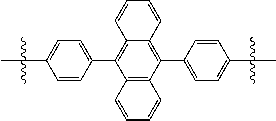 [SMILES: *c1ccc(-c2c3ccccc3c(-c3ccc(*)cc3)c3ccccc23)cc1] | Paraform- aldehyde | | | 3 | R¹ = R² = Me | 13 |
| 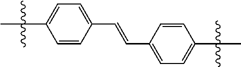 [SMILES: *c1ccc(/C=C/c2ccc(*)cc2)cc1] | Paraform- aldehyde | | | 3 | R¹ = R² = Me | 14 |
| 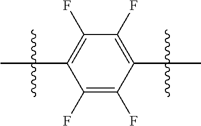 [SMILES: *c1c(F)c(F)c(*)c(F)c1F] | Paraform- aldehyde | | | 3 | R¹ = R² = Me | 15 |
| 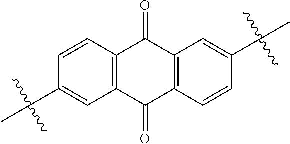 [SMILES: *c1ccc2c(c1)C(=O)c1ccc(*)cc1C2=O] | Paraform- aldehyde | | | 3 | R¹ = R² = Me | 16 |
| 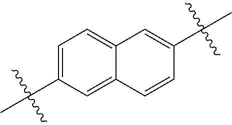 [SMILES: *c1ccc2cc(*)ccc2c1] | Paraform- aldehyde | | | 3-5 | R¹ = R² = Me | 17 |

The degree of polymerization in the above table refers to the number of repeat units contained in a supramolecular macrocyclic compound. For example: a macrocycle with a degree of polymerization of 3 is a macrocyclic structure consisting of 3 repeat units bridged by methylene.

(1) Preparation of Macrocyclic Arene 1

To a 500 mL round-bottomed flask were added 1 g of monomer M1 and 0.1 g of paraformaldehyde, and 250 mL of a haloalkane (dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, or monobromodecane) was poured into the flask as a solvent for dissolution. 1.5 equivalents of a Lewis acid catalyst (p-toluenesulfonic acid or triflic acid or boron trifluoride diethyl ether or ferric trichloride or aluminum trichloride) was added with stirring. The reaction was monitored by thin-layer chromatography. After 30 min, the reaction was completed. The reaction mixture was quenched with 50 mL of a saturated solution of sodium bicarbonate, washed with 50 mL of a saturated solution of sodium chloride, and dried over anhydrous sodium sulfate. The resulting mixture was subjected to silica gel column chromatography to isolate macrocyclic arene 1 trimeric and pentameric ring formation products.

Trimer: ¹H NMR (400 MHz, CDCl₃) δ 7.39 (12H), 6.92 (6H), 6.54 (6H), 3.89 (6H), 3.86 (18H), 3.80 (18H); HRMS (MALDI-TOF): m/z calcd. for C₆₉H₆₇O₁₂⁺ [M]⁺, 1087.4627; found: 1087.4630.

Monocrystal data: Triclinic, space group P-1, a=11.4902(14), b=18.050(2), c=21.034(3) Å, α=67, β=85, γ=87°, V=4003.7(9) Å³, Z=4, T=203 K, μ(Mo/Kα)=0.71073 Å, ρ_{calc}=1.066 g/cm³, theta range for data collection 1.053 to 26.999°, index ranges −13≤h≤14, −23≤k≤23, −17≤l≤26, reflections collected 17477, data completeness 0.982, goodness-of-fit 1.026. The final R₁ was 0.0889 (I>2σ(I)) and wR₂ was 0.3407 (all data).

Pentamer: ¹H NMR (400 MHz, CDCl₃) δ 7.43 (20H), 7.07 (10H), 6.51 (10H), 3.89 (10H), 3.85 (30H), 3.76 (30H); HRMS (MALDI-TOF): m/z calcd. for C₁₁₅H₁₁₀O₂₀Na⁺ [M+Na]⁺, 1834.7516; found: 1834.7520.

(2) Preparation of Macrocyclic Arene 2

To a 500 mL round-bottomed flask were added 1 g of monomer M2 and 0.1 g of paraformaldehyde, and 300 mL of a haloalkane (dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, or monobromodecane) was poured into the flask as a solvent for dissolution. 1.5 equivalents of a Lewis acid catalyst (p-toluenesulfonic acid or triflic acid or boron trifluoride diethyl ether or ferric trichloride or aluminum trichloride) was added with stirring. The reaction was monitored by thin-layer chromatography. After 30 min, the reaction was completed. The reaction mixture was quenched with 50 mL of a saturated solution of sodium bicarbonate, washed with 50 mL of a saturated solution of sodium chloride, and dried over anhydrous sodium sulfate. The resulting mixture was subjected to silica gel column chromatography to isolate a macrocyclic arene 2 trimeric ring formation product.

Trimer: ¹H NMR (500 MHz, CDCl₃) δ 7.63 (12H), 7.55 (12H), 7.04 (6H), 6.60 (6H), 3.97 (6H), 3.91 (18H), 3.86 (18H); HRMS (MALDI-TOF): m/z calcd. for C₈₇H₇₈O₁₂⁺ [M]⁺, 1314.5493; found: 1314.5490 [M]⁺, 1337.5388 [M+Na]⁺.

(3) Preparation of Macrocyclic Arene 3

To a 500 mL round-bottomed flask were added 1 g of monomer M3 and 0.5 g of paraformaldehyde, and 300 mL of a haloalkane (dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, or monobromodecane) was poured into the flask as a solvent for dissolution. 3 equivalents of a Lewis acid catalyst (p-toluenesulfonic acid or triflic acid or boron trifluoride diethyl ether or ferric trichloride or aluminum trichloride) was added with stirring. The reaction was monitored by thin-layer chromatography. After 20 min, the reaction was completed. The reaction mixture was quenched with 100 mL of a saturated solution of sodium bicarbonate, washed with 50 mL of a saturated solution of sodium chloride, and dried over anhydrous sodium sulfate. The resulting mixture was subjected to silica gel column chromatography to isolate macrocyclic arene 3 trimeric, tetrameric, pentameric and hexameric ring formation products.

Trimer: ¹H NMR (400 MHz, CDCl₃) δ 7.67 (12H), 7.63 (12H), 7.55 (12H), 7.02 (6H), 6.59 (6H), 3.95 (6H), 3.89 (18H), 3.85 (18H); HRMS (ESI): m/z calcd. for C₁₀₅H₉₀NaO₁₂⁺ [M+Na]⁺, 1566.6358; found: 1566.6354.

Tetramer: ¹H NMR (500 MHz, CDCl₃) δ 7.67 (16H), 7.62 (16H), 7.54 (16H), 6.99 (8H), 6.60 (8H), 3.95 (8H), 3.91 (24H), 3.85 (24H); HRMS (ESI): m/z calcd. for C₁₄₀H₁₂₀NaO₁₆⁺ [M+Na]⁺, 2080.8502; found: 2080.8510.

Pentamer: ¹H NMR (400 MHz, CDCl₃) δ 7.68 (20H), 7.64 (20H), 7.55 (20H), 7.11 (10H), 6.58 (10H), 3.94 (10H), 3.91 (30H), 3.84 (30H).

Hexamer: ¹H NMR (400 MHz, CDCl₃) δ 7.68 (24H), 7.64 (24H), 7.55 (24H), 7.11 (12H), 6.57 (12H), 3.94 (12H), 3.90 (36H), 3.83 (36H).

(4) Preparation of Macrocyclic Arene 4

To a 500 mL round-bottomed flask were added 0.3 g of monomer M4 and 0.3 g of paraformaldehyde, and 200 mL of a haloalkane (dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, or monobromodecane) was poured into the flask as a solvent for dissolution. 5 equivalents of a Lewis acid catalyst (p-toluenesulfonic acid or triflic acid or boron trifluoride diethyl ether or ferric trichloride or aluminum trichloride) was added with stirring. The reaction was monitored by thin-layer chromatography. After 60 min, the reaction was completed. The reaction mixture was quenched with 100 mL of a saturated solution of sodium bicarbonate, washed with 50 mL of a saturated solution of sodium chloride, and dried over anhydrous sodium sulfate. The resulting mixture was subjected to silica gel column chromatography to isolate a macrocyclic arene 4 trimeric ring formation product.

Trimer: ¹H NMR (400 MHz, CDCl₃) δ 7.70 (24H), 7.64 (12H), 7.56 (12H), 7.02 (6H), 6.60 (6H), 3.95 (6H), 3.90 (18H), 3.86 (18H).

(5) Preparation of Macrocyclic Arene 5

To a 500 mL round-bottomed flask were added 1 g of monomer M5 and 0.1 g of paraformaldehyde, and 200 mL of a haloalkane (dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, or monobromodecane) was poured into the flask as a solvent for dissolution. 1.5-fold equivalents of a Lewis acid catalyst (p-toluenesulfonic acid or triflic acid or boron trifluoride diethyl ether or ferric trichloride or aluminum trichloride) was added with stirring. The reaction was monitored by thin-layer chromatography. After 30 min, the reaction was completed. The reaction mixture was quenched with 50 mL of a saturated solution of sodium bicarbonate, washed with 50 mL of a saturated solution of sodium chloride, and dried over anhydrous sodium sulfate. The resulting mixture was subjected to silica gel column chromatography to isolate macrocyclic arene 5 trimeric and tetrameric ring formation products.

Trimer: ¹H NMR (500 MHz, CDCl₃) δ 8.24 (12H), 8.01 (12H), 7.25 (6H), 6.66 (6H), 4.08 (6H), 3.95 (18H), 3.84 (18H); HRMS (ESI): m/z calcd. for C₉₉H₇₉O₁₂⁺ [M]⁺, 1460.5600; found: 1460.5604.

Tetramer: ¹H NMR (500 MHz, CDCl₃) δ 8.22 (16H), 7.97 (16H), 7.23 (8H), 6.66 (8H), 4.06 (8H), 3.96 (24H), 3.83 (24H); HRMS (MALDI-TOF): m/z calcd. for C₁₃₂H₁₀₄NaO₁₆⁺ [M+Na]⁺, 1968.7250; found: 1968.7220.

(6) Preparation of Macrocyclic Arene 6

To a 500 mL round-bottomed flask were added 1 g of monomer M6 and 0.1 g of paraformaldehyde, and 200 mL of a haloalkane (dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, or monobromodecane) was poured into the flask as a solvent for dissolution. 1-fold equivalent of a Lewis acid catalyst (p-toluenesulfonic acid or triflic acid or boron trifluoride diethyl ether or ferric trichloride or aluminum trichloride) was added with stirring. The reaction was monitored by thin-layer chromatography. After 30 min, the reaction was completed. The reaction mixture was quenched with 100 mL of a saturated solution of sodium bicarbonate, washed with 50 mL of a saturated solution of sodium chloride, and dried over anhydrous sodium sulfate. The resulting mixture was subjected to silica gel column chromatography to isolate macrocyclic arene 6 trimeric, tetrameric and pentameric ring formation products.

Trimer: ¹H NMR (400 MHz, CDCl₃) δ 7.49 (6H), 7.44 (6H), 7.17 (6H), 6.78 (6H), 6.55 (6H), 3.93 (6H), 3.89 (18H), 3.78 (18H), 2.18 (18H); HRMS (MALDI-TOF): m/z calcd. for C₉₃H₉₀O₁₂⁺ [M]⁺, 1399.6466; found: 1399.6462.

Tetramer: ¹H NMR (400 MHz, CDCl₃) δ 7.46 (8H), 7.41 (8H), 7.18 (8H), 6.88 (8H), 6.53 (8H), 3.91 (8H), 3.88 (24H), 3.76 (24H), 3.76 (24H), 2.17 (24H); HRMS (MALDI-TOF): m/z calcd. for C₁₂₄H₁₂₀O₁₆⁺ [M]⁺, 1865.8610; found: 1865.8605.

Pentamer: ¹H NMR (400 MHz, CDCl₃) δ 7.45 (10H), 7.41 (10H), 7.17 (10H), 6.75 (10H), 6.55 (10H), 3.91 (10H), 3.88 (30H), 3.77 (30H), 2.15 (30H); HRMS (MALDI-TOF): m/z calcd. for C₁₅₅H₁₅₀O₂₀⁺ [M+Na]⁺, 2332.0754; found: 2355.0650.

(7) Preparation of Macrocyclic Arene 7

To a 500 mL round-bottomed flask were added 1 g of monomer M25 and 0.1 g of paraformaldehyde, and 300 mL of a haloalkane (dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, or monobromodecane) was poured into the flask as a solvent for dissolution. 1.2 equivalents of a Lewis acid catalyst (p-toluenesulfonic acid or triflic acid or boron trifluoride diethyl ether or ferric trichloride or aluminum trichloride) was added with stirring. The reaction was monitored by thin-layer chromatography. After 30 min, the reaction was completed. The reaction mixture was quenched with 50 mL of a saturated solution of sodium bicarbonate, washed with 50 mL of a saturated solution of sodium chloride, and dried over anhydrous sodium sulfate. The resulting mixture was subjected to silica gel column chromatography to isolate a macrocyclic arene 7 trimeric ring formation product.

Trimer: ¹H NMR (400 MHz, CDCl₃) δ 7.60 (12H), 7.53 (12H), 7.02 (6H), 6.54 (6H), 4.01 (6H), 3.98 (12H), 3.94(12H), 1.73 (24H), 1.45(24H), 0.92 (36H); HRMS (MALDI-TOF): m/z calcd. for C₁₂₃H₁₅₀O₁₂⁺ [M]⁺, 1820.1161; found, 1820.1160.

(8) Preparation of Macrocyclic Arene 8

To a 500 mL round-bottomed flask were added 1 g of monomer M26 and 0.1 g of paraformaldehyde, and 200 mL of a haloalkane (dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, or monobromodecane) was poured into the flask as a solvent. 1 equivalent of a Lewis acid catalyst (p-toluenesulfonic acid or triflic acid or boron trifluoride diethyl ether or ferric trichloride or aluminum trichloride) was added with stirring. The reaction was monitored by thin-layer chromatography. After 30 min, the reaction was completed. The reaction mixture was quenched with 50 mL of a saturated solution of sodium bicarbonate, washed with 50 mL of a saturated solution of sodium chloride, and dried over anhydrous sodium sulfate. The resulting mixture was subjected to silica gel column chromatography to isolate macrocyclic arene 8 trimeric and tetrameric ring formation products.

Trimer: ¹H NMR (400 MHz, CDCl₃) δ 7.61 (12H), 7.52 (12H), 7.01 (6H), 6.56 (6H), 3.97 (12H), 3.93 (6H), 3.86 (18H), 3.35 (12H), 1.84 (12H), 1.74 (12H), 1.58 (12H); HRMS (MALDI-TOF): m/z calcd. for C₁₁₁H₁₂₀Br₆O₁₂⁺ [M]⁺, 2125.3852; found: 2125.3828.

Tetramer: ¹H NMR (400 MHz, CDCl₃) δ 7.59 (16H), 7.51 (16H), 6.97 (8H), 6.56 (8H), 3.97 (16H), 3.92 (8H), 3.88 (24H), 3.34 (16H), 1.82 (16H), 1.74 (16H), 1.58 (16H).

(9) Preparation of Macrocyclic Arene 9

To a 500 mL round-bottomed flask were added 1 g of monomer M7 and 0.2 g of paraformaldehyde, and 200 mL of a haloalkane (dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, or monobromodecane) was poured into the flask as a solvent for dissolution. 1 equivalent of a Lewis acid catalyst (p-toluenesulfonic acid or triflic acid or boron trifluoride diethyl ether or ferric trichloride or aluminum trichloride) was added with stirring. The reaction was monitored by thin-layer chromatography. After 20 min, the reaction was completed. The reaction mixture was quenched with 50 mL of a saturated solution of sodium bicarbonate, washed with 50 mL of a saturated solution of sodium chloride, and dried over anhydrous sodium sulfate. The resulting mixture was subjected to silica gel column chromatography to isolate a macrocyclic arene 9 trimeric ring formation product.

Trimer: ¹H NMR (500 MHz, Methylene Chloride-d₂) δ 8.16-7.56 (6H), 7.41 (6H), 6.83 (6H), 4.19 (6H), 4.10 (18H), 3.93 (18H); HRMS (ESI): m/z calcd for C₆₉H₆₀N₆O₁₂S₃⁺ [M]⁺, 2125.3852; found: 2125.3828.

(10) Preparation of Macrocyclic Arene 10

To a 500 mL round-bottomed flask were added 1 g of monomer M8 and 0.2 g of paraformaldehyde, and 200 mL of a haloalkane (dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, or monobromodecane) was poured into the flask as a solvent for dissolution. 2 equivalents of a Lewis acid catalyst (p-toluenesulfonic acid or triflic acid or boron trifluoride diethyl ether or ferric trichloride or aluminum trichloride) was added with stirring. The reaction was monitored by thin-layer chromatography. After 20 min, the reaction was completed. The reaction mixture was quenched with 100 mL of a saturated solution of sodium bicarbonate, washed with 50 mL of a saturated solution of sodium chloride, and dried over anhydrous sodium sulfate. The resulting mixture was subjected to silica gel column chromatography to isolate macrocyclic arene 10 dimeric, trimeric, tetrameric and pentameric ring formation products.

Dimer: ¹H NMR (500 MHz, CDCl₃) δ 7.72 (4H), 7.50 (4H), 7.46-7.42 (4H), 7.28 (4H), 6.56 (4H), 3.96 (4H), 3.91 (12H), 3.84 (12H); HRMS (ESI): m/z calcd. for C₆₀H₄₉O₁₉⁺ [M+H]⁺, 929.3320; found: 929.3316.

Trimer: ¹H NMR (500 MHz, CDCl₃) δ 7.77 (6H), 7.53-7.41 (12H), 6.95 (6H), 6.57 (6H), 3.92(6H), 3.89 (18H), 3.84 (18H); HRMS (ESI): m/z calcd. for C₉₀H₇₆NO₁₅⁺ [M+NH₄]⁺, 1411.5209; found: 1411.5208.

Tetramer: ¹H NMR (500 MHz, CDCl₃) δ 7.75 (8H), 7.50 (8H), 7.45 (8H), 6.94 (8H), 6.57 (8H), 3.91 (8H), 3.90 (24H), 3.83 (24H); HRMS (MALDI-TOF): m/z calcd. for C₁₂₀H₉₆O₂₀Na⁺ [M+Na]⁺, 1857.6529; found: 1880.6409.

Pentamer: ¹H NMR (500 MHz, CDCl₃) δ 7.76-7.71 (10H), 7.54 (10H), 7.46 (10H), 7.05 (10H), 6.55 (10H), 3.90 (40H), 3.81 (30H); HRMS (MALDI-TOF): m/z calcd. for C₁₅₀H₁₂₀ONa⁺ [M+Na]⁺, 2321.8152; found: 2344.8018.

(11) Preparation of Macrocyclic Arene 11

To a 500 mL round-bottomed flask were added 1 g of monomer M9 and 0.1 g of paraformaldehyde, and 200 mL of a haloalkane (dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, or monobromodecane) was poured into the flask as a solvent for dissolution. 0.5 equivalents of a Lewis acid catalyst (p-toluenesulfonic acid or triflic acid or boron trifluoride diethyl ether or ferric trichloride or aluminum trichloride) was added with stirring. The reaction was monitored by thin-layer chromatography. After 20 min, the reaction was completed. The reaction mixture was quenched with 50 mL of a saturated solution of sodium bicarbonate, washed with 50 mL of a saturated solution of sodium chloride, and dried over anhydrous sodium sulfate. The resulting mixture was subjected to silica gel column chromatography to isolate a macrocyclic arene 11 trimeric ring formation product.

Trimer: ¹H NMR (400 MHz, CDCl₃) δ 7.87-7.73 (9H), 7.34 (6H), 7.23 (6H), 7.06 (6H), 6.54 (6H), 4.00 (6H), 3.83 (18H), 3.75 (18H); HRMS (ESI): m/z calcd. for C₈₇H₇₆N₃O₁₂⁺ [M+H]⁺, 1354.5429; found: 1654.5400.

(12) Preparation of Macrocyclic Arene 12

To a 500 mL round-bottomed flask were added 1 g of monomer M10 and 50 mg of paraformaldehyde, and 200 mL of a haloalkane (dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, or monobromodecane) was poured into the flask as a solvent for dissolution. 0.5 equivalents of a Lewis acid catalyst (p-toluenesulfonic acid or triflic acid or boron trifluoride diethyl ether or ferric trichloride or aluminum trichloride) was added with stirring. The reaction was monitored by thin-layer chromatography. After 20 min, the reaction was completed. The reaction mixture was quenched with 100 mL of a saturated solution of sodium bicarbonate, washed with 50 mL of a saturated solution of sodium chloride, and dried over anhydrous sodium sulfate. The resulting mixture was subjected to silica gel column chromatography to isolate a macrocyclic arene 12 trimeric ring formation product.

Trimer: ¹H NMR (400 MHz, CDCl₃) δ 8.41 (6H), 7.88 (6H), 7.41 (6H), 7.07 (6H), 6.59 (6H), 3.96 (6H), 3.90 (18H), 3.82 (18H), 1.49 (27H); HRMS (ESI): m/z calcd. for C₁₀₂H₁₀₃N₄O₁₈⁺ [M+NH₄]⁺, 1672.7301; found: 1672.7295.

(13) Preparation of Macrocyclic Arene 13

To a 500 mL round-bottomed flask were added 0.5 g of monomer M11 and 0.3 g of paraformaldehyde, and 200 mL of a haloalkane (dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, or monobromodecane) was poured into the flask as a solvent for dissolution. 2 equivalents of a Lewis acid catalyst (p-toluenesulfonic acid or triflic acid or boron trifluoride diethyl ether or ferric trichloride or aluminum trichloride) was added with stirring. The reaction was monitored by thin-layer chromatography. After 2 h, the reaction was completed. The reaction mixture was quenched with 100 mL of a saturated solution of sodium bicarbonate, washed with 50 mL of a saturated solution of sodium chloride, and dried over anhydrous sodium sulfate. The resulting mixture was subjected to silica gel column chromatography to isolate a macrocyclic arene 13 trimeric ring formation product.

Trimer: ¹H NMR (400 MHz, CDCl₃) δ 7.80 (12H), 7.71 (12H), 7.44 (12H), 7.26 (12H), 7.22 (6H), 6.68 (6H), 4.04 (6H), 3.96 (18H), 3.95 (18H).

(14) Preparation of Macrocyclic Arene 14

To a 500 mL round-bottomed flask were added 1 g of monomer M12 and 0.2 g of paraformaldehyde, and 150 mL of a haloalkane (dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, or monobromodecane) was poured into the flask as a solvent for dissolution. 1 equivalent of a Lewis acid catalyst (p-toluenesulfonic acid or triflic acid or boron trifluoride diethyl ether or ferric trichloride or aluminum trichloride) was added with stirring. The reaction was monitored by thin-layer chromatography. After 2 h, the reaction was completed. The reaction mixture was quenched with 100 mL of a saturated solution of sodium bicarbonate, washed with 50 mL of a saturated solution of sodium chloride, and dried over anhydrous sodium sulfate. The resulting mixture was subjected to silica gel column chromatography to isolate a macrocyclic arene 14 trimeric ring formation product.

Trimer: ¹H NMR (400 MHz, CDCl₃) δ 7.49 (12H), 7.44 (12H), 7.11 (6H), 6.96 (6H), 6.56 (6H), 3.92 (6H), 3.87 (18H), 3.82 (18H).

(15) Preparation of Macrocyclic Arene 15

To a 500 mL round-bottomed flask were added 1 g of monomer M13 and 0.2 g of paraformaldehyde, and 150 mL of a haloalkane (dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, or monobromodecane) was poured into the flask as a solvent for dissolution. 1.5 equivalents of a Lewis acid catalyst (p-toluenesulfonic acid or triflic acid or boron trifluoride diethyl ether or ferric trichloride or aluminum trichloride) was added with stirring. The reaction was monitored by thin-layer chromatography. After 30 min, the reaction was completed. The reaction mixture was quenched with 100 mL of a saturated solution of sodium bicarbonate, washed with 50 mL of a saturated solution of sodium chloride, and dried over anhydrous sodium sulfate. The resulting mixture was subjected to silica gel column chromatography to isolate a macrocyclic arene 15 trimeric ring formation product.

Trimer: ¹H NMR (600 MHz, DMSO-d₆) δ 6.80 (12H), 3.87 (18H), 3.84 (6H), 3.78 (18H).

(16) Preparation of Macrocyclic Arene 16

To a 500 mL round-bottomed flask were added 0.5 g of monomer M14 and 0.4 g of paraformaldehyde, and 300 mL of a haloalkane (dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, or monobromodecane) was poured into the flask as a solvent for dissolution. 4-fold equivalents of a Lewis acid catalyst (p-toluenesulfonic acid or triflic acid or boron trifluoride diethyl ether or ferric trichloride or aluminum trichloride) was added with stirring. The reaction was monitored by thin-layer chromatography. After 3 h, the reaction was completed. The reaction mixture was quenched with 100 mL of a saturated solution of sodium bicarbonate, washed with 50 mL of a saturated solution of sodium chloride, and dried over anhydrous sodium sulfate. The resulting mixture was subjected to silica gel column chromatography to isolate a macrocyclic arene 16 trimeric ring formation product.

Trimer: ¹H NMR (500 MHz, CDCl₃) δ 8.35 (6H), 8.25 (6H), 7.86 (6H), 7.01 (6H), 6.60 (6H), 3.95 (6H), 3.93 (18H), 3.86 (18H); HRMS (MALDI-TOF): m/z calcd. for C₉₃H₇₂O₁₈Na⁺ [M+Na]⁺, 1500.4644; found: 1500.4692.

(17) Preparation of Macrocyclic Arene 17

To a 500 mL round-bottomed flask were added monomer M15 and 0.2 g of paraformaldehyde, and 200 mL of a haloalkane (dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, or monobromodecane) was poured into the flask as a solvent for dissolution. 3-fold equivalents of a Lewis acid catalyst (p-toluenesulfonic acid or triflic acid or boron trifluoride diethyl ether or ferric trichloride or aluminum trichloride) was added with stirring. The reaction was monitored by thin-layer chromatography. After 40 min, the reaction was completed. The reaction mixture was quenched with 100 mL of a saturated solution of sodium bicarbonate, washed with 50 mL of a saturated solution of sodium chloride, and dried over anhydrous sodium sulfate. The resulting mixture was subjected to silica gel column chromatography to isolate macrocyclic arene 17 trimeric, tetrameric and pentameric ring formation products.

Trimer: ¹H NMR (500 MHz, CDCl₃) δ 7.82 (6H), 7.77 (6H), 7.59 (6H), 7.05 (6H), 6.60 (6H), 3.99 (6H), 3.90 (18H), 3.80 (18H); HRMS (ESI): m/z calcd. for C₈₁H₇₂O₁₂⁺ [M+NH₄]⁺, 1254.5346, found: 1254.5340.

Tetramer: ¹H NMR (500 MHz, CDCl₃) δ 7.79 (8H), 7.74 (8H), 7.56 (8H), 7.03 (8H), 6.58 (8H), 3.96 (8H), 3.90 (24H), 3.77 (24H); HRMS (ESI): m/z calcd. for C₁₀₈H₉₆O₁₆⁺ [M+NH₄]⁺, 1666.7059, found: 1666.7056.

Pentamer: ¹H NMR (500 MHz, CDCl₃) δ 7.86 (10H), 7.79 (10H), 7.62 (10H), 7.19 (10H), 6.57 (10H), 3.98 (10H), 3.90 (30H), 3.77 (30H); HRMS (ESI): m/z calcd. for C₁₃₅H₁₂₀O₂₀⁺ [M+NH₄]⁺, 2078.8706, found: 2079.8706.

(18) Preparation of a Hexamethoxybiphenyl Macrocyclic Arene

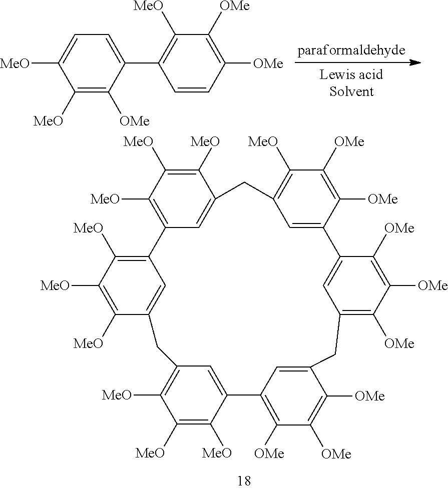

To a 500 mL round-bottomed flask were added 1 g of monomer M27 (CAS: 6322-17-4) and 0.1 g of paraformaldehyde, and 100 mL of a haloalkane (dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, or monobromodecane) was poured into the flask as a solvent for dissolution. 0.5 equivalents of a Lewis acid catalyst (p-toluenesulfonic acid or triflic acid or boron trifluoride diethyl ether or ferric trichloride or aluminum trichloride) was added with stirring. The reaction was monitored by thin-laver chromatography. After 30 min, the reaction was completed. The reaction mixture was quenched with 100 mL of a saturated solution of sodium bicarbonate, washed with 50 mL of a saturated solution of sodium chloride, and dried over anhydrous sodium sulfate. The resulting mixture was subjected to silica gel column chromatography to isolate a macrocyclic arene 18.

¹H NMR (500 MHz, CDCl₃): δ 6.53 (6H), 3.92 (6H), 3.90 (18H), 3.80 (18H), 3.60 (18H); HRMS (ESI): m/z calcd. for C₅₇H₆₇O₁₈⁺ [M+H]⁺, 1039.4327; found: 1039.4326.

[2] Dimeric Supramolecular Macrocycles Prepared with V-Shaped Molecules

The synthesis route is as follows:

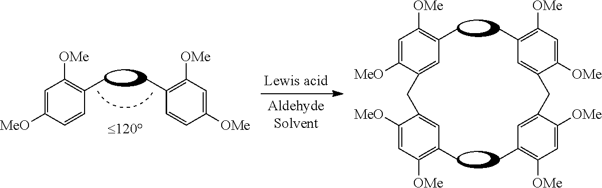 [SMILES: COc1ccc(C)c(OC)c1 ; COc1ccc(C)c(OC)c1 ; COc1cc(OC)c(Cc2cc(C)c(OC)cc2OC)cc1C ; COc1cc(OC)c(Cc2cc(C)c(OC)cc2OC)cc1C]

| 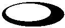 | Reactant | Solvent | Lewis acid | Side chain | No. |
|---|---|---|---|---|---|
| 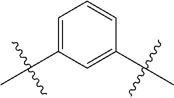 [SMILES: *c1cccc(*)c1] 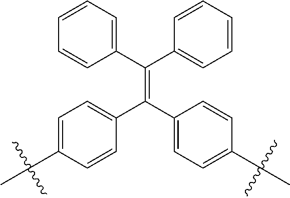 [SMILES: *c1ccc(C(=C(c2ccccc2)c2ccccc2)c2ccc(*)cc2)cc1] 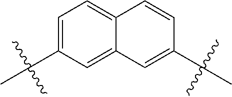 [SMILES: *c1ccc2ccc(*)cc2c1] | Paraformaldehyde Paraformaldehyde Paraformaldehyde | Dichloro/ bromomethane Trichloro/ bromomethane Tetrachloromethane Dichloro/bromoethane Trichloro/bromoethane Tetrachloro/ bromoethane Monochloro/ bromopropane Monochloro/ bromobutane Monochloro/ bromopentane Monochloro/ bromobexane Monochloro/ bromoheptane Monochloro/ bromooctane Monochloro/ bromononane Monochloro/ bromodecane (The solvents above are all reactive) | p-Toluenesulfonic acid Triflic acid Boron trifluoride diethyl ether Ferric chloride Aluminium trichloride (The Lewis acids above can all catalyze a reaction) | 2 2 2 | 19 20 21 |
| 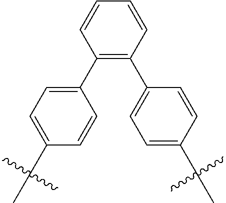 [SMILES: *c1ccc(-c2ccccc2-c2ccc(*)cc2)cc1] | Paraformaldehyde | | | 2 | 22 |
| 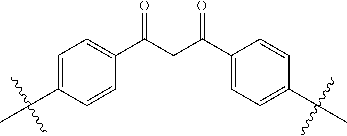 [SMILES: *c1ccc(C(=O)CC(=O)c2ccc(*)cc2)cc1] | Paraformaldehyde | | | 2 | 23 |
| 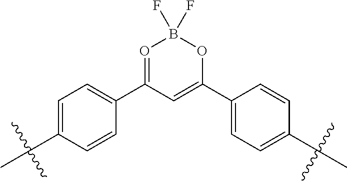 | Paraformaldehyde | | | 2 | 24 |
| 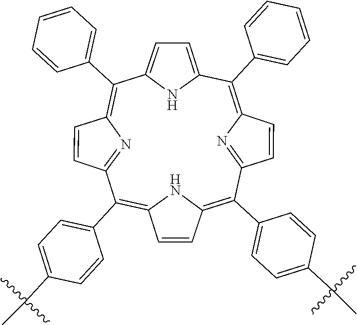 [SMILES: *c1ccc(-c2c3nc(c(-c4ccccc4)c4ccc([nH]4)c(-c4ccccc4)c4nc(c(-c5ccc(*)cc5)c5ccc2[nH]5)C=C4)C=C3)cc1] | Paraformaldehyde | | | 2 | 25 |

(1) Preparation of Macrocyclic Arene 19

To a 500 mL round-bottomed flask were added 0.2 g of monomer M16 and 0.02 g of paraformaldehyde, and 150 ml, of a haloalkane (dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, or monobromodecane) solvent was poured into the flask for dissolution. 1 equivalent of a Lewis acid catalyst (p-toluenesulfonic acid or triflic acid or boron trifluoride diethyl ether or ferric trichloride or aluminum trichloride) was added with stirring. The reaction was monitored by thin-layer chromatography. After 30 min, the reaction was completed. The reaction mixture was quenched with 100 mL of a saturated solution of sodium bicarbonate, washed with 50 mL of a saturated solution of sodium chloride, and dried over anhydrous sodium sulfate. The resulting mixture was subjected to silica gel column chromatography to isolate a macrocyclic arene 19 dimeric ring formation product.

¹H NMR (400 MHz, CDCl₃) δ 7.54 (4H), 7.32 (2H), 7.10 (2H), 6.83 (4H), 6.55 (4H), 3.84 (12H), 3.82 (16H); HRMS (MALDI-TOF): m/z calcd. for C₄₆H₄₄NaO₈⁺ [M+Na]⁺, 747.2928; found: 747.2988.

(2) Preparation of Macrocyclic Arene 20

To a 500 mL round-bottomed flask were added 1 g of monomer M17 and 0.3 g of paraformaldehyde, and 150 mL of a haloalkane (dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, or monobromodecane) solvent was poured into the flask for dissolution. 1.5 equivalents of a Lewis acid catalyst (p-toluenesulfonic acid or triflic acid or boron trifluoride diethyl ether or ferric trichloride or aluminum trichloride) was added with stirring. The reaction was monitored by thin-layer chromatography. After 30 min, the reaction was completed. The reaction mixture was quenched with 100 mL of a saturated solution of sodium bicarbonate, washed with 50 mL of a saturated solution of sodium chloride, and dried over anhydrous sodium sulfate. The resulting mixture was subjected to silica gel column chromatography to isolate a macrocyclic arene 20 dimeric ring formation product.

¹H NMR (500 MHz, CDCl₃) δ 7.17 (8H), 7.09-7.02 (20H), 6.98 (8H), 6.90 (4H), 6.52 (4H), 3.85 (4H), 3.84 (12H), 3.75 (12H); HRMS (ESI): m/z calcd. for C₈₆H₇₂NaO₈⁺ [M+Na]⁺, 1255.5119; found: 1255.5128.

(3) Preparation of Macrocyclic Arene 21

To a 500 mL round-bottomed flask were added monomer M18 and 0.5 g of paraformaldehyde, and 200 mL of a haloalkane (dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, or monobromodecane) solvent was poured into the flask for dissolution. 2 equivalents of a Lewis acid catalyst (p-toluenesulfonic acid or triflic acid or boron trifluoride diethyl ether or ferric trichloride or aluminum trichloride) was added with stirring. The reaction was monitored by thin-layer chromatography. After 15 min, the reaction was completed. The reaction mixture was quenched with 100 mL of a saturated solution of sodium bicarbonate, washed with 50 mL of a saturated solution of sodium chloride, and dried over anhydrous sodium sulfate. The resulting mixture was subjected to silica gel column chromatography to isolate a macrocyclic arene 21 dimeric ring formation product.

¹H NMR (500 MHz, CDCl₃) δ 7.79 (4H), 7.75-7.70 (4H), 7.67 (4H), 7.01 (4H), 6.58 (4H), 3.95 (4H), 3.88 (12H), 3.79 (12H); HRMS (ESI): m/z calcd. for C₅₄H₄₈O₈⁺ [M+H]⁺, 825.3422, found: 825.3425.

(4) Preparation of Macrocyclic Arene 22

To a 500 mL round-bottomed flask were added 0.3 g of monomer M19 and 0.5 g of paraformaldehyde, and 300 mL of a haloalkane (dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, or monobromodecane) solvent was poured into the flask for dissolution. 2 equivalents of a Lewis acid catalyst (p-toluenesulfonic acid or triflic acid or boron trifluoride diethyl ether or ferric trichloride or aluminum trichloride) was added with stirring. The reaction was monitored by thin-layer chromatography. After 20 min, the reaction was completed. The reaction mixture was quenched with 100 mL of a saturated solution of sodium bicarbonate, washed with 50 mL of a saturated solution of sodium chloride, and dried over anhydrous sodium sulfate. The resulting mixture was subjected to silica gel column chromatography to isolate a macrocyclic arene 22 dimeric ring formation product.

¹H NMR (400 MHz, CDCl₃) δ 7.45 (4H), 7.38 (4H), 7.32 (8H), 7.13 (8H), 6.87 (4H), 6.53 (4H), 3.85 (12H), 3.84 (4H), 3.78 (12H); HRMS (ESI): m/z calcd. for C₇₀H₆₀O₈Na⁺ [M+Na]⁺, 1051.4186 found: 1051.4180.

(5) Preparation of Macrocyclic Arene 23

To a 500 mL round-bottomed flask were added 0.2 g of monomer M20 and 70 mg of paraformaldehyde, and 200 mL of a haloalkane (dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, or monobromodecane) solvent was poured into the flask for dissolution. 3 equivalents of a Lewis acid catalyst (p-toluenesulfonic acid or triflic acid or boron trifluoride diethyl ether or ferric trichloride or aluminum trichloride) was added with stirring. The reaction was monitored by thin-layer chromatography. After 40 min, the reaction was completed. The reaction mixture was quenched with 100 mL of a saturated solution of sodium bicarbonate, washed with 50 mL of a saturated solution of sodium chloride, and dried over anhydrous sodium sulfate. The resulting mixture was subjected to silica gel column chromatography to isolate a macrocyclic arene 23 dimeric ring formation product.

¹H NMR (500 MHz, CDCl₃) δ 8.00 (8H), 7.82 (2H), 7.57 (8H), 7.00 (4H), 6.91 (2H), 6.56 (4H), 3.94 (4H), 3.89 (12H), 3.82 (12H); HRMS (ESI): m/z calcd. for C₃₁H₂₈O₆⁺ [M+H]⁺, 497.1959; found: 497.1962.

(6) Preparation of Macrocyclic Arene 24

To a 500 mL round-bottomed flask were added 0.2 g of monomer M21 and 70 mg of paraformaldehyde, and 200 mL of a haloalkane (dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, or monobromodecane) solvent was poured into the flask for dissolution. 3 equivalents of a Lewis acid catalyst (p-toluenesulfonic acid or triflic acid or boron trifluoride diethyl ether or ferric trichloride or aluminum trichloride) was added with stirring. The reaction was monitored by thin-layer chromatography. After 40 min, the reaction was completed. The reaction mixture was quenched with 100 mL of a saturated solution of sodium bicarbonate, washed with 50 mL of a saturated solution of sodium chloride, and dried over anhydrous sodium sulfate. The resulting mixture was subjected to silica gel column chromatography to isolate a macrocyclic arene 24 dimeric ring formation product.

¹H NMR (500 MHz, DMSO-d₆) δ 8.36(8H), 7.71 (8H), 7.14(4H), 6.78 (4H), 3.89 (12H), 3.88 (4H), 3.84 (12H); HRMS (ESI): m/z calcd. for C₃₁H₂₇B₁F₂O₆⁺[M+H]⁺, 545.1947; Found: 545.1945.

(7) Preparation of Macrocyclic Arene 25

To a 500 mL round-bottomed flask were added monomer M22 and 80 mg of paraformaldehyde, and 200 mL of a haloalkane (dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, or monobromodecane) was poured into the flask as a solvent for dissolution. 3 equivalents of a Lewis acid catalyst (p-toluenesulfonic acid or triflic acid or boron trifluoride diethyl ether or ferric trichloride or aluminum trichloride) was added with stirring. The reaction was monitored by thin-layer chromatography. After 15 min, the reaction was completed. The reaction mixture was quenched with 100 mL of a saturated solution of sodium bicarbonate, washed with 50 mL of a saturated solution of sodium chloride, and dried over anhydrous sodium sulfate. The resulting mixture was subjected to silica gel column chromatography to isolate a macrocyclic arene 25 dimeric ring formation product.

¹H NMR (500 MHz, CDCl₃) δ 9.03 (4H), 8.98-8.97 (4H), 8.83 (8H), 8.22-8.20 (16H), 7.93-7.92 (8H), 7.77-7.42 (12H), 7.36 (41H), 6.78 (41H), 4.15 (41H), 4.04 (12H), 4.01 (12H), 2.74 (4H), HRMS (ESI): calcd. for C₁₂₂H₉₂N₈O₈⁺ [M+H]⁺, 1798.7144; found: 1798.7145.

[3] Construction of Supracage-Like Molecule Compounds Using Monomer Molecules Having Three 2,4-dimethoxyphenyl Groups

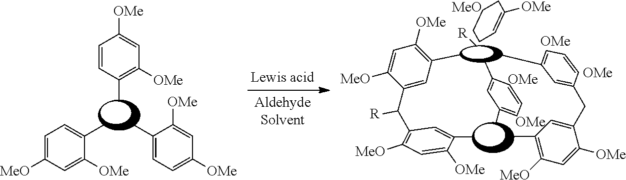

| 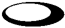 | Reactant | Solvent | Lewis acid | R | No. |
|---|---|---|---|---|---|
| 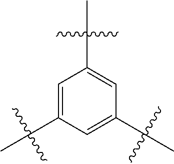 [SMILES: *c1cc(*)cc(*)c1] 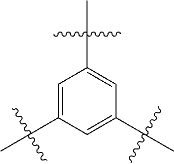 [SMILES: *c1cc(*)cc(*)c1] 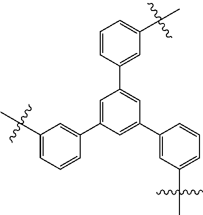 [SMILES: *c1cccc(-c2cc(-c3cccc(*)c3)cc(-c3cccc(*)c3)c2)c1] | Paraformaldehyde 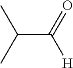 Paraformaldehyde | Dichloro/ bromomethane Trichloro/ bromomethane Tetrachloromethane Diehl oro/bromoethane Trichloro/bromoethane Tetrachloro/ bromoethane Monochloro/ bromopropane Monochloro/ bromobutane Monochloro/ bromopentane Monochloro/ bromohexane Monochloro/ bromoheptane Monochloro/ bromooctane Monochloro/ bromononane Monochloro/ bromodecane (The solvents above are all reactive) | p- Toluenesulfonic acid Triflic acid Boron trifluoride diethyl ether Ferric chloride Aluminium trichloride (The Lewis acids above can all catalyze a reaction) | H 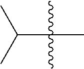 [SMILES: *C(C)C] H | 26 27 28 |

(1) Preparation of Cage-Like Molecule 26

To a 500 mL round-bottomed flask were added 2 g of monomer M23 and 0.8 g of paraformaldehyde, and 150 mL of a haloalkane (dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, or monobromodecane) was poured into the flask for dissolution. 4 equivalents of a Lewis acid catalyst (p-toluenesulfonic acid or triflic acid or boron trifluoride diethyl ether or ferric trichloride or aluminum trichloride) was added with stirring. The reaction was monitored by thin-layer chromatography. After 12 h, the reaction was completed. The reaction mixture was quenched with 200 mL of a saturated solution of sodium bicarbonate, washed with 100 mL of a saturated solution of sodium chloride, and dried over anhydrous sodium sulfate. The resulting mixture was subjected to silica gel column chromatography to isolate a dimeric cage-like molecule product 26.

¹H NMR (500 MHz, CDCl₃) δ 7.38 (2H), 6.95 (4H), 6.62 (12H), 3.92 (20H), 3.76 (18H).

(2) Preparation of Cage-Like Molecule 27

To a 500 mL round-bottomed flask were added 2 g of monomer M23 and 1.1 mL of isobutyraldehyde, and 200 mL of a haloalkane (dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, or monobromodecane) was poured into the flask for dissolution. 4 equivalents of a Lewis acid catalyst (p-toluenesulfonic acid or triflic acid or boron trifluoride diethyl ether or ferric trichloride or aluminum trichloride) was added with stirring. The reaction was monitored by thin-layer chromatography. After 12 h, the reaction was completed. The reaction mixture was quenched with 200 mL of a saturated solution of sodium bicarbonate, washed with 100 mL of a saturated solution of sodium chloride, and dried over anhydrous sodium sulfate. The resulting mixture was subjected to silica gel column chromatography to isolate a dimeric cage-like molecule product 27.

¹H NMR (500 MHz, CDCl₃) δ 7.11 (6H), 7.01 (6H), 6.44 (6H), 4.69 (3H), 3.88 (18H), 3.74 (18H), 2.48-2.29 (3H), 0.86 (18H); HRMS (ESI): m/z calcd. for C₇₂H₇₈O₁₂⁺ [M+H]⁺, 1135.5566; found: 1135.5569.

(3) Preparation of Cage-Like Molecule 28

To a 500 mL round-bottomed flask were added 1.5 g of monomer M24 and 0.2 g of paraformaldehyde, and 100 mL of a haloalkane (dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, or monobromodecane) was poured into the flask as a solvent for dissolution. 4 equivalents of a Lewis acid catalyst (p-toluenesulfonic acid or triflic acid or boron trifluoride diethyl ether or ferric trichloride or aluminum trichloride) was added with stirring. The reaction was monitored by thin-layer chromatography. After 12 h, the reaction was completed. The reaction mixture was quenched with 200 mL of a saturated solution of sodium bicarbonate, washed with 100 mL of a saturated solution of sodium chloride, and dried over anhydrous sodium sulfate. The resulting mixture was subjected to silica gel column chromatography to isolate a dimeric cage-like molecule product 28.

¹H NMR (500 MHz, CDCl₃) δ 7.69 (6H), 7.62 (6H), 7.46 (6H), 7.38 (6H), 7.33 (6H), 6.92 (6H), 6.54 (6H), 3.92 (6H), 3.88 (18H), 3.78 (18H).

[4] Obtaining of a Supramolecular Macrocyclic Compound Having Different Units by Regulating Proportions of Different Monomer Molecules to Achieve Copolymerization of the Different Monomers.

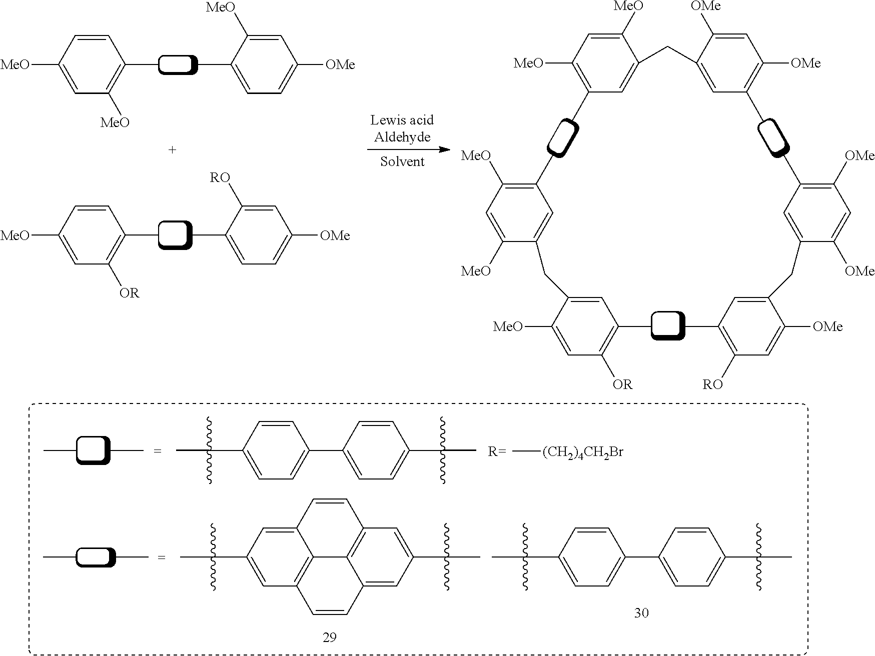

(1) Preparation of Copolymeric Macrocycles Compound 29

To a 500 mL round-bottomed flask were added 0.3 g of monomer M26 and 1 g of monomer M5 in a molar ratio of 1:5, and 200 mL of a haloalkane (dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, or monobromodecane) was added for dissolution. Then 2 equivalents of paraformaldehyde was added. Then 1 equivalent of a Lewis acid catalyst (p-toluenesulfonic acid or triflic acid or boron trifluoride diethyl ether or ferric trichloride or aluminum trichloride) was added with stirring. The reaction was monitored by thin-layer chromatography. After the reaction was completed, the reaction mixture was quenched with a saturated solution of sodium bicarbonate, washed with a saturated solution of sodium chloride, and dried over anhydrous sodium sulfate. The resulting mixture was subjected to silica gel column chromatography to isolate a copolymeric three-membered macrocyclic compound 29.

¹H NMR (400 MHz, CDCl₃) δ 8.25 (4H), 8.23 (4H), 8.01 (8H), 7.61 (8H), 7.52 (4H), 7.27 (2H), 7.23 (2H), 7.02 (2H), 6.66 (4H), 6.56 (2H), 4.08 (2H), 4.00 (4H), 3.95 (4H), 3.95 (6H), 3.91 (6H), 3.89 (6H), 3.86 (6H), 3.84 (6H), 3.33 (4H), 1.82 (4H), 1.72 (4H), 1.52 (4H); HRMS (MALDI-TOF): m/z calcd. for C₁₀₃H₉₂Br₂O₁₂⁺[M]⁺ *, 1680.4935; found: 1680.4959.

(2) Preparation of Copolymeric Macrocyclic Compound 30

To a 500 mL round-bottomed flask were added 0.3 g of monomer M26 and 0.9 g of monomer M2 in a molar ratio of 1:5, and 200 mL of a haloalkane (dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, or monobromodecane) was added for dissolution. Then 2 equivalents of paraformaldehyde was added. Then 1 equivalent of a Lewis acid catalyst (p-toluenesulfonic acid or triflic acid or boron trifluoride diethyl ether or ferric trichloride or aluminum trichloride) was added with stirring. The reaction was monitored by thin-layer chromatography. After the reaction was completed, the reaction mixture was quenched with a saturated solution of sodium bicarbonate, washed with a saturated solution of sodium chloride, and dried over anhydrous sodium sulfate. The resulting mixture was subjected to silica gel column chromatography to isolate a copolymeric three-membered macrocyclic compound 30.

¹H NMR (400 MHz, CDCl₃) δ 7.73-7.57 (12H), 7.55-7.48 (12H), 7.02 (6H), 6.59 (4H), 6.57 (2H), 3.98 (4H), 3.94 (6H), 3.89 (12H), 3.87 (6H), 3.85 (12H), 3.36 (4H), 1.84 (4H), 1.75 (4H), 1.59 (4H); HRMS (MALDI-TOF): m/z calcd. for C₉₅H₉₂Br₂O₁₂⁺ [M]⁺, 1584.4935; found: 1584.4955.

III A derivatization method for the supramolecular macrocyclic and cage-like molecule compound based on biphen[n]arene of step 3 is as follows:

[1] Synthesis of Hydroxyl Compounds

(1) Synthesis of a Trimeric Macrocyclic Hydroxyl Derivative Compound Formed from Linear Monomers

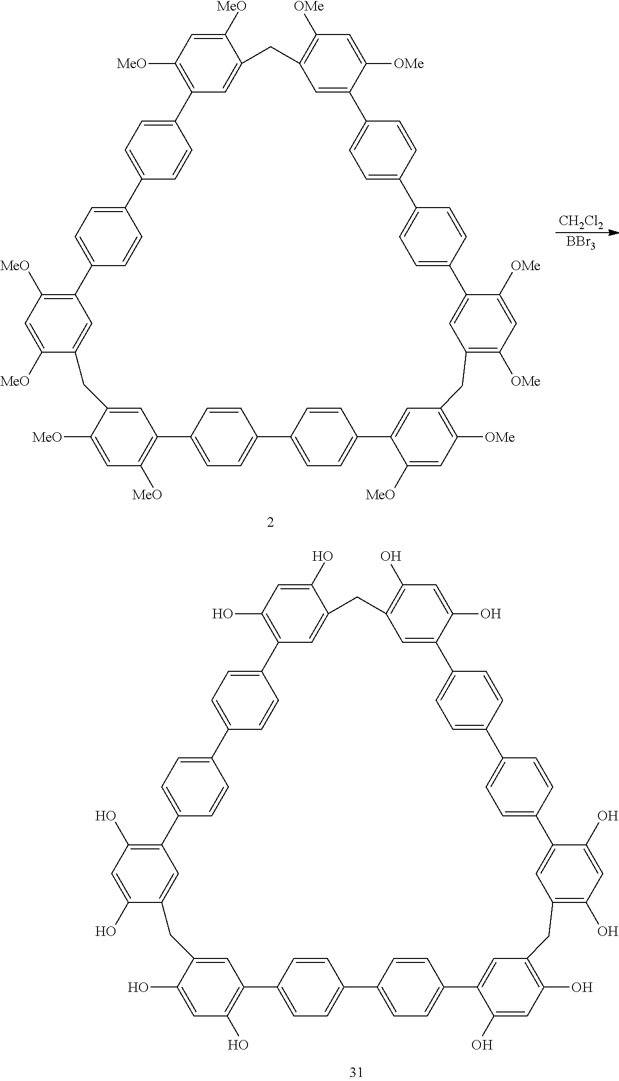 [SMILES: COc1cc(OC)c2cc1Cc1cc(c(OC)cc1OC)-c1ccc(cc1)-c1ccc(cc1)-c1cc(c(OC)cc1OC)Cc1cc(c(OC)cc1OC)-c1ccc(cc1)-c1ccc(cc1)-c1cc(c(OC)cc1OC)Cc1cc(c(OC)cc1OC)-c1ccc(cc1)-c1ccc-2cc1 ; "CH2Cl2" ; "BBr3" ; Oc1cc(O)c2cc1Cc1cc(c(O)cc1O)-c1ccc(cc1)-c1ccc(cc1)-c1cc(c(O)cc1O)Cc1cc(c(O)cc1O)-c1ccc(cc1)-c1ccc(cc1)-c1cc(c(O)cc1O)Cc1cc(c(O)cc1O)-c1ccc(cc1)-c1ccc-2cc1]

Macrocyclic arene 2 was added to a 100 mL round-bottomed flask and dissolved in dichloromethane. 20 equivalents of boron tribromide compound was added to the reaction system. After 1 day of reaction, the reaction mixture was added dropwise to a mixture of ice and water to precipitate a light purple powder, and subjected to suction filtration to obtain a brown macrocyclic product 31.

¹H NMR (400 MHz, acetone-d₆) δ 7.76-7.52 (24H), 7.24 (6H), 6.59 (6H), 3.93 (6H); HRMS (MALDI-TOF): calcd. for C₉₅H₉₂Br₂O₁₂⁺ [M+Na]⁺, 1169.3507, found: 1169.3509.

(2) Synthesis of Dimeric Macrocyclic Hydroxyl Compounds Formed from V-Shaped Compounds

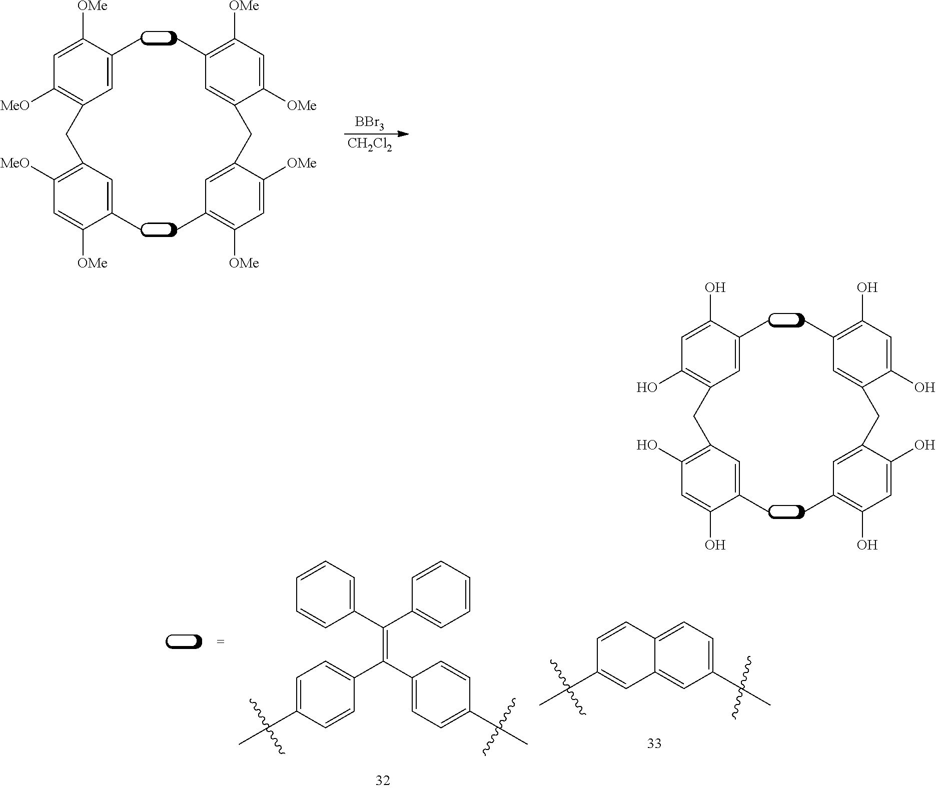

[1] Synthesis of a Hydroxyl Derivative Macrocycle 32

Macrocyclic arene 20 was added to a 100 mL round-bottomed flask and dissolved in dichloromethane. 20 equivalents of boron tribromide compound was added to the reaction system. After 1 day of reaction, the reaction mixture was added dropwise to a mixture of ice and water to precipitate a white solid. Suction filtration was performed to obtain a macrocyclic product 32.

¹H NMR (400 MHz, acetone-d₆) δ 7.36 (8H), 7.27 (4H), 7.15-7.02 (20H), 6.98 (8H), 6.52 (4H), 3.85 (4H); HRMS (ESI): m/z calcd. for C₇₈H₅₆O₈⁺ [M+Na]⁺, 1143.3867; found: 1143.3860.

[2] Synthesis of a Hydroxyl Derivative Macrocycle 33

0.5 g of macrocyclic arene 21 was added to a 100 mL round-bottomed flask and dissolved in dichloromethane. 20 equivalents of boron tribromide compound was added to the reaction system. After 1 day of reaction, the reaction mixture was added dropwise to a mixture of ice and water to precipitate a white solid. Suction filtration was performed to obtain a macrocyclic product 33.

¹H NMR (500 MHz, DMSO-d₆) δ 9.34 (4H), 9.23 (4H), 7.85 (4H), 7.80 (4H), 7.73 (4H), 6.95 (4H), 6.52 (4H), 3.74 (4H); HRMS (ESI): m/z calcd. for C₄₆H₃₂O₈⁺ [M+H]⁺, 713.2164; found: 713.2156.

(3) Synthesis of a Cage-Like Molecule Hydroxyl Derivative Compound

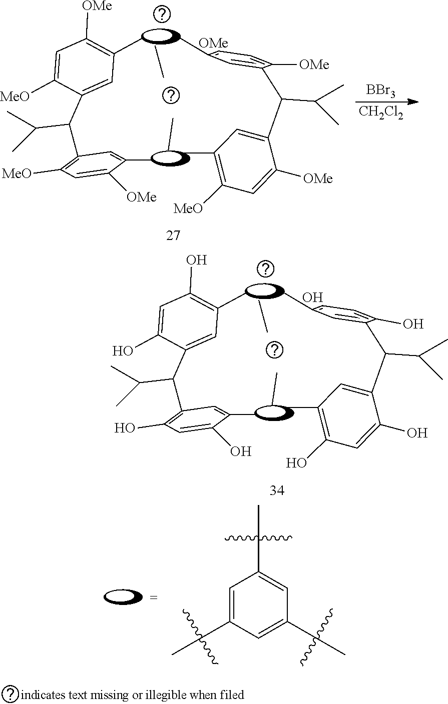

Cage-like molecule compound 27 was added to a 100 mL round-bottomed flask and dissolved in dichloromethane. 40 equivalents of boron tribromide compound was added to the reaction system. After 2 day of reaction, the reaction mixture was added dropwise to a mixture of ice and water to precipitate a white solid. Suction filtration was performed to obtain an isopropyl-cage-like molecule hydroxyl derivative macrocyclic product 34.

¹H NMR (500 MHz, DMSO-d₆) δ 7.96 (12H), 7.23 (12H), 6.88 (6H), 6.45 (6H), 4.33 (3H), 2.29 (3H), 0.89 (18H); HRMS (ESI): m/z calcd. for C₆₀H₅₄O₁₂⁺ [M+H]⁺, 967.3688; found: 967.3688.

[2] Synthesis of a Carboxylic Acid Water-Soluble Macrocyclic and Cage-Like Molecule

(1) Synthesis of a Macrocyclic Ammonium Carboxylate Salt Water-Soluble Macrocycle 35

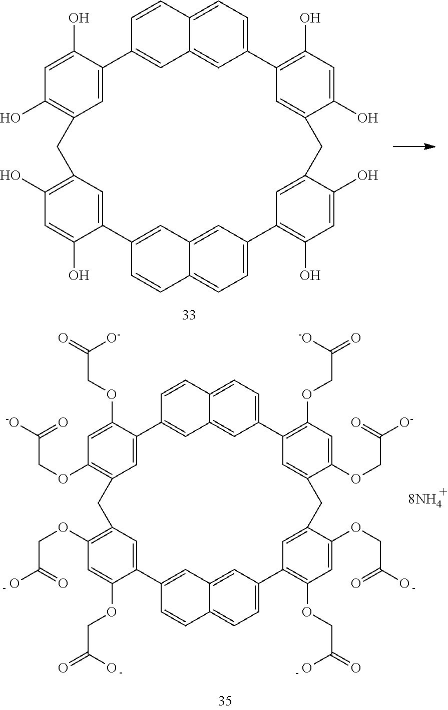

In a 100 mL round-bottomed flask, 0.9 g of hydroxyl derivative macrocycle product 33 was dissolved in 50 mL of acetonitrile, and then 3 g of K₂CO was added. The mixed system was refluxed for 8 h. 13 mL of ethyl bromoacetate was then added. The mixed system was refluxed for another 96 h. After the reaction was completed, the reaction mixture was cooled to room temperature, filtered, and washed with dichloromethane multiple times. The solvent was removed by vacuum rotary evaporation. A small amount of dichloromethane was added so that the solid was just dissolved. Then a large amount of petroleum ether was added, with a large amount of solid subsequently precipitated. Suction filtration was performed under reduced pressure to obtain the target product. A carbethoxy-substituted macrocycle was dissolved in a mixed solution of 50 mL of THF and 20 mL of an aqueous solution of sodium hydroxide (a mass concentration of 20%), and the mixture was stirred at reflux for 10 h. THF was removed by rotary evaporation. Then 20 mL of water was added, followed by hydrochloric acid for acidification until a pH test paper showed weak acidity. In the process, a large amount of rufous solid precipitated. The rufous solid was collected by suction filtration and added to 20 mL of aqueous ammonia (mass fraction of 25%-28%) solution, and the mixture was stirred at room temperature for 5 h until the rufous solid was dissolved. After the reaction was completed, the solvent was removed by rotary evaporation to obtain a rufous solid, namely the ammonium carboxylate water-soluble macrocycle 35.

¹H NMR (500 MHz, D₂O) δ 7.84 (4H), 7.77 (12H), 7.62 (4H), 7.36 (4H), 6.82 (4H), 6.17 (8H), 5.64 (4H), 4.44 (4H), 4.29 (4H), 3.68 (4H), 3.64 (4H); HRMS (ESI): m/z calcd. for C₆₂H₄₈O₂₄⁺ [M+NH₄]⁺, 1194.2887; found: 1194.2881.

(2) Synthesis of a Macrocyclic Sodium Carboxylate Salt Water-Soluble Macrocycle 36

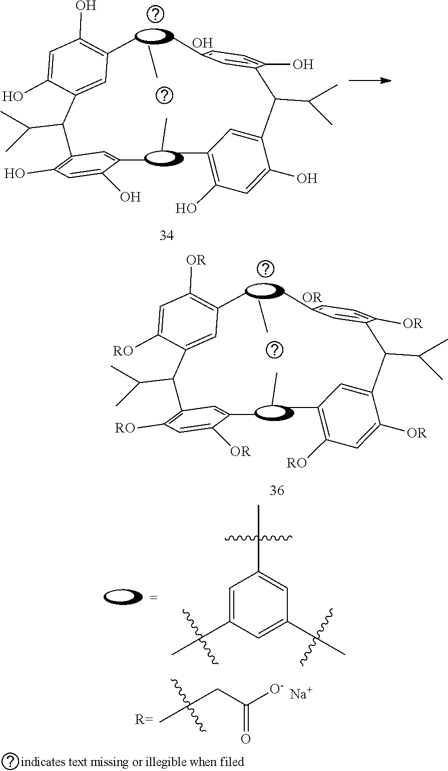

In a 250 mL round-bottomed flask, 0.9 g of isopropyl-cage-like molecule hydroxyl derivative macrocycle product 34 was dissolved in 50 mL of acetonitrile, and then 6 g of K₂CO₃ was added. The mixed system was refluxed for 8 h. 5 mL of ethyl bromoacetate was then added. The mixed system was refluxed for another 96 h. After the reaction was completed, the reaction mixture was cooled to room temperature, filtered, and washed with dichloromethane multiple times. The solvent was removed by vacuum rotary evaporation. A small amount of dichloromethane was added so that the solid was just dissolved. Then a large amount of petroleum ether was added, with a large amount of solid subsequently precipitated. Suction filtration was performed under reduced pressure to obtain the desired product. A carbethoxy-substituted macrocycle was dissolved in a mixed solution of 50 mL of THF and 20 mL of an aqueous solution of sodium hydroxide (a mass concentration of 20%), and the mixture was stirred at reflux for 10 h. THF was removed by rotary evaporation. Then 20 mL of water was added, followed by hydrochloric acid for acidification until a pH test paper showed weak acidity. In the process, a large amount of rufous solid precipitated. The rufous solid was collected by suction filtration and added to 20 mL of sodium hydroxide (a mass concentration of 20%) solution, and the mixture was stirred at room temperature for 8 h until the rufous solid was dissolved. After the reaction was completed, the solvent was removed by rotary evaporation to obtain a white solid, namely the water-soluble sodium carboxylate salt cage-like molecule 36.

¹H NMR (500 MHz, D₂O) δ 7.37 (6H), 7.19 (6H), 6.44 (6H), 4.70 (3H), 4.50 (12H), 4.42 (12H), 2.30 (3H), 0.86 (18H).

[3] Synthesis of Sulfonated Water-Soluble Macrocycles

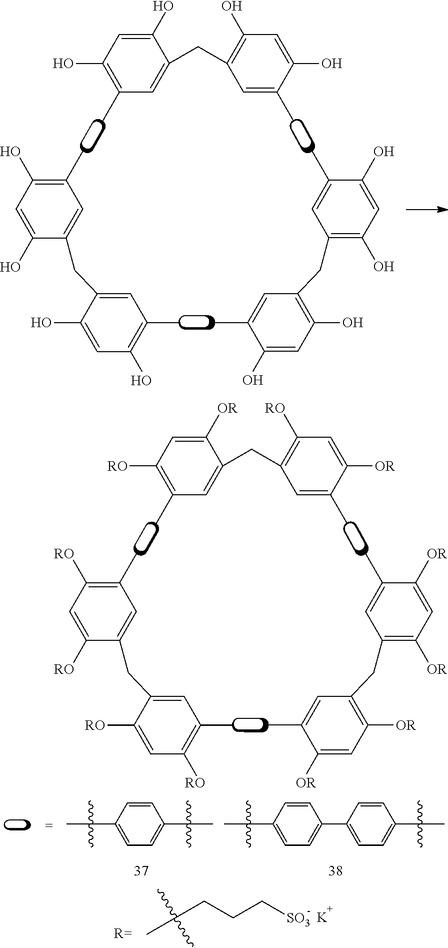

(1) Preparation of a Sulfonic Acid Derivative Macrocycle 37

In a 100 mL round-bottomed flask, the macrocyclic arene compound 1 was first subjected to full-hydroxy derivatization by referring to the method above, and then dissolved in acetone. 7 g of K₂CO₃ was added, and the mixture was stirred at reflux for 2 h. Then 6.5 g of propane sultone (60 equivalents) was added, and the mixture was stirred at reflux for another 3 days. After the reaction was terminated, the reaction mixture was cooled to room temperature, subjected to suction filtration to obtain a filter cake, which was then washed with acetone twice. The obtained filter cake was dissolved in water and purified to remove potassium carbonate by about one week of dialysis with a dialysis bag; 800 mL of double distilled water was added to a 1 L large beaker, and then the dialysis bag was put into the water and slightly fixed with a rubber band; a stirrer was added, and the water was continuously stirred; the water in the beaker was replaced once every 2 hours; the water replacing frequency was reduced after one day to once every half a day; and the water was replaced once on the third day. Finally, the aqueous solution in the dialysis bag was concentrated by rotary evaporation to obtain a sulfonated water-soluble macrocyclic product 37.

¹H NMR (400 MHz, D₂O) δ 7.35 (12H), 6.96 (6H), 6.68 (12H), 4.25-3.90 (22H), 3.82 (6H), 2.80 (24H), 1.99 (24H).

(2) Preparation of a Sulfonic Acid Derivative Macrocycle 38

In a 100 mL round-bottomed flask, 0.9 g of macrocycle 31 was dissolved in acetone. 7 g of K₂CO₃ was added, and the mixture was stirred at reflux for 2 h. Then 6.5 g of propane sultone (60 equivalents) was added, and the mixture was stirred at reflux for another 3 days. After the reaction was terminated, the reaction mixture was cooled to room temperature, subjected to suction filtration to obtain a filter cake, which was then washed with acetone twice. The obtained filter cake was dissolved in water and purified to remove potassium carbonate by about one week of dialysis with a dialysis bag; 800 mL of double distilled water was added to a 1 L large beaker, and then the dialysis bag was put into the water and slightly fixed with a rubber band; a stirrer was added, and the water was continuously stirred; the water in the beaker was replaced once every 2 hours; the water replacing frequency was reduced after one day to once every half a day; and the water was replaced once on the third day. Finally, the aqueous solution in the dialysis bag was concentrated by rotary evaporation to obtain a sulfonated water-soluble macrocyclic product 38.

¹H NMR (400 MHz, DMSO-d₆) δ 7.53 (24H), 6.89 (6H), 6.71 (6H), 4.07 (24H), 3.80 (6H), 2.55 (24H), 1.% (24H).

[4] Special Derivatization of Certain Macrocyclic Compounds

(1) Synthesis of a Carbazole Derivative Macrocycle

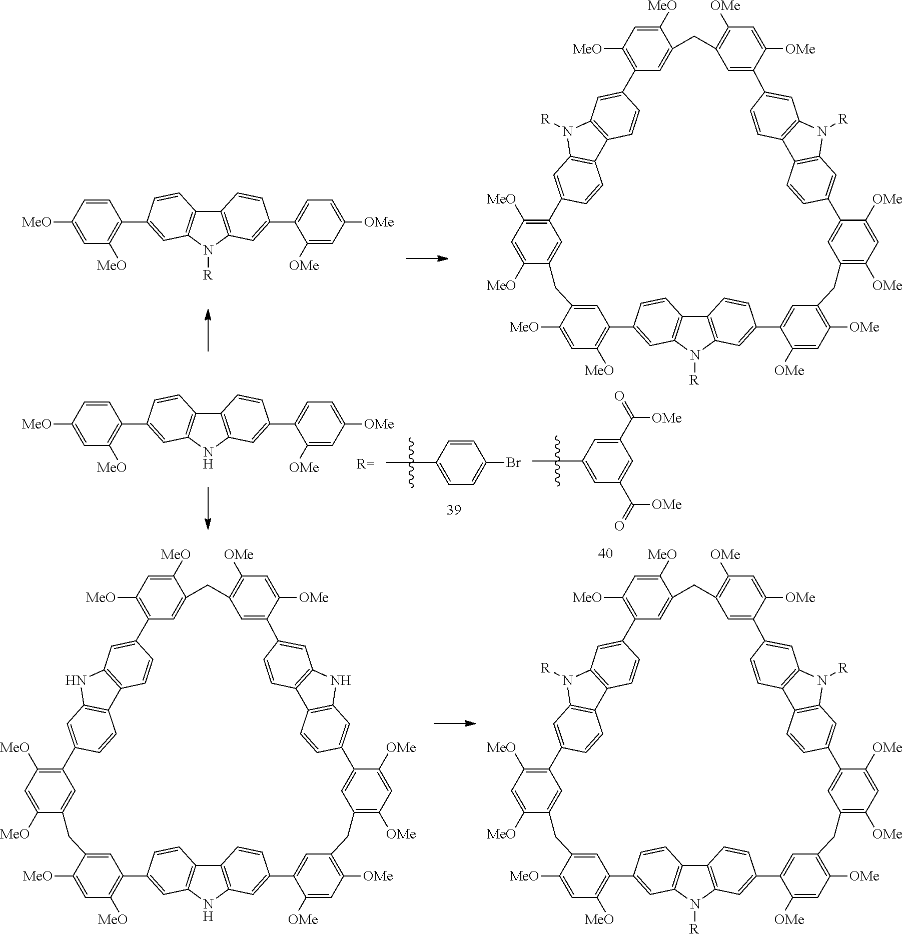

\textcircled{1} Monomer Modification and then Ring Closing

In a 100 mL round-bottomed flask, monomer M9 was mixed with 2 equivalents of p-dibromobenzene (or methyl 5-bromoisophthalate), 1 equivalent of copper(I) iodide and 6 equivalents of potassium carbonate, and the mixture was dissolved in N,N-dimethylacetamide. The resulting solution was heated to 180° C. under nitrogen (argon) atmosphere and allowed to react for 24 h. Then the product was cooled to room temperature, poured into a saturated aqueous solution of NaCl, extracted three times with dichloromethane, dried over anhydrous sodium sulfate, and purified by column chromatography to obtain a white solid product. Subsequently, 3 equivalents of paraformaldehyde was added to the white solid product and a haloalkane (dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, or monobromodecane) was added for dissolution. Then a Lewis acid catalyst (p-toluenesulfonic acid or triflic acid or boron trifluoride diethyl ether or ferric trichloride or aluminum trichloride) was added. The reaction was monitored by thin-layer chromatography. After the reaction was completed, the reaction mixture was quenched with a saturated solution of sodium bicarbonate, washed with a saturated solution of sodium chloride, and dried over anhydrous sodium sulfate. The resulting mixture was subjected to silica gel column chromatography to isolate a three-membered ring macrocyclic product 39 (or 40).

\textcircled{2} Ring Closing, and then Modification

In a 100 mL eggplant-shaped flask, three-membered macrocycle 11 was mixed with 2 equivalents of p-dibromobenzene (or methyl 5-bromoisophthalate), 1 equivalent of copper(I) iodide and 6 equivalents of potassium carbonate, and the mixture was dissolved in N,N-dimethylacetamide. The resulting solution was heated to 180° C. under nitrogen (argon) atmosphere and allowed to react for 24 h. Then the product was cooled to room temperature, poured into a saturated aqueous solution of NaCl, extracted three times with dichloromethane, dried over anhydrous sodium sulfate, and purified by column chromatography to obtain a three-membered ring macrocyclic product 39 (or 40), p-Dibromobenzene modification (three-membered ring macrocyclic product 39): ¹H NMR (400 MHz, CDCl₃) δ 8.01 (6H), 7.39 (12H), 7.38 (12H), 6.99 (6H), 6.55 (6H), 3.92 (6H), 3.87 (18H), 3.78 (18H).

Methyl 5-bromoisophthalate modification (three-membered ring macrocyclic product 40): ¹H NMR (400 MHz, CDCl₃) δ 8.66 (3H), 8.45 (6H), 8.01 (6H), 7.53 (6H), 7.24 (6H), 7.07 (6H), 6.53 (6H), 3.92 (6H), 3.86 (18H), 3.79 (18H), 3.78 (18H).

(2) Synthesis of a Pyridine Derivative Macrocycle

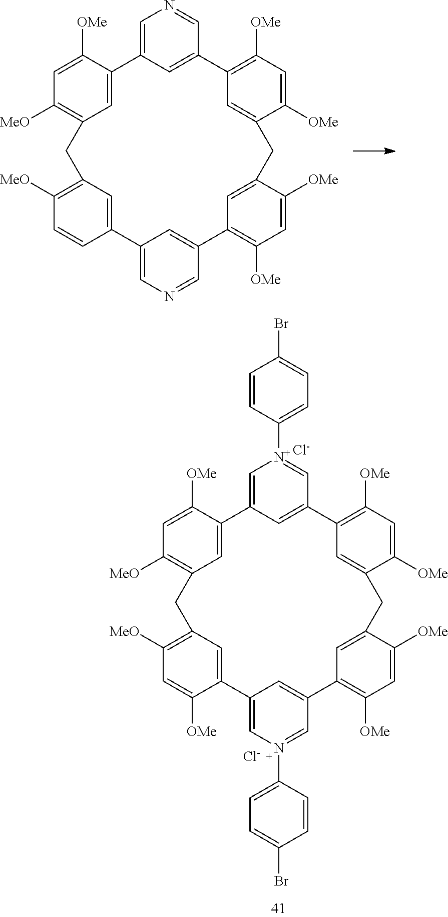

\textcircled{1} Monomer Modification and then Ring Closing

In a 100 mL three-necked flask, 2.4 g of 3,5-dibromopyridine (CAS: 625-92-3) and 3.6 g of 2,4-dimethoxyphenylboronic acid were added to a beaker in a molar ratio of 1:2, and then 2.1 g of anhydrous sodium carbonate and 0.4 g of tetrakis(triphenylphosphine)palladium were added. 80 mL of 1,4-dioxane and 20 mL of water were used as solvents. The resulting mixture was heated at reflux in a 110° C. oil bath overnight. After the reaction was completed, the solvents were sequentially evaporated, extraction was performed with water and dichloromethane, and drying was performed over anhydrous sodium sulfate. The organic phase was separated out. The obtained organic phase was concentrated and purified by column chromatography to isolate a white solid 3,5-bis(2,4-dimethoxyphenyl)pyridine.

¹H NMR (500 MHz, CDCl₃) δ (ppm): 8.78 (4H), 8.35 (2H), 7.07 (4H), 6.72 (4H), 4.31 (6H), 3.90 (12H), 3.87 (12H), 3.84 (4H); HRMS: m/z calcd for C₂₁H₂₂N₁O₄⁺ [M+H]⁺, 353.1543. found: 353.1547.

1.7 g of 3,5-bis-(2,4-dimethoxyphenyl)pyridine monomer and 1.5 g of 2,4-dinitrochlorobenzene were sequentially added to a 50 mL round-bottomed flask, followed by 5 mL of acetone. The mixture was well mixed by ultrasonication, and the reactants were heated at reflux overnight. After the reaction was completed, the solvent was removed by rotary evaporation. A large amount of ethyl acetate was added, and suction filtration was performed. The filter cake was added to acetonitrile solution. Suction filtration was performed, and the filtrate was collected. The filtrate was concentrated by rotary evaporation. A small amount of methanol was added to completely dissolve the residue. Ethyl acetate was added, and the mixture was stirred for 1-4 h. Suction filtration was performed to obtain an intermediate product. 0.5 g of the intermediate product was added to a 50 mL round-bottomed flask, followed by 1 mL of ethanol and 3 mL of water. The mixture was well mixed. Then 3.5 g of p-bromophenylamine was added, and the mixture was heated and refluxed under nitrogen atmosphere for 1-2 days. The mixture was cooled to room temperature. Ethyl acetate was then added and suction filtration was performed. Ethanol was added to the filtrate, and the solvent was removed by rotary evaporation. Then a small amount of acetone was added to dissolve the solid, and a large amount of ethyl acetate was added, with a large amount of solid precipitated. Suction filtration was performed to obtain a modified pyridine arene derivative monomer. Subsequently, 1 g of the derivative arene monomer and 0.3 g of paraformaldehyde were weighed, and 150 mL of a haloalkane (dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, or monobromodecane) was poured as a solvent for dissolution. 1.5 equivalents of a Lewis acid catalyst (p-toluenesulfonic acid or triflic acid or boron trifluoride diethyl ether or ferric trichloride or aluminum trichloride) was added with stirring. The reaction was monitored by thin-layer chromatography. After 30 min, the reaction was completed. The reaction mixture was quenched with 10 mL of a saturated solution of sodium bicarbonate, washed with 50 mL of a saturated solution of sodium chloride, and dried over anhydrous sodium sulfate. The resulting mixture was subjected to silica gel column chromatography to isolate a target product derivative macrocycle 41.

\textcircled{2} Ring Closing, and then Modification.

3.5 g of 3,5-bis(2,4-dimethoxyphenyl)pyridine compound was dissolved in 100 mL of a haloalkane (dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, or monobromodecane), and 0.5 g of paraformaldehyde was added. The mixture was stirred for several minutes until complete dissolution was achieved. Then a Lewis acid catalyst (p-toluenesulfonic acid or triflic acid or boron trifluoride diethyl ether or ferric trichloride or aluminum trichloride) was added, and the reaction mixture gradually turned from colorless to light purple. The mixture was stirred at room temperature for 25 min, and the reaction was monitored by TLC. When the starting materials were substantially consumed, the reaction was quenched with a saturated solution of sodium bicarbonate. The organic phase was separated out, washed once with water, and finally extracted with a saturated solution of NaCl. The organic phase was separated out. The obtained organic phase was concentrated, and the resulting solid was purified by column chromatography (dichloromethane/ethyl acetate, 3:1 v/v) to isolate a compound pyridine derivative macrocycle.

¹H NMR (500 MHz, CDCl₃): δ (ppm): 8.78 (4H), 7.31 (2H), 6.79 (4H), 6.57 (4H), 3.86 (24H), 3.84 (4H); HRMS (ESI): m/z calcd. for C44H43N2O8⁺ [M+H], 727.3017; found: 727.3019.

1.8 g of the pyridine derivative macrocycle and 1.5 g of 2,4-dinitrochlorobenzene were sequentially added to a 50 mL round-bottomed flask, followed by 5 mL of acetone. The mixture was mixed by ultrasonication, and then heated at reflux overnight. And after the reaction was completed, the solvent was removed by rotary evaporation. A large amount of ethyl acetate was added, and suction filtration was performed. The filter cake was dissolved in acetonitrile. Suction filtration was performed, and the filtrate was concentrated by rotary evaporation. The residue was dissolved in a small amount of methanol, and ethyl acetate was added. The mixture was stirred for 1-4 h, and subjected to suction filtration to obtain an intermediate product. 0.5 g of the intermediate product was added to 1 mL of ethanol, followed by 3 mL of water. The mixture was well mixed, and then 3.5 g of p-bromophenylamine was added. The mixture was refluxed under nitrogen atmosphere for 1-2 days. After the reaction was completed, the reaction mixture was cooled to mom temperature. Ethyl acetate was added, and suction filtration was performed. The solvent was removed by rotary evaporation. A small amount of acetone was added to dissolve the solid, and a large amount of ethyl acetate was added to precipitate a solid. Suction filtration was performed to obtain the target product derivative macrocycle 41.

Derivative macrocycle 41: ¹H NMR (600 MHz, CDCl₃) δ 8.98 (4H), 8.52 (2H), 7.90 (8H), 7.33 (4H), 6.57 (4H), 3.93 (16H), 3.89 (12H).

### Example 2

Use of Supramolecular Macrocyclic and Cage-like molecule Compound Based on Biphen[n]arene and Derivative Compounds in Materials, Environment and Biology.

(1) Use of Macrocyclic and Cage-Like Molecule Based on Biphen[n]Arene

[1] Use of Macrocyclic Arene as Adsorptive Separation Material

Trimethylbenzene isomers have very wide application. However, as the boiling points of different trimethylbenzene isomers are very close to each other, trimethylbenzene isomers are very difficult to separate industrially. As the supramolecular macrocycle synthesized by us has a specific cavity structure, trimethylbenzene isomers can be selectively adsorbed and thus separated.

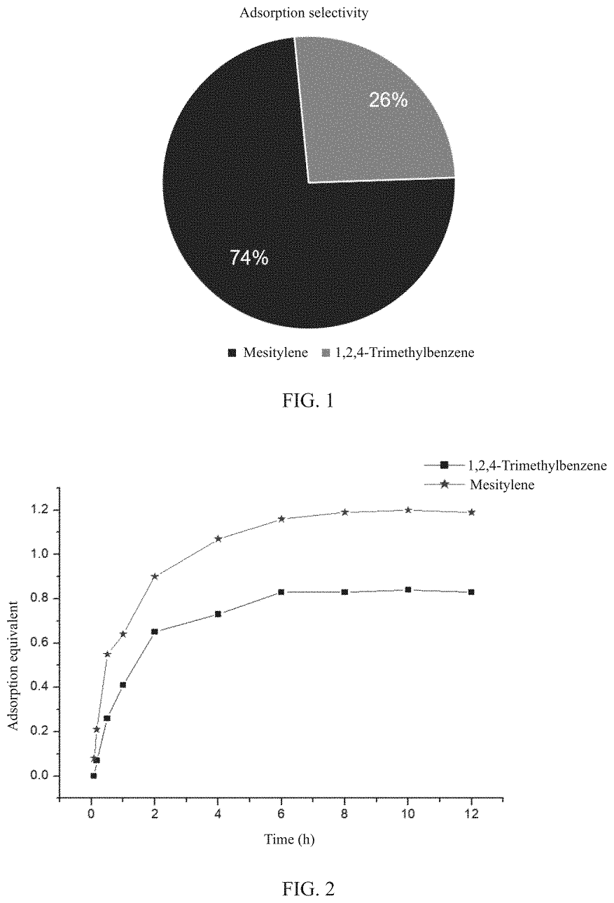

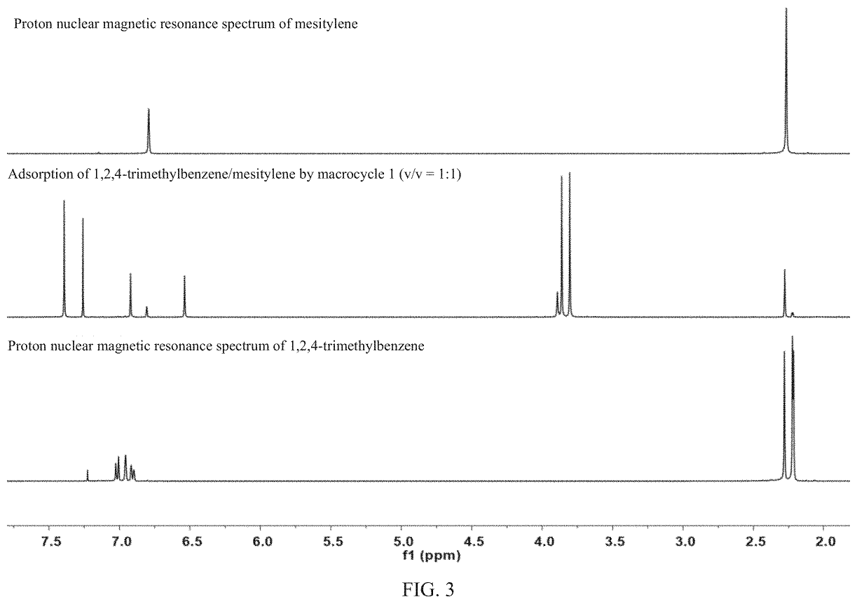

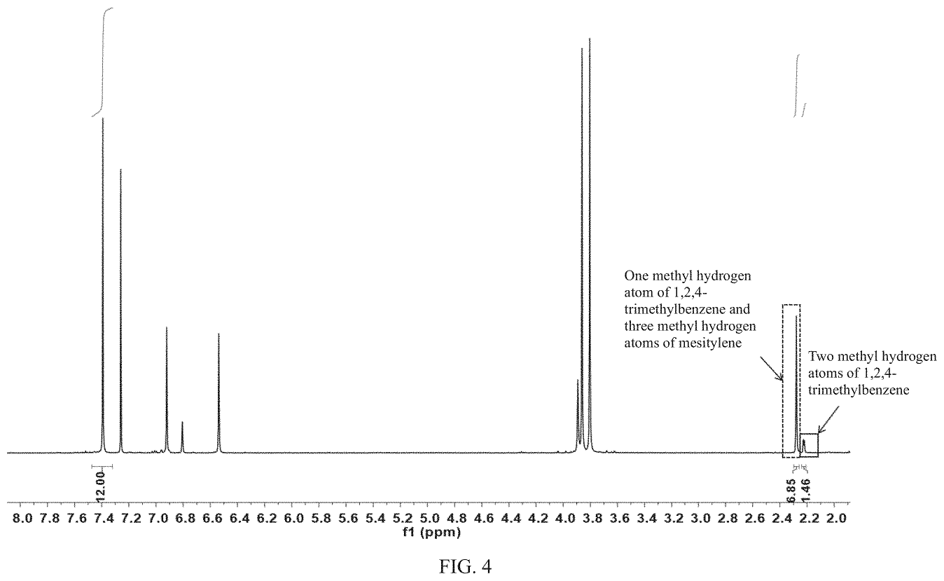

A monocrystal of macrocyclic arene 1 was activated in a vacuum oven (45° C., 2 mmHg pressure) for 8 h, and the material was placed in a saturated mixed vapor of mesitylene and 1,2,4-trimethylbenzene. After 12 h of adsorption, the material was dissolved in deuterated chloroform, and the solution was then characterized by proton nuclear magnetic resonance spectroscopy. The proton nuclear magnetic resonance spectrum shows that the separation ratio (i.e., the adsorption molar ratio of two target adsorption guests converted by integration in the nuclear magnetic resonance spectrum) reaches 74:26, and the separation equivalent of the host (macrocyclic arene 1)/mesitylene/1,2,4-trimethylbenzene=1:0.68:0.24 (amount of substance), demonstrating that macrocyclic arene 1 has the potential to be used as an adsorptive separation material (FIGS. 1-4).

[2] Use of Macrocyclic Arene as Host in Recognition of Ammonium Cationic Compound

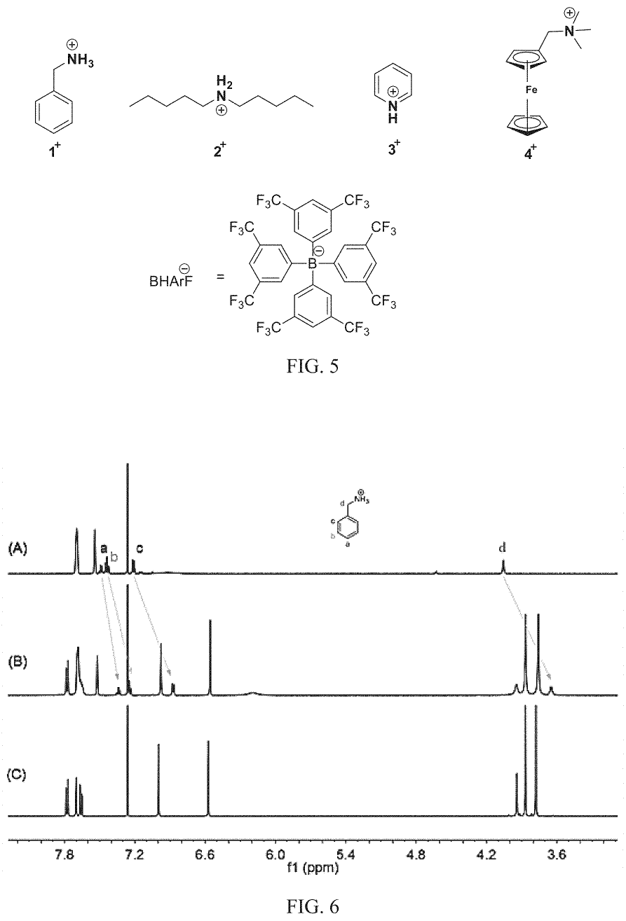

Many amino molecules are important components of organisms, and therefore, the research on the amine cationic compounds is very necessary. According to the studies on the host-guest complexation behavior of various macrocyclic host molecules such as pillararenes and biphenyl arenes, many cations such as quaternary ammonium salt cation guests and secondary ammonium salts are ideal guest molecules. In view of the structural characteristics of our biphenyl arene (host), we selected the following guest molecules to study the host-guest binding properties (FIG. 5).

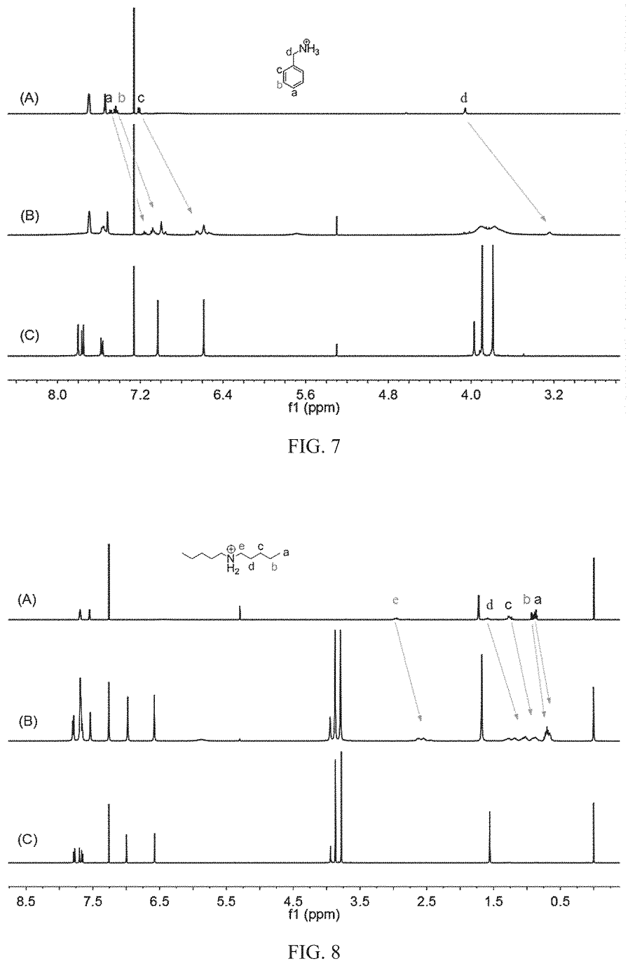

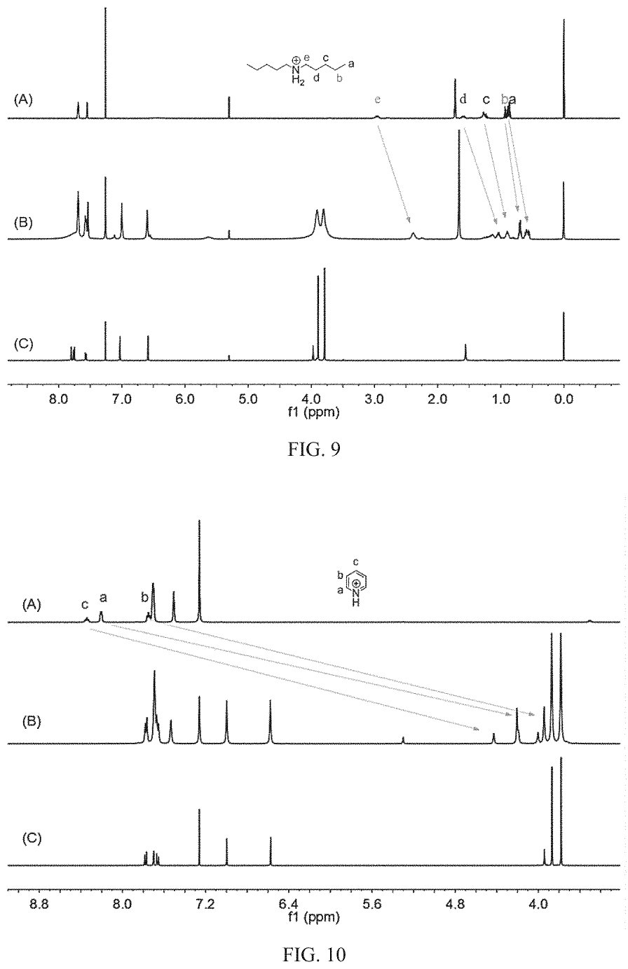

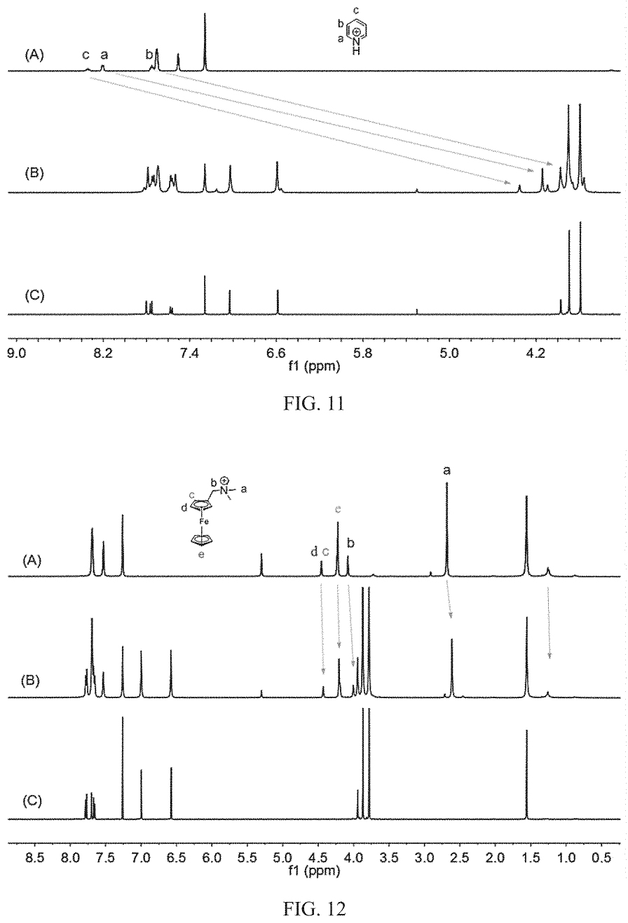

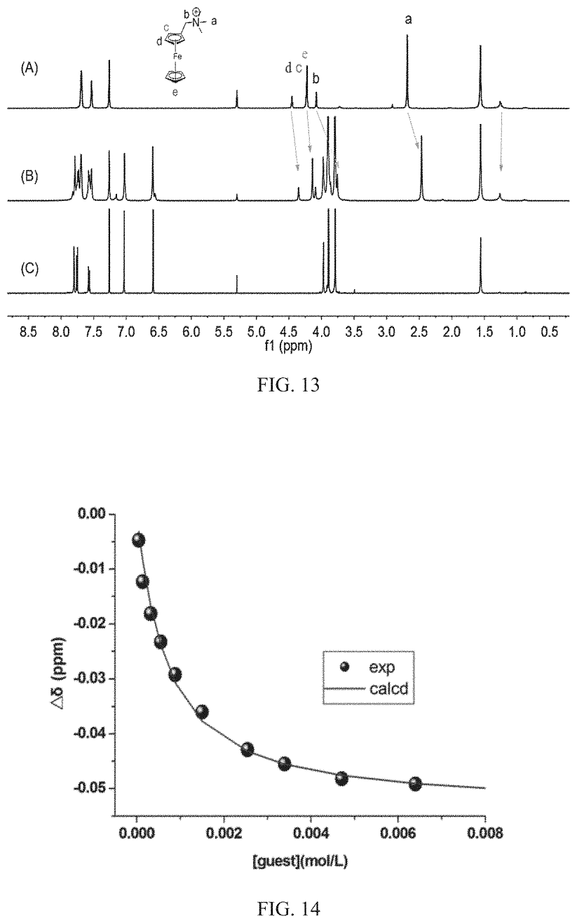

To quantitatively evaluate the binding behavior of a host and a guest, the nuclear magnetic resonance titration method was adopted, i.e., the host concentration was fixed, the guest concentration was changed from low to high levels, and the binding constant was calculated using the non-linear fitting method. The binding constant of macrocyclic arene 17 to guest 1 was (1.74±0.18)×10³ M⁻¹, and the binding constant to guest 2 was (7.63±0.56)×10² M⁻¹; the binding constant of macrocycle 21 to guest 1 was (4.7±0.2)×10² M⁻¹, and the binding constant to guest 2 was (3.64±0.32)×10² M⁻¹ (FIGS. 6-14).

The binding constants were calculated using the methods described in references K. A. Connors. Binding Constants, Wiley: New York, 1987 and X. Shu, S. Chen, J. Li, Z. Chen. L. Weng, X. Jia and C. Li. Chem Commun, 2012, 48, 2967-2969.

[3] Use of Cage-Like Molecule as Adsorptive Separation Material

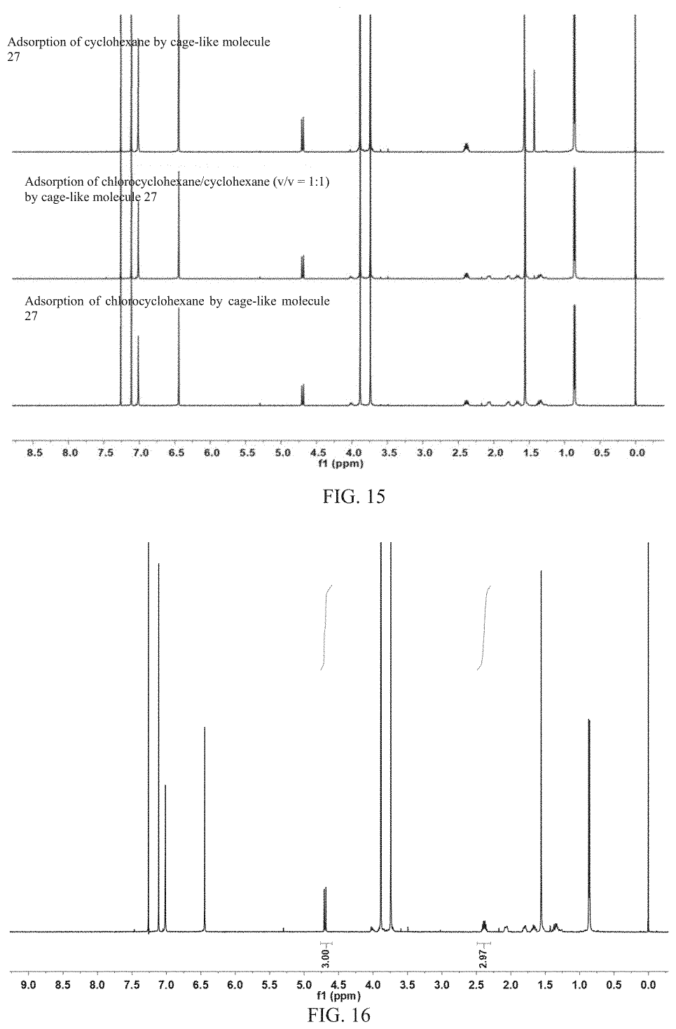

Cyclohexane is a commonly used industrial starting material and is an extremely important organic solvent. Since cyclohexene and cyclohexane have similar boiling points, cyclohexane containing a small amount of cyclohexene is usually subjected to chlorination before separation of cyclohexane from chlorocyclohexane, consuming a great deal of manpower and material resources. Therefore, the separation of chlorocyclohexane from cyclohexane is of great significance. A monocrystal of cage-like molecule 27 was activated in a vacuum oven (40° C., 2 mmHg pressure) for 10 h, and the material was placed in a vapor of cyclohexane and chlorocyclohexane (volume ratio of 1:1) for 4 h. The results show that the material can highly selectively adsorb chlorocyclohexane (the ratio of the amount of substance of chlorocyclohexane selectively adsorbed is above 95%), wherein the adsorption equivalent of the host (cage-like molecule 27) to the guest (chlorocyclohexane) is 1:1 (FIGS. 15-16).

(2) Use of Macrocyclic and Cage-Like Molecule Based on biphen[n]arene Derivative Compound

[1] Recognition of Highly Toxic Pesticide Molecules by Water-Soluble Derivative Macrocyclic Compound

1,1′-Dimethyl-4,4′-bipyridine cationic salt, also known as paraquat or purpurine, is a highly toxic pesticide. It has excellent weeding performance, but is highly irritative and corrosive to human skin and mucosa. Poisoning can be easily caused by it, and systemic poisoning will cause irreversible damage to multiple systems of the body. No effective antidote is available at present. Therefore, the development of unique water-soluble hosts for binding such water-soluble cationic compounds is of great significance, wherein host-guest studies are a scientific basis and a theoretical source for the development of antidotes and are therefore of particular importance. A series of water-soluble macrocyclic derivatives synthesized by us all have the potential to bind such molecules. Among them, macrocyclic ammonium carboxylate salt water-soluble macrocyclic compound 35 has a strong host-guest complexation effect on purpurine compounds, showing excellent prospects for application in the aspect of detoxification of paraquat pesticide. The guest has the structural formula shown below:

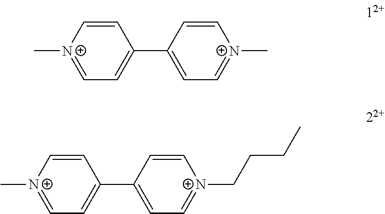

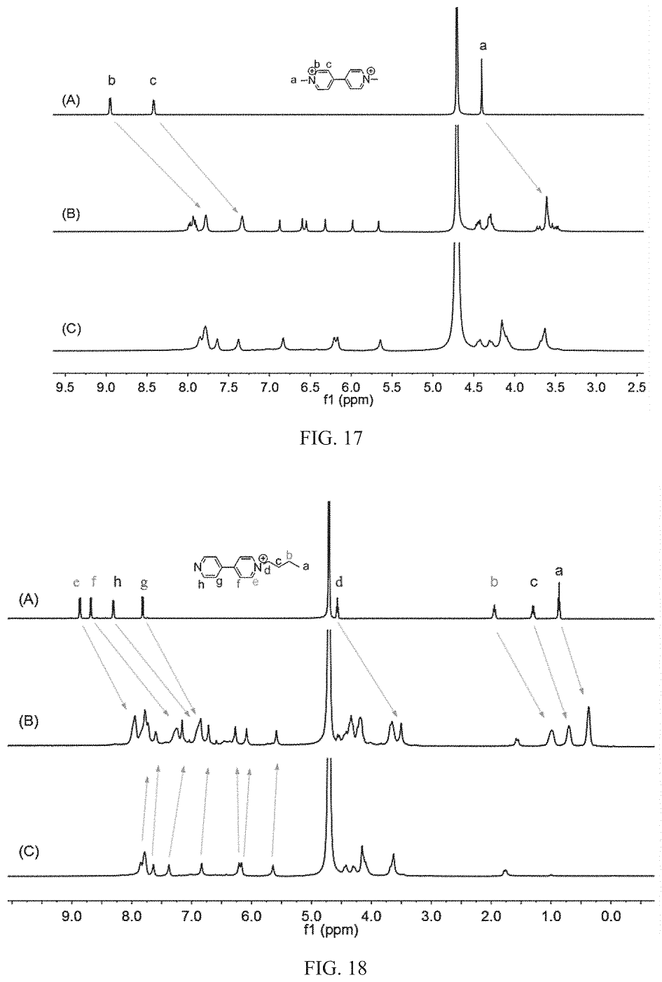

From the nuclear magnetic resonance spectra (FIGS. 17 and 18), it can be seen that there is significant binding between the host molecule and the guest molecule, their nuclear magnetic response is rapid exchange, the peak of the guest molecule shifts towards the high field, and the peak significantly broadens, indicating the formation of a host-guest clathrate. The guest completely enters the cavity of the host and thus is shielded. The signal peak part of the protons in the host molecule shifts towards the high field, indicating that π-π action is generated between the host and the guest.

[2] Recognition of Purpurine Molecule and o-Phenanthroline Cationic Derivative by Water-Soluble Cage-Like Molecule Based on Biphen[n]Arene Derivative

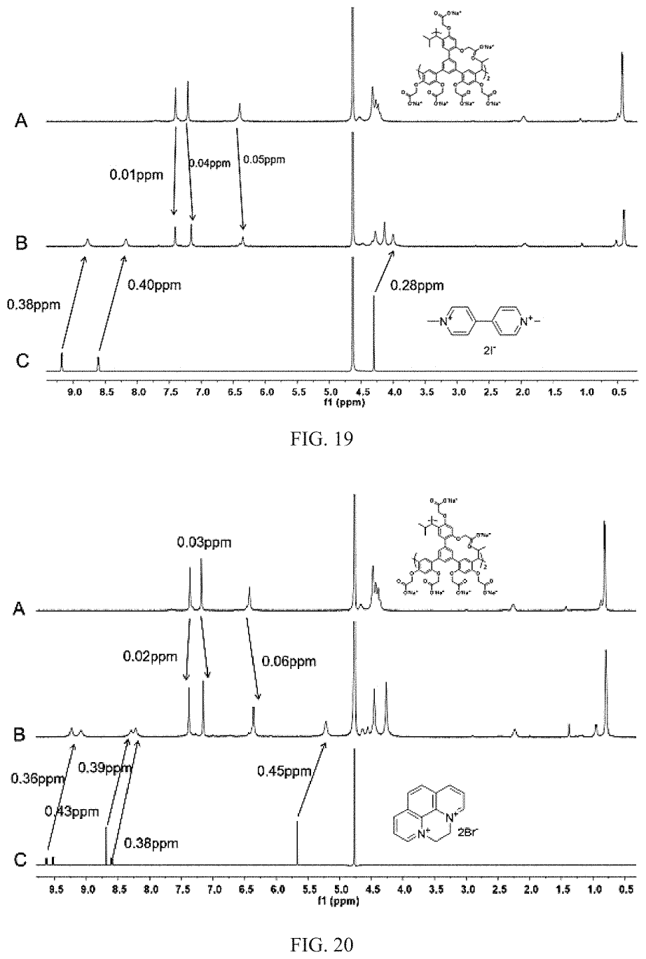

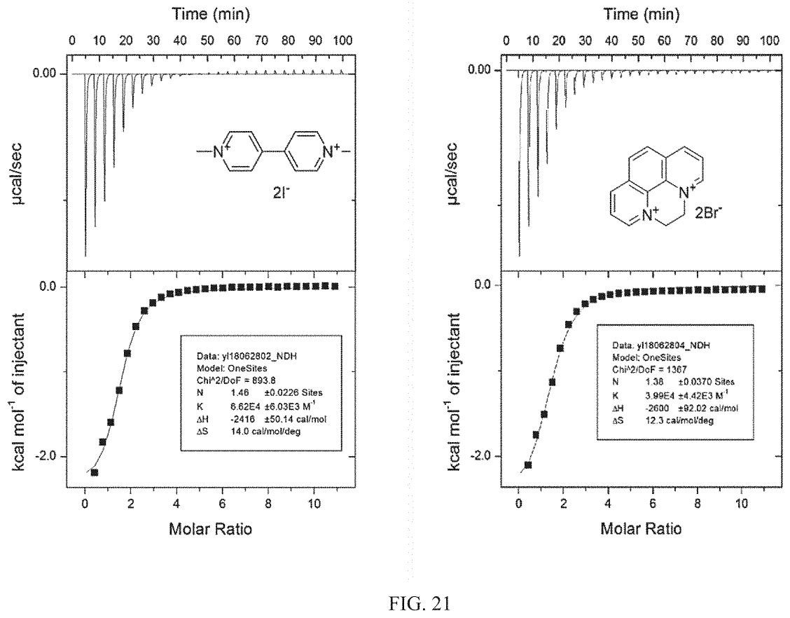

The binding capacity of macrocyclic sodium carboxylate salt water-soluble cage-like molecule 36 to paraquat molecule and o-phenanthroline cationic derivative was tested. Through nuclear magnetic resonance 1:1 experiments, it was found that water-soluble cage-like molecule compound 36 can well bind the two guest molecules (FIGS. 19 and 20). Further Isothermal Titration Calorimetry showed that the binding constant of macrocycle 36 to purpurine molecule reaches (6.62±0.60)×10⁴ M⁻¹, and the binding constant to o-phenanthroline cation reaches (3.99±0.44)×10⁴ M⁻¹ (FIG. 21).

The bind constants were calculated by isothermal titration calorimetry (ITC) measurement and fitting according to FIG. 21.

[3] Use of Special Derivative Arene in Phosphorescent Luminescent Materials

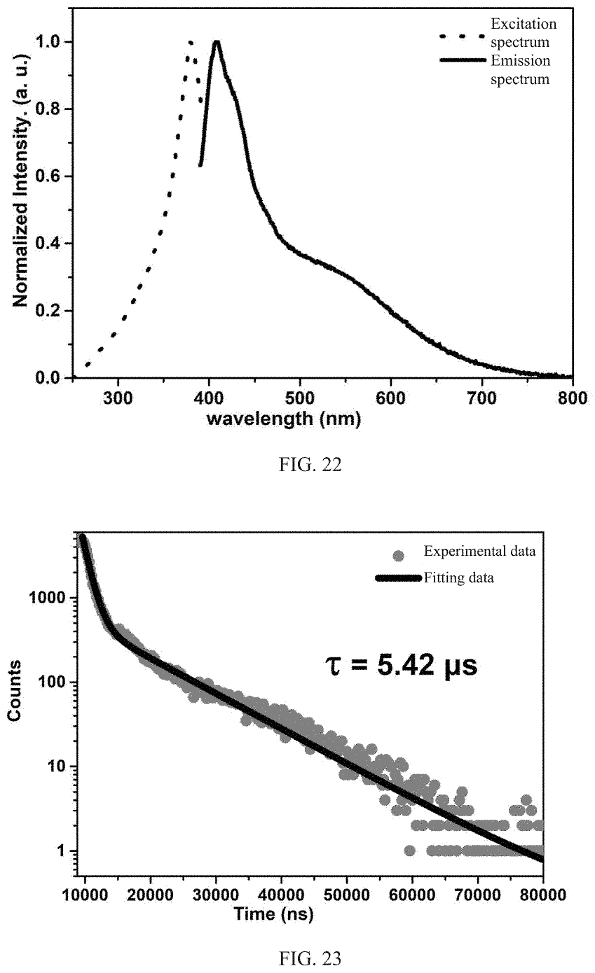

Luminescent materials are important objects and bases in the research in the field of photoelectricity. Phosphorescent materials have unique functions due to large stokes shifts and long lifetimes. It has been reported in the literature that carbazole and its derivatives exhibit phosphorescence in solid states. Therefore, we have synthesized a carbazolyl macrocyclic arene, and obtained the modified three-membered ring macrocyclic product 39 by further derivatization on the basis of the carbazolyl macrocyclic arene. The excitation spectrum shows an optimal excitation wavelength of 380 nm, and the emission spectrum shows a maximum emission wavelength of 409 nm and a shoulder peak at 505 nm (FIG. 22). The phosphorescence lifetime test shows that it has a phosphorescence lifetime in microseconds (5.42 microseconds) (FIG. 23), and is an entirely new phosphorescent material.

1. A macrocyclic and cage-like molecule based on biphen[n]arene and derivative compound thereof, having a structure as follows:
(1) a monomer of supramolecular macrocyclic and cage-like molecule based on biphen[n]arene;
(2) a supramolecular macrocyclic and cage-like molecule compound based on biphen[n]arene;
(3) a derivative compound of supramolecular macrocyclic and cage-like molecule based on biphen[n]arene; wherein,
(1) the monomer of supramolecular macrocyclic and cage-like molecule based on biphen[n]arene
[1] a macrocyclic monomer with methoxy side chain as follows: 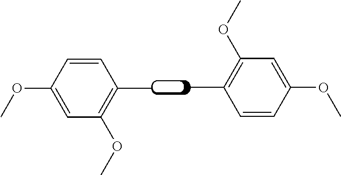
wherein 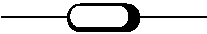
is selected from 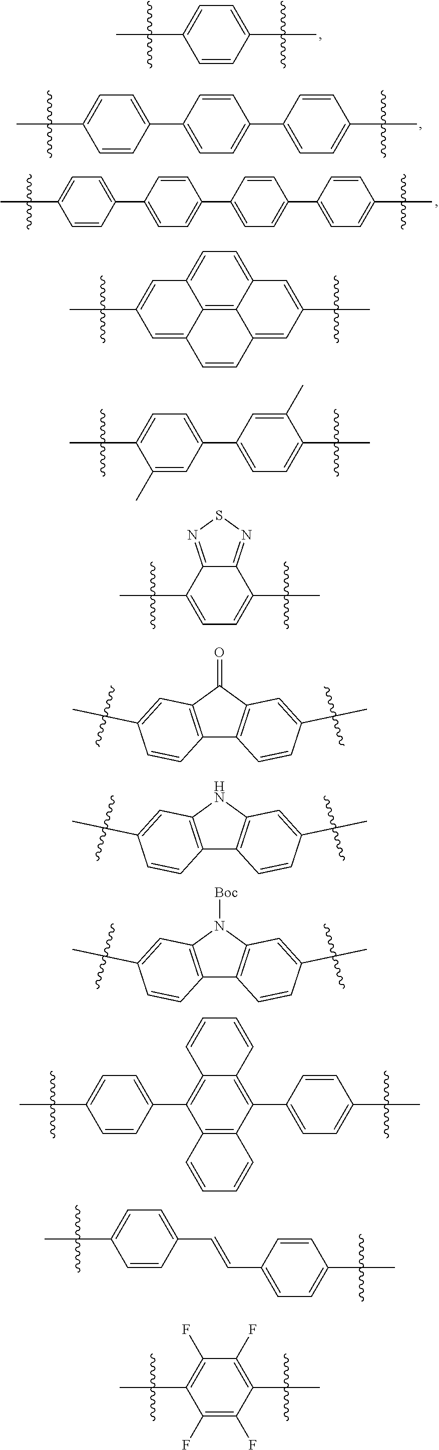 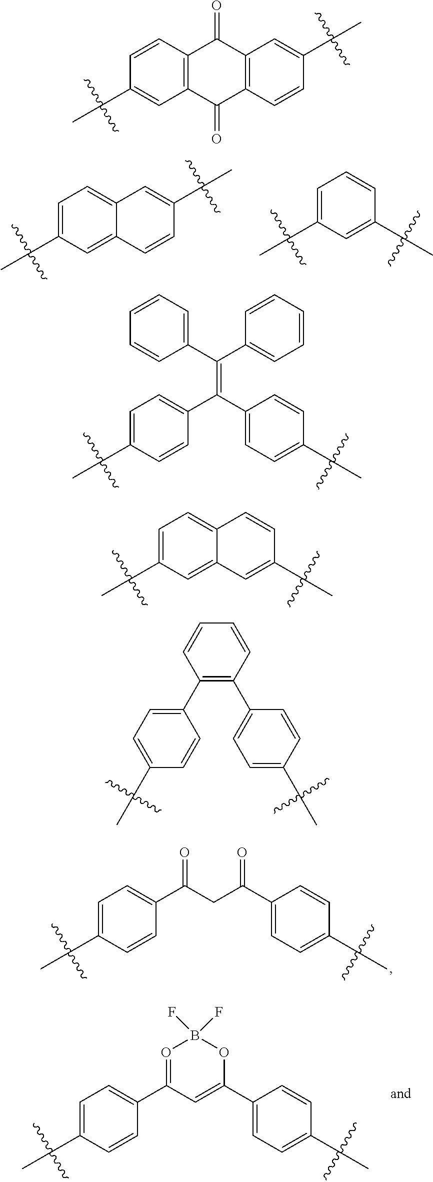 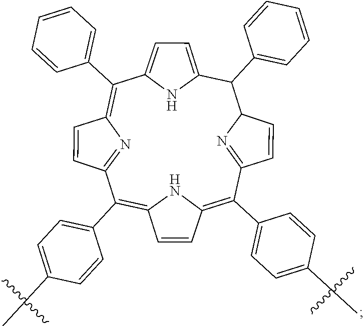 [SMILES: *c1ccc(/C2=c3\cc/c([nH]3)=C(\c3ccc(*)cc3)C3=NC(C=C3)C(c3ccccc3)c3ccc([nH]3)/C(c3ccccc3)=C3/C=CC2=N3)cc1]
[2] a monomer of cage-like molecule
wherein, 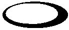
is 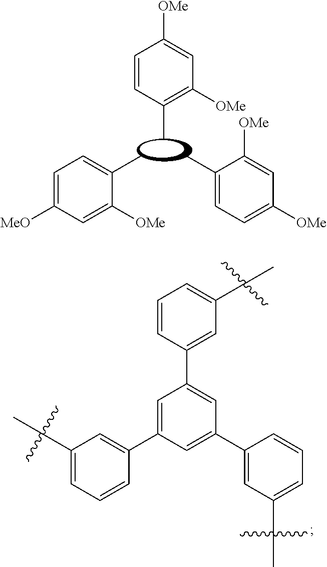
[3] a macrocyclic monomer with a dibutoxyl or 4-methoxy-2-(5-bromo-n-pentyloxy) side chain 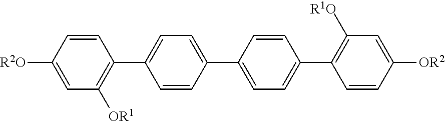
wherein R¹ and R² are selected from n-butyl; or R¹ is selected from 5-bromo-n-pentyl, and R² is selected from methyl;
(2) the supramolecular macrocyclic and cage-like molecule compound based on biphen[n]arene
[1] a macrocyclic compound with trimer or higher degree of polymerization synthesized from a linear molecule 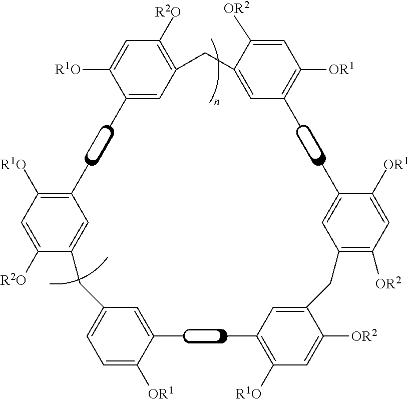 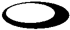
is: 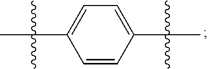 [SMILES: *c1ccc(*)cc1]
R¹=R²=Me; n=3 or 5 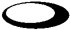
is: 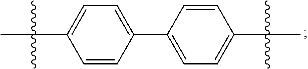 [SMILES: *c1ccc(-c2ccc(*)cc2)cc1]
R¹=R²=Me; n=3 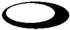
is: 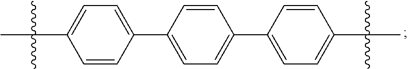 [SMILES: *c1ccc(-c2ccc(-c3ccc(*)cc3)cc2)cc1]
R¹=R²=Me; n=3-6 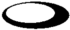
is: 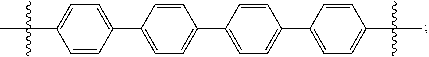 [SMILES: *c1ccc(-c2ccc(-c3ccc(-c4ccc(*)cc4)cc3)cc2)cc1]
R¹=R²=Me; n=3 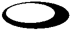
is: 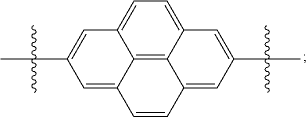 [SMILES: *c1cc2ccc3cc(*)cc4ccc(c1)c2c34]
R¹=R²=Me; n=3-4 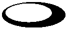
is: 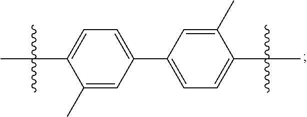 [SMILES: *c1ccc(-c2ccc(*)c(C)c2)cc1C]
R¹=R²=Me; n=3-6 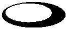
is: 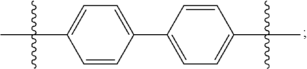 [SMILES: *c1ccc(-c2ccc(*)cc2)cc1]
R¹=R²=n-Butyl; n=3 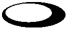
is: 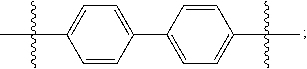 [SMILES: *c1ccc(-c2ccc(*)cc2)cc1]
R¹=5-bromo-n-pentyl; R²=Me; n=3-4 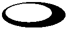
is: 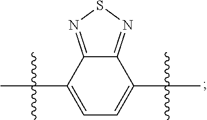 [SMILES: *c1ccc(*)c2nsnc12]
R¹=R²=Me; n=3 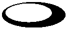
is: 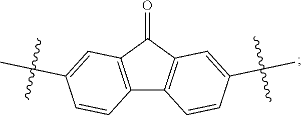 [SMILES: *c1ccc2c(c1)C(=O)c1cc(*)ccc1-2]
R¹=R²=Me; n=2-5 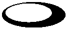
is: 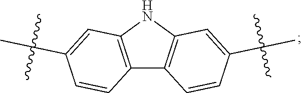 [SMILES: *c1ccc2c(c1)[nH]c1cc(*)ccc12]
R¹=R²=Me; n=3 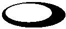
is: 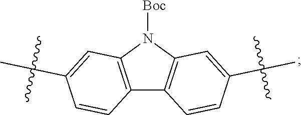 [SMILES: *c1ccc2c3ccc(*)cc3n(C(=O)OC(C)(C)C)c2c1]
R¹=R²=Me; n=3 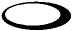
is: 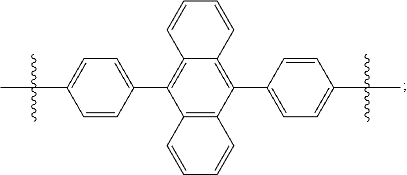 [SMILES: *c1ccc(-c2c3ccccc3c(-c3ccc(*)cc3)c3ccccc23)cc1]
R¹=R²=Me; n=3 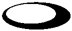
is: 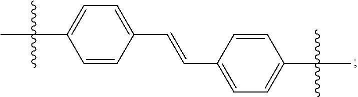 [SMILES: *c1ccc(/C=C/c2ccc(*)cc2)cc1]
R¹=R²=Me; n=3 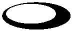
is: 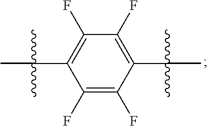 [SMILES: *c1c(F)c(F)c(*)c(F)c1F]
R¹=R²=Me; n=3 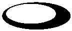
is: 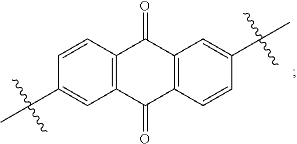 [SMILES: *c1ccc2c(c1)C(=O)c1ccc(*)cc1C2=O]
R¹=R²=Me; n=3 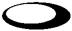
is: 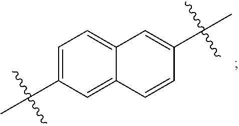 [SMILES: *c1ccc2cc(*)ccc2c1]
R¹=R²=Me; n=3
and 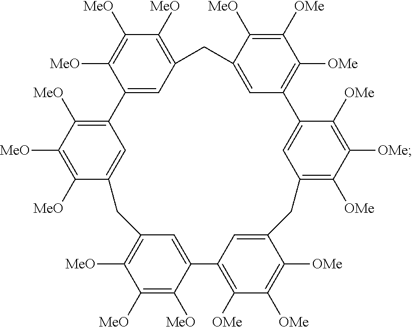 [SMILES: COc1c2cc(c(OC)c1OC)-c1cc(c(OC)c(OC)c1OC)Cc1cc(c(OC)c(OC)c1OC)-c1cc(c(OC)c(OC)c1OC)Cc1cc(c(OC)c(OC)c1OC)-c1cc(c(OC)c(OC)c1OC)C2]
[2] a dimeric supramolecular macrocyclic compound prepared from a V-shaped molecule 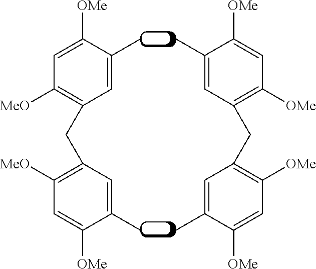
specific structures are as follows: 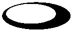
is: 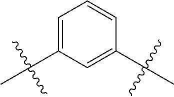 [SMILES: *c1cccc(*)c1] 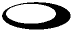
is: 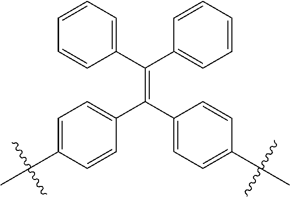 [SMILES: *c1ccc(C(=C(c2ccccc2)c2ccccc2)c2ccc(*)cc2)cc1]
is: 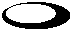
is: 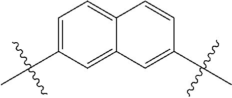 [SMILES: *c1ccc2ccc(*)cc2c1] 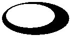
is: 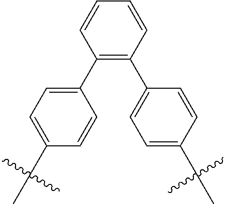 [SMILES: *c1ccc(-c2ccccc2-c2ccc(*)cc2)cc1] 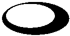
is: 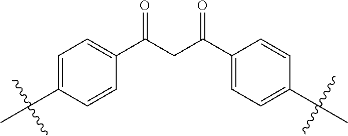 [SMILES: *c1ccc(C(=O)CC(=O)c2ccc(*)cc2)cc1] 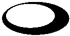
is: 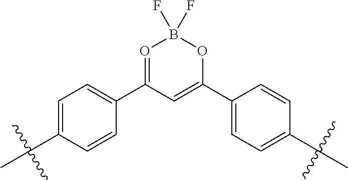 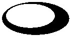
is: 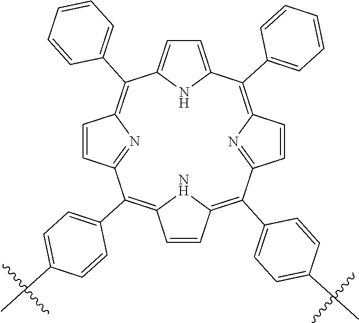 [SMILES: *c1ccc(-c2c3nc(c(-c4ccccc4)c4ccc([nH]4)c(-c4ccccc4)c4nc(c(-c5ccc(*)cc5)c5ccc2[nH]5)C=C4)C=C3)cc1]
[3] a supracage-like molecule compound constructed from monomer molecules having three 2,4-dialkoxyphenyl groups: 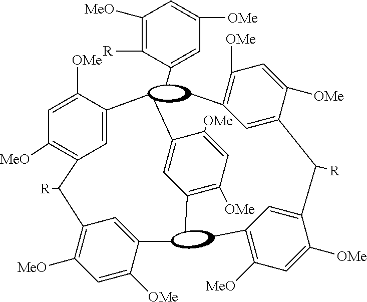
specific structures are as follows: 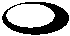
is: 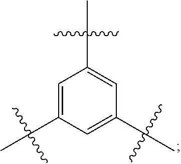 [SMILES: *c1cc(*)cc(*)c1]
R=H 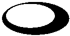
is: 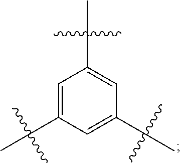 [SMILES: *c1cc(*)cc(*)c1]
R= 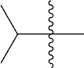 [SMILES: *C(C)C] 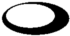
is: 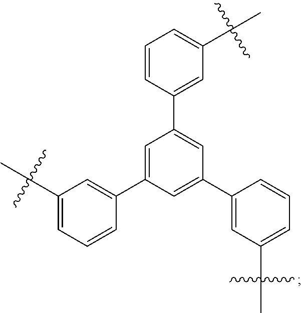 [SMILES: *c1cccc(-c2cc(-c3cccc(*)c3)cc(-c3cccc(*)c3)c2)c1]
R=H;
[4] a supramolecular macrocyclic compound having different repeat units, obtained by regulating proportions of different monomer molecules to achieve copolymerization of the different monomers: 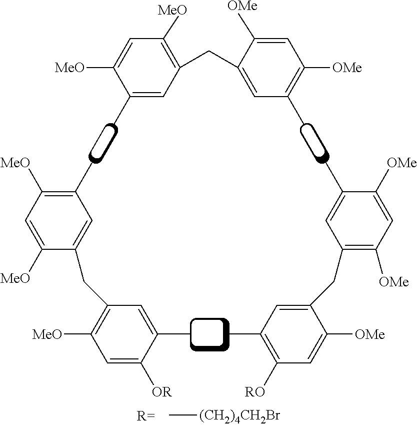
wherein 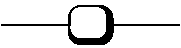
is selected from 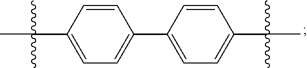 [SMILES: *c1ccc(-c2ccc(*)cc2)cc1] 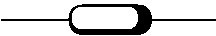
is selected from 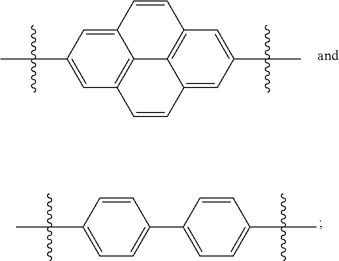 [SMILES: *c1cc2ccc3cc(*)cc4ccc(c1)c2c34 ; *c1ccc(-c2ccc(*)cc2)cc1]
(3) the derivative compound of supramolecular macrocyclic and cage-like molecule based on biphen[n]arene
[1] synthesis of a hydroxyl macrocyclic compound
1) synthesis of a trimeric macrocyclic hydroxyl compound formed from linear monomers: 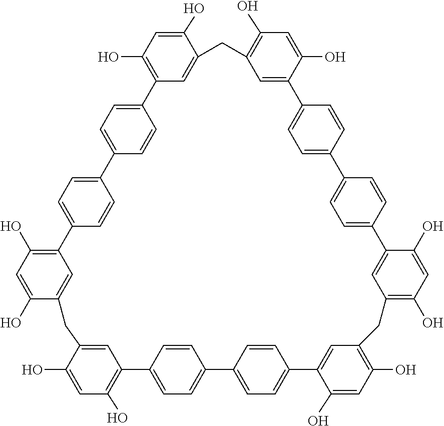 [SMILES: Oc1cc(O)c2cc1Cc1cc(c(O)cc1O)-c1ccc(cc1)-c1ccc(cc1)-c1cc(c(O)cc1O)Cc1cc(c(O)cc1O)-c1ccc(cc1)-c1ccc(cc1)-c1cc(c(O)cc1O)Cc1cc(c(O)cc1O)-c1ccc(cc1)-c1ccc-2cc1]
2) a V-shaped dimeric macrocyclic hydroxyl compound 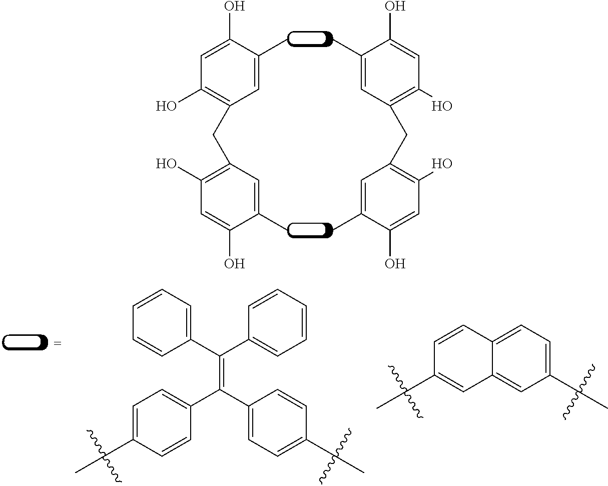
3) a cage-like molecule hydroxyl derivative compound: 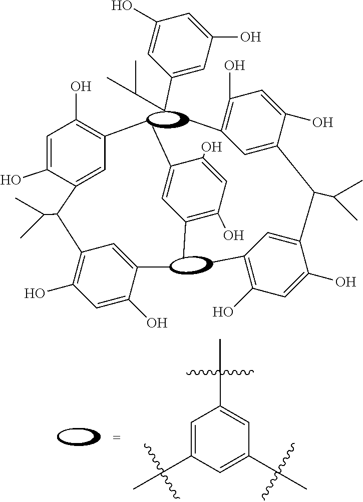
[2] a water-soluble macrocyclic and cage-like molecule compound
1) a water-soluble ammonium carboxylate derivative macrocyclic compound: 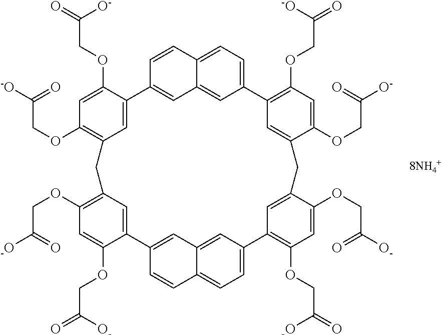
2) a water-soluble sodium carboxylate derivative cage-like molecule compound: 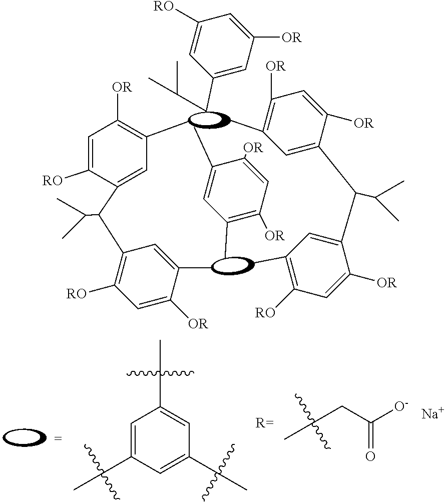
3) a water-soluble sulfonate salt derivative macrocyclic compound 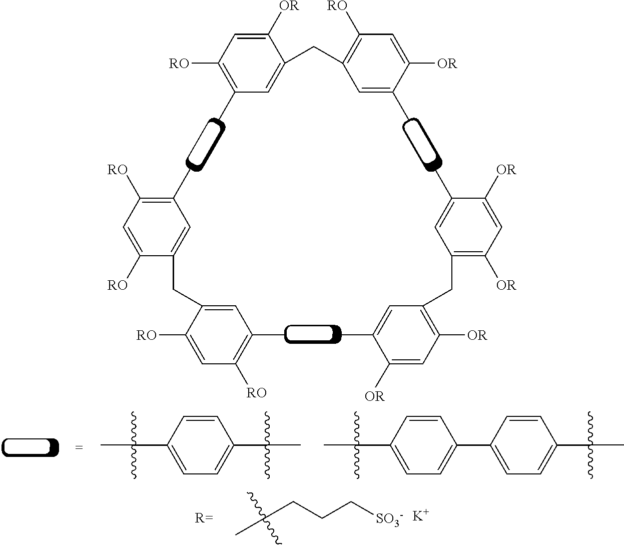
[3] special derivatization of some macrocyclic compounds:
1) a carbazole derivative macrocyclic compound 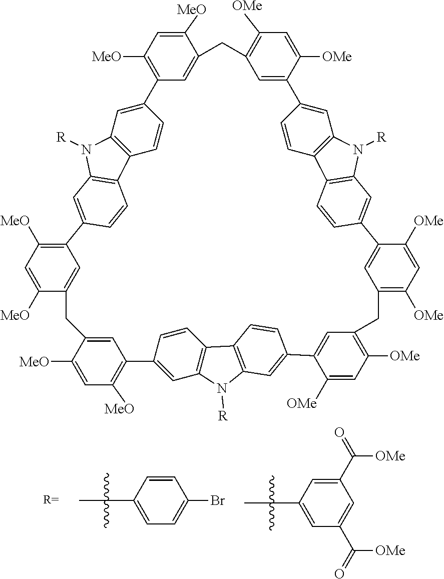
2) a pyridine derivative macrocyclic compound:

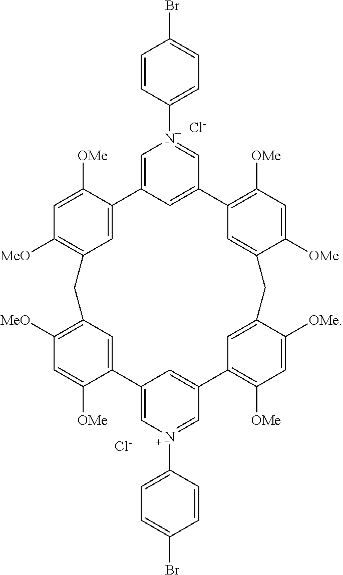

2. A synthesis method for the macrocyclic and cage-like molecule based on biphen[n]arene and derivative compound thereof according to claim 1, wherein a bis-(2,4-dialkoxyphenyl)arene or tris-(2,4-dialkoxyphenyl)arene is dissolved in a halohydrocarbon solvent, an aldehyde reactant is added, a series of macrocycle hosts and cage-like molecule compounds based on biphen[n]arene are obtained through cyclization under catalysis by a Lewis acid, and a macrocyclic and cage-like compound based on biphen[n]arene is obtained, which can be further derivatized to obtain a derivative compound of the macrocyclic and cage-like compound; the halohydrocarbon solvent is at least one selected from: dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, and monobromodecane; the aldehyde reactant can be selected from paraformaldehyde and isobutyraldehyde.

3. The synthesis method for the macrocyclic and cage-like molecule based on biphen[n]arene and derivative compound thereof according to claim 2, comprising the following aspects:
(1) a synthesis method of the monomer of supramolecular macrocyclic and cage-like molecule based on biphen[n]arene;
(2) a synthesis method of the supramolecular macrocyclic and cage-like molecule based on biphen[n]arene; and
(3) a synthesis method of the derivatization of macrocyclic and cage-like molecule based on biphen[n]arenes;
wherein the synthesis method of the monomer of supramolecular macrocyclic and cage-like molecule based on biphen[n]arene of step (1) is as follows:
[1] preparation of a monomer of supramolecular macrocycle based on biphen[n]arene
dissolving a dibromide or an iodide and 2,4-dimethoxyphenylboronic acid in an aqueous solution of dioxane (dioxane:water=5:1), then adding tetrakis(triphenylphosphine)palladium catalyst and sodium carbonate, and stirring the mixture at reflux overnight; after the reaction is completed, cooling the reaction mixture to room temperature, removing the solvent by rotary evaporation, dissolving the residue in dichloromethane, and washing the resulting solution with water three times; drying the organic layer over anhydrous Na₂SO₄ and removing the solvent again by rotary evaporation, and preparing the residue for column chromatography to isolate the monomer;
[2] preparation of a monomer of the cage-like molecule
dissolving a tribromide and 2,4-dimethoxyphenylboronic acid in an aqueous solution of dioxane (dioxane water=5:1), then adding tetrakis(triphenylphosphine)palladium catalyst and sodium carbonate, and stirring the mixture at reflux overnight; after the reaction is completed, cooling the reaction mixture to room temperature, removing the solvent by rotary evaporation, dissolving the residue in dichloromethane, and washing the resulting solution with water three times; drying the organic layer over anhydrous Na₂SO₄ and removing the solvent again by rotary evaporation, and preparing the residue for column chromatography to isolate the monomer;
[3] preparation of a macrocyclic monomer with dibutoxyl or 4-methoxy-2-(5-bromo-n-pentyloxy) side chain
(1) synthesis of a monomer with dibutoxy side chain
adding excessively n-butyl bromide to a three-necked flask and heating n-butyl bromide at reflux, starting dissolving 4-bromo-resorcinol in acetonitrile and adding dropwise the resulting solution to the reaction system, and allowing the reaction system to react overnight; after the reaction is completed, stopping the heating, and filtering the mixture to remove potassium carbonate; concentrating the reaction solution by rotary evaporation, and subjecting the residue to column chromatography to isolate 4-bromo-1,3-dibutoxybenzene reaction product; subsequently, dissolving completely 4-bromo-1,3-dibutoxybenzene in an aqueous solution of 1,4-dioxane (dioxane:water=5:1), then adding 4,4′-biphenyldiboronic acid, tetrakis(triphenylphosphine)palladium and sodium carbonate, and heating the mixed system to 100° C. and refluxing overnight; after the reaction is completed, cooling the reaction mixture to room temperature, removing the solvent by rotary evaporation, dissolving the residue in dichloromethane, and washing the resulting solution with water three times; drying the organic layer over anhydrous Na₂SO₄ and removing the solvent again by rotary evaporation, and preparing the residue for column chromatography to isolate the monomer;
(2) synthesis of a monomer with 4-methoxy-2-(5-bromo-n-pentyloxy) side chain
adding excessively 1,5-dibromopentane to a three-necked flask and heating 1,5-dibromopentane at reflux, starting dissolving 2-bromo-5-methoxyphenol in acetonitrile and adding dropwise the resulting solution to the reaction system; allowing the reaction system to react overnight; after the reaction is completed, stopping the heating, and filtering the mixture to remove potassium carbonate; concentrating the reaction solution by rotary evaporation, and subjecting the residue to column chromatography to isolate 4-methoxy-2-(5-bromo-n-pentyloxy)bromobenzene reaction product; subsequently, dissolving completely 4-methoxy-2-(5-bromo-n-pentyloxy)bromobenzene in an aqueous solution of 1,4-dioxane (dioxane:water=5:1), then adding 2,4-dimethoxyphenylboronic acid, tetrakis(triphenylphosphine)palladium and sodium carbonate, and heating the mixed system to 100° C. and refluxing overnight; after the reaction is completed, cooling the reaction mixture to room temperature, removing the solvent by rotary evaporation, dissolving the residue in dichloromethane, and washing the resulting solution with water three times; drying the organic layer over anhydrous Na₂SO₄ and removing the solvent again by rotary evaporation, and preparing the residue for column chromatography to isolate the monomer;
wherein the synthesis method of the supramolecular macrocyclic and cage-like molecule based on biphen[n]arene of step (2) is as follows:
[1] synthesis of a supramolecular macrocycle with trimer or higher degree of polymerization from a molecule having a linear structure:
dissolving a bis-(2,4-dialkoxyphenyl)arene having a linear structure and paraformaldehyde in a haloalkane solvent, adding a Lewis acid catalyst after dissolution, monitoring the reaction by thin-layer chromatography (TLC), quenching the reaction with saturated aqueous sodium bicarbonate solution after the reaction is completed, washing the reaction mixture with saturated aqueous sodium chloride solution, drying the reaction mixture over anhydrous sodium sulfate, and subjecting the resulting mixture to silica gel chromatography to isolate a ring formation product with trimer or higher degree of polymerization; wherein the halohydrocarbon solvent is at least one selected from: dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, and monobromodecane; the aldehyde reactant is selected from paraformaldehyde and isobutyraldehyde;
dissolving 2,2′-, 3,3′- or 4,4′-hexamethoxybiphenyl and paraformaldehyde in a haloalkane solvent, adding a Lewis acid catalyst after dissolution, monitoring the reaction by thin-layer chromatography (TLC), quenching the reaction with saturated aqueous sodium bicarbonate solution after the reaction is completed, washing the reaction mixture with saturated aqueous sodium chloride solution, drying the reaction mixture over anhydrous sodium sulfate, and subjecting the resulting mixture to silica gel chromatography to isolate a ring formation product with trimer or higher degree of polymerization; wherein the halohydrocarbon solvent is at least one selected from: dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, and monobromodecane; the aldehyde reactant is selected from paraformaldehyde and isobutyraldehyde;
[2] preparation of a dimeric macrocyclic arene from a V-shaped monomer:
dissolving a bis-(2,4-dialkoxyphenyl)arene having a V-shaped structure and paraformaldehyde in a haloalkane solvent, adding a Lewis acid catalyst after dissolution, monitoring the reaction by thin-layer chromatography (TLC), quenching the reaction with saturated aqueous sodium bicarbonate solution after the reaction is completed, washing the reaction mixture with saturated aqueous sodium chloride solution, drying the reaction mixture over anhydrous sodium sulfate, and subjecting the resulting mixture to silica gel chromatography to isolate a dimeric ring formation product, wherein the halohydrocarbon solvent is at least one selected from: dichloromethane, dibromomethane, trichloromethane, tribromoalkane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, and monobromodecane; the aldehyde reactant is selected from paraformaldehyde and isobutyraldehyde;
[3] synthesis of a cage-like macrocyclic arene from tris-(2,4-dialkoxyphenyl)arene:
dissolving a tris-(2,4-dialkoxyphenyl)arene and paraformaldehyde or isobutyraldehyde in a haloalkane solvent in a molar ratio of about 1:5, adding a Lewis acid catalyst after dissolution, monitoring the reaction by thin-layer chromatography (TLC), quenching the reaction with saturated aqueous sodium bicarbonate solution after the reaction is completed, washing the reaction mixture with saturated aqueous sodium chloride solution, drying the reaction mixture over anhydrous sodium sulfate, and subjecting the resulting mixture to silica gel chromatography to isolate a cage-like molecule compound product; wherein the halohydrocarbon solvent is at least one selected from: dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, and monobromodecane; the aldehyde reactant is selected from paraformaldehyde and isobutyraldehyde;
[4] obtaining of a supramolecular macrocyclic compound in which a macrocycle has different units by regulating proportions of different monomer molecules to achieve copolymerization of the different monomers: adding two bis-(2,4-dialkoxyphenyl)arenes to a reaction flask in a molar ratio of 1:5, then adding paraformaldehyde with an equivalent that is twice the total amount of substance of the two derivatives, adding a Lewis acid catalyst after dissolution in a haloalkane, monitoring the reaction by thin-layer chromatography (TLC), quenching the reaction with saturated aqueous sodium bicarbonate solution after the reaction is completed, washing the reaction mixture with saturated aqueous sodium chloride solution, drying the reaction mixture over anhydrous sodium sulfate, and subjecting the resulting mixture to silica gel chromatography to isolate a copolymeric three-membered macrocyclic compound; wherein the halohydrocarbon solvent is at least one selected from: dichloromethane, dibromomethane, trichloromethane, tribromomethane, tetrachloromethane, dichloroethane, dibromoethane, trichloroethane, tribromoethane, tetrachloroethane, tetrabromoethane, monochloropropane, monobromopropane, monochlorobutane, monobromobutane, monochloropentane, monobromopentane, monochlorohexane, monobromohexane, monochloroheptane, monobromoheptane, monochlorooctane, monobromooctane, monochlorononane, monobromononane, monochlorodecane, and monobromodecane;
wherein the synthesis method for the derivatization of macrocyclic and cage-like molecule based on biphen[n]arenes of step (3) is as follows:
[1] synthesis of a hydroxyl macrocyclic compound:
dissolving a biphenyl arene macrocycle in dichloromethane, adding 20 equivalents of boron tribromide compound to the reaction system, after 1 day of reaction, adding dropwise the reaction mixture to a mixture of ice and water to precipitate a light purple powder, and performing suction filtration to obtain a hydroxyl biphenyl arene macrocycle product:
[2] synthesis of a carboxylic acid water-soluble macrocycle and a carboxylic acid water-soluble cage-like molecule:
dissolving the hydroxyl macrocyclic compound product in acetonitrile or acetone, then adding K₂CO₃, refluxing the mixture for 2 h, adding ethyl bromoacetate, refluxing the mixture for another 48 h, cooling the reaction mixture to room temperature after the reaction is completed, filtering the reaction mixture, washing with dichloromethane multiple times, removing the solvent by vacuum rotary evaporation, adding a small amount of dichloromethane so that the solid is just dissolved, then adding a large amount of petroleum ether so that a large amount of solid is subsequently precipitated, and performing suction filtration under reduced pressure to obtain the desired product; dissolving the product in a mixed solution of 50 mL of THF and 20 mL of an aqueous solution of sodium hydroxide (a mass concentration of 20%), stirring the resulting solution at reflux for 10 h, removing THF by rotary evaporation, adding 20 mL of water, adding hydrochloric acid for acidification until a pH test paper shows weak acidity, performing suction filtration under reduced pressure to obtain the desired product, and then adding gradually the carboxylic acid derivative macrocyclic and cage-like molecule to an alkali solution to obtain a carboxylate salt water-soluble macrocyclic and cage-like molecule compound of the corresponding alkali;
[3] synthesis of a sulfonated water-soluble macrocycle
dissolving a hydroxyl macrocyclic compound in acetone, adding K₂CO₃, stirring the mixture at reflux for 2 h, then adding 1 equivalent of propane sultone, stirring the mixture at reflux for another 3 days, cooling the mixture to room temperature after the reaction is completed, performing suction filtration, washing the filter cake twice with acetone, dissolving the resulting filter cake in water, purifying the resulting solution to remove potassium carbonate by about one week of dialysis with a dialysis bag; adding 800 mL of distilled water to a 1 L large beaker, then placing the dialysis bag in the water, fixing slightly the dialysis bag with a rubber band, adding a stirrer, stirring continuously the water, replacing the water in the beaker once every 2 hours, reducing the water replacing frequency after one day to once every half a day, and replacing the water once on the third day; and finally concentrating the aqueous solution in the dialysis bag by rotary evaporation to obtain a sulfonated water-soluble macrocycle product;
[4] special derivatization of certain macrocyclic compounds
[1] synthesis of a carbazole derivative macrocycle:
1) monomer modification, and then ring closing:
dissolving bis-(2,4-dialkoxyphenyl)carbazole and 2 equivalents of p-dibromobenzene or methyl 5-bromoisophthalate in N,N-dimethylacetamide, adding 1 equivalent of copper(I) iodide and 6 equivalents of potassium carbonate, heating the mixture to 180° C. under nitrogen (argon) atmosphere for 24 h of reaction, cooling the mixture to room temperature after the reaction is completed, pouring the product into a saturated aqueous solution of NaCl, performing extraction with dichloromethane three times, performing drying over anhydrous sodium sulfate, and performing purification by column chromatography to obtain a product in the form of a white solid; dissolving the monomer above and 3 equivalents of an aldehyde compound in a haloalkane, adding a Lewis acid catalyst, monitoring the reaction by thin-layer chromatography (TLC); after the reaction is completed, quenching the reaction with saturated aqueous sodium bicarbonate solution, washing the mixture with saturated aqueous sodium chloride solution, drying the mixture over anhydrous sodium sulfate, and subjecting the resulting mixture to silica gel chromatography to isolate a carbazole three-membered ring macrocycle product modified by p-dibromobenzene or methyl 5-bromoisophthalate;
2) ring closing, and then modification:
dissolving bis-(2,4-dialkoxyphenyl)carbazole and 3 equivalents of an aldehyde compound in a haloalkane, adding 2 equivalents of a Lewis acid catalyst, monitoring the reaction by thin-layer chromatography (TLC), quenching the reaction with saturated aqueous sodium bicarbonate solution after the reaction is completed, washing the mixture with saturated aqueous sodium chloride solution, drying the mixture over anhydrous sodium sulfate, and subjecting the resulting mixture to silica gel chromatography to isolate a carbazole-modified three-membered ring product; dissolving a mixture of the carbazole-modified three-membered ring, 2 equivalents of p-dibromobenzene or methyl 5-bromoisophthalate, 1 equivalent of copper(I) iodide and 6 equivalents of potassium carbonate in N,N-dimethylacetamide, heating the resulting solution to 180° C. under nitrogen (argon) atmosphere for 24 h of reaction, then cooling the reaction solution to room temperature, pouring the reaction solution into a saturated aqueous solution of NaCl, performing extraction with dichloromethane three times, performing drying over anhydrous sodium sulfate, and performing purification by column chromatography to obtain a three-membered ring product modified by p-dibromobenzene or methyl 5-bromoisophthalate;
[2] synthesis of a pyridine derivative macrocycle:
1) monomer modification and then ring closing:
adding sequentially bis-(2,4-dimethoxyphenyl)pyridine monomer and 2,4-dinitrochlorobenzene to a 50 mL round-bottomed flask, adding 5 mL of acetone, mixing well the mixture by ultrasonication, then heating the reactants at reflux overnight, after the reaction is completed, removing the solvent by rotary evaporation, adding a large amount of ethyl acetate, performing suction filtration, adding the filter cake to an acetonitrile solution, performing suction filtration, collecting the filtrate, concentrating the filtrate by rotary evaporation, adding a small amount of methanol to completely dissolve the residue, adding ethyl acetate, stirring the mixture for 1-4 h, and performing suction filtration to obtain an intermediate product; adding the intermediate product to a 50 mL round-bottomed flask, adding 1 mL of ethanol, then adding 3 mL of water, mixing well the mixture, then adding p-bromophenylamine, heating the mixture at reflux under nitrogen atmosphere for 1-2 days, cooling the mixture to room temperature, then adding ethyl acetate, and performing suction filtration; adding ethanol to the filtrate, removing the solvent by rotary evaporation, adding a small amount of acetone to dissolve the solid, then adding a large amount of ethyl acetate, and performing suction filtration to obtain a modified pyridine arene derivative monomer; then weighing 1 g of the derivative arene monomer and paraformaldehyde, pouring a haloalkane as a solvent for dissolution, adding 1.5 equivalents of a Lewis acid catalyst while stirring, monitoring the reaction by thin-layer chromatography, after 30 min of reaction, adding a saturated solution of sodium bicarbonate to quench the reaction, washing the mixture with 50 mL of a saturated solution of sodium chloride, drying the mixture over anhydrous sodium sulfate, and subjecting the resulting mixture to silica gel chromatography to obtain the target product;
2) ring closing, and then modification:
adding sequentially a pyridine derivative macrocycle and 2,4-dinitrochlorobenzene to a 50 mL round-bottomed flask, adding 5 mL of acetone, mixing well the mixture by ultrasonication, and then heating the reactants at reflux overnight; after the reaction is completed, removing the solvent by rotary evaporation, adding a large amount of ethyl acetate, performing suction filtration, dissolving the filter cake in acetonitrile, performing suction filtration, drying the filtrate by rotary evaporation, dissolving the residue in a small amount of methanol, adding ethyl acetate, stirring the mixture for 1-4 h, and performing suction filtration to obtain an intermediate product; adding the intermediate product to 1 mL of ethanol, then adding 3 mL of water, mixing well the mixture, then adding p-bromophenylamine, and refluxing the mixture under nitrogen atmosphere for 1-2 days; after the reaction is completed, cooling the mixture to room temperature, adding ethyl acetate, performing suction filtration, and removing the solvent by rotary evaporation; adding a small amount of acetone to dissolve the solid, then adding a large amount of ethyl acetate to precipitate a solid, and performing suction filtration to obtain the target product.

4. Use of the macrocyclic and cage-like molecule based on biphen[n]arene and derivative compound thereof according to claim 1 in materials, environment and biology.

5. The use according to claim 4, wherein the macrocyclic compound based on biphen[n]arene is used as an adsorptive separation material for trimethylbenzene isomer or for the recognition of an ammonium cationic compound.

6. The use according to claim 4, wherein the macrocyclic compound based on biphen[n]arene is used for the adsorptive separation of cyclohexane and chlorocyclohexane.

7. The use according to claim 4, wherein the macrocyclic and cage-like molecule based on biphen[n]arene and derivative compound thereof is used for the recognition of a toxic cationic derivative such as purpurine molecule and o-phenanthroline.

8. The use according to claim 4, wherein the derived macrocyclic arene is used as a phosphorescent luminescent material.
